SEQUENCE LISTING (9 sequences)

SEQ ID NO: 1 - protein, 352 aa, Homo sapiens
MEGISIYTSDNYTEEMGSGDYDSMKEPCFREENANFNKIFLPTIYSIIFLTGIVGNGLVILVMGYQKKLRSMTDKYRLHLSVADLLFVITLPFWAVDAVANWYFGNFLCKAVHVIYTVNLYSSVLILAFISLDRYLAIVHATNSQRPRKLLAEKVVYVGVWIPALLLTIPDFIFANVSEADDRYICDRFYPNDLWVVVFQFQHIMVGLILPGIVILSCYCIIISKLSHSKGHQKRKALKTTVILILAFFACWLPYYIGISIDSFILLEIIKQGCEFENTVHKWISITEALAFFHCCLNPILYAFLGAKFKTSAQHALTSVSRGSSLKILSKGKRGGHSSVSTESESSSFHSS

SEQ ID NO: 2 - DNA, 1691 bp, Homo sapiens
aacttcagtttgttggctgcggcagcaggtagcaaagtgacgccgagggcctgagtgctccagtagccaccgcatctggagaaccagcggttaccatggaggggatcagtatatacacttcagataactacaccgaggaaatgggctcaggggactatgactccatgaaggaaccctgtttccgtgaagaaaatgctaatttcaataaaatcttcctgcccaccatctactccatcatcttcttaactggcattgtgggcaatggattggtcatcctggtcatgggttaccagaagaaactgagaagcatgacggacaagtacaggctgcacctgtcagtggccgacctcctctttgtcatcacgcttcccttctgggcagttgatgccgtggcaaactggtactttgggaacttcctatgcaaggcagtccatgtcatctacacagtcaacctctacagcagtgtcctcatcctggccttcatcagtctggaccgctacctggccatcgtccacgccaccaacagtcagaggccaaggaagctgttggctgaaaaggtggtctatgttggcgtctggatccctgccctcctgctgactattcccgacttcatctttgccaacgtcagtgaggcagatgacagatatatctgtgaccgcttctaccccaatgacttgtgggtggttgtgttccagtttcagcacatcatggttggccttatcctgcctggtattgtcatcctgtcctgctattgcattatcatctccaagctgtcacactccaagggccaccagaagcgcaaggccctcaagaccacagtcatcctcatcctggctttcttcgcctgttggctgccttactacattgggatcagcatcgactccttcatcctcctggaaatcatcaagcaagggtgtgagtttgagaacactgtgcacaagtggatttccatcaccgaggccctagctttcttccactgttgtctgaaccccatcctctatgctttccttggagccaaatttaaaacctctgcccagcacgcactcacctctgtgagcagagggtccagcctcaagatcctctccaaaggaaagcgaggtggacattcatctgtttccactgagtctgagtcttcaagttttcactccagctaacacagatgtaaaagacttttttttatacgataaataacttttttttaagttacacatttttcagatataaaagactgaccaatattgtacagtttttattgcttgttggatttttgtcttgtgtttctttagtttttgtgaagtttaattgacttatttatataaattttttttgtttcatattgatgtgtgtctaggcaggacctgtggccaagttcttagttgctgtatgtctcgtggtaggactgtagaaaagggaactgaacattccagagcgtgtagtgaatcacgtaaagctagaaatgatccccagctgtttatgcatagataatctctccattcccgtggaacgtttttcctgttcttaagacgtgattttgctgtagaagatggcacttataaccaaagcccaaagtggtatagaaatgctggtttttcagttttcaggagtgggttgatttcagcacctacagtgtacagtcttgtattaagttgttaataaaagtacatgttaaacttaaaaaaaaaaaaaaaaaa

SEQ ID NO: 3 - protein, 922 aa, Homo sapiens
MQNIMNDMPIYMYSVCNVMSGDQDNWLRTNWVYRGEAERIFIELKFTVRDCNSFPGGASSCKETFNLYYAESDLDYGTNFQKRLFTKIDTIAPDEITVSSDFEARHVKLNVEERSVGPLTRKGFYLAFQDIGACVALLSVRVYYKKCPELLQGLAHFPETIAGSDAPSLATVAGTCVDHAVVPPGGEEPRMHCAVDGEWLVPIGQCLCQAGYEKVEDACQACSPGFFKFEASESPCLECPEHTLPSPEGATSCECEEGFFRAPQDPASMPCTRPPSAPHYLTAVGMGAKVELRWTPPQDSGGREDIVYSVTCEQCWPESGECGPCEASVRYSEPPHGLTRTSVTVSDLEPHMNYTFTVEARNGVSGLVTSRSFRTASVSINQTEPPKVRLEGRSTTSLSVSWSIPPPQQSRVWKYEVTYRKKGDSNSYNVRRTEGFSVTLDDLAPDTTYLVQVQALTQEGQGAGSKVHEFQTLSPEGSGNLAVIGGVAVGVVLLLVLAGVGFFIHRRRKNQRARQSPEDVYFSKSEQLKPLKTYVDPHTYEDPNQAVLKFTTEIHPSCVTRQKVIGAGEFGEVYKGMLKTSSGKKEVPVAIKTLKAGYTEKQRVDFLGEAGIMGQFSHHNIIRLEGVISKYKPMMIITEYMENGALDKFLREKDGEFSVLQLVGMLRGIAAGMKYLANMNYVHRDLAARNILVNSNLVCKVSDFGLSRVLEDDPEATYTTSGGKIPIRWTAPEAISYRKFTSASDVWSFGIVMWEVMTYGERPYWELSNHEVMKAINDGFRLPTPMDCPSAIYQLMMQCWQQERARRPKFADIVSILDKLIRAPDSLKTLADFDPRVSIRLPSTSGSEGVPFRTVSEWLESIKMQQYTEHFMAAGYTAIEKVVQMTNDDIKRIGVRLPGHQKRIAYSLLGLKDQVNTVGIPI

SEQ ID NO: 4 - DNA, 3924 bp, Homo sapiens
agggcatgaatgaacaggagtcggttctcacccaacttccattaaggactcggggcaggaggggcagaagttgcgcgcaggccggcgggcgggagcggacaccgaggccggcgtgcaggcgtgcgggtgtgcgggagccgggctcggggggatcggaccgagagcgagaagcgcggcatggagctccaggcagcccgcgcctgcttcgccctgctgtggggctgtgcgctggccgcggccgcggcggcgcagggcaaggaagtgggacctgatgcagaacatcatgaatgacatgccgatctacatgtactccgtgtgcaacgtgatgtctggcgaccaggacaactggctccgcaccaactgggtgtaccgaggagaggctgagcgtatcttcattgagctcaagtttactgtacgtgactgcaacagcttccctggtggcgccagctcctgcaaggagactttcaacctctactatgccgagtcggacctggactacggcaccaacttccagaagcgcctgttcaccaagattgacaccattgcgcccgatgagatcaccgtcagcagcgacttcgaggcacgccacgtgaagctgaacgtggaggagcgctccgtggggccgctcacccgcaaaggcttctacctggccttccaggatatcggtgcctgtgtggcgctgctctccgtccgtgtctactacaagaagtgccccgagctgctgcagggcctggcccacttccctgagaccatcgccggctctgatgcaccttccctggccactgtggccggcacctgtgtggaccatgccgtggtgccaccggggggtgaagagccccgtatgcactgtgcagtggatggcgagtggctggtgcccattgggcagtgcctgtgccaggcaggctacgagaaggtggaggatgcctgccaggcctgctcgcctggattttttaagtttgaggcatctgagagcccctgcttggagtgccctgagcacacgctgccatcccctgagggtgccacctcctgcgagtgtgaggaaggcttcttccgggcacctcaggacccagcgtcgatgccttgcacacgacccccctccgccccacactacctcacagccgtgggcatgggtgccaaggtggagctgcgctggacgccccctcaggacagcgggggccgcgaggacattgtctacagcgtcacctgcgaacagtgctggcccgagtctggggaatgcgggccgtgtgaggccagtgtgcgctactcggagcctcctcacggactgacccgcaccagtgtgacagtgagcgacctggagccccacatgaactacaccttcaccgtggaggcccgcaatggcgtctcaggcctggtaaccagccgcagcttccgtactgccagtgtcagcatcaaccagacagagccccccaaggtgaggctggagggccgcagcaccacctcgcttagcgtctcctggagcatccccccgccgcagcagagccgagtgtggaagtacgaggtcacttaccgcaagaagggagactccaacagctacaatgtgcgccgcaccgagggtttctccgtgaccctggacgacctggccccagacaccacctacctggtccaggtgcaggcactgacgcaggagggccagggggccggcagcaaggtgcacgaattccagacgctgtccccggagggatctggcaacttggcggtgattggcggcgtggctgtcggtgtggtcctgcttctggtgctggcaggagttggcttctttatccaccgcaggaggaagaaccagcgtgcccgccagtccccggaggacgtttacttctccaagtcagaacaactgaagcccctgaagacatacgtggacccccacacatatgaggaccccaaccaggctgtgttgaagttcactaccgagatccatccatcctgtgtcactcggcagaaggtgatcggagcaggagagtttggggaggtgtacaagggcatgctgaagacatcctcggggaagaaggaggtgccggtggccatcaagacgctgaaagccggctacacagagaagcagcgagtggacttcctcggcgaggccggcatcatgggccagttcagccaccacaacatcatccgcctagagggcgtcatctccaaatacaagcccatgatgatcatcactgagtacatggagaatggggccctggacaagttccttcgggagaaggatggcgagttcagcgtgctgcagctggtgggcatgctgcggggcatcgcagctggcatgaagtacctggccaacatgaactatgtgcaccgtgacctggctgcccgcaacatcctcgtcaacagcaacctggtctgcaaggtgtctgactttggcctgtcccgcgtgctggaggacgaccccgaggccacctacaccaccagtggcggcaagatccccatccgctggaccgccccggaggccatttcctaccggaagttcacctctgccagcgacgtgtggagctttggcattgtcatgtgggaggtgatgacctatggcgagcggccctactgggagttgtccaaccacgaggtgatgaaagccatcaatgatggcttccggctccccacacccatggactgcccctccgccatctaccagctcatgatgcagtgctggcagcaggagcgtgcccgccgccccaagttcgctgacatcgtcagcatcctggacaagctcattcgtgcccctgactccctcaagaccctggctgactttgacccccgcgtgtctatccggctccccagcacgagcggctcggagggggtgcccttccgcacggtgtccgagtggctggagtccatcaagatgcagcagtatacggagcacttcatggcggccggctacactgccatcgagaaggtggtgcagatgaccaacgacgacatcaagaggattggggtgcggctgcccggccaccagaagcgcatcgcctacagcctgctgggactcaaggaccaggtgaacactgtggggatccccatctgagcctcgacagggcctggagccccatcggccaagaatacttgaagaaacagagtggcctccctgctgtgccatgctgggccactggggactttatttatttctagttctttcctccccctgcaacttccgctgaggggtctcggatgacaccctggcctgaactgaggagatgaccagggatgctgggctgggccctctttccctgcgagacgcacacagctgagcacttagcaggcaccgccacgtcccagcatccctggagcaggagccccgccacagccttcggacagacatatgggatattcccaagccgaccttccctccgccttctcccacatgaggccatctcaggagatggagggcttggcccagcgccaagtaaacagggtacctcaagccccatttcctcacactaagagggcagactgtgaacttgactgggtgagacccaaagcggtccctgtccctctagtgccttctttagaccctcgggccccatcctcatccctgactggccaaacccttgctttcctgggcctttgcaagatgcttggttgtgttgaggtttttaaatatatattttgtactttgtggagagaatgtgtgtgtgtggcagggggccccgccagggctggggacagagggtgtcaaacattcgtgagctggggactcagggaccggtgctgcaggagtgtcctgcccatgccccagtcggccccatctctcatccttttggataagtttctattctgtcagtgttaaagattttgttttgttggacatttttttcgaatcttaatttattattttttttatatttattgttagaaaatgacttatttctgctctggaataaagttgcagatgattcaaaccgaaaaaaa

SEQ ID NO: 5 - protein, 1203 aa, Homo sapiens
MDKLPPSMRKRLYSLPQQVGAKAWIMDEEEDAEEEGAGGRQDPSRRSIRLRPLPSPSPSAAAGGTESRSSALGAADSEGPARGAGKSSTNGDCRRFRGSLASLGSRGGGSGGTGSGSSHGHLHDSAEERRLIAEGDASPGEDRTPPGLAAEPERPGASAQPAASPPPPQQPPQPASASCEQPSVDTAIKVEGGAAAGDQILPEAEVRLGQAGFMQRQFGAMLQPGVNKFSLRMFGSQKAVEREQERVKSAGFWIIHPYSDFRFYWDLTMLLLMVGNLIIIPVGITFFKDENTTPWIVFNVVSDTFFLIDLVLNFRTGIVVEDNTEIILDPQRIKMKYLKSWFMVDFISSIPVDYIFLIVETRIDSEVYKTARALRIVRFTKILSLLRLLRLSRLIRYIHQWEEIFHMTYDLASAVVRIVNLIGMMLLLCHWDGCLQFLVPMLQDFPDDCWVSINNMVNNSWGKQYSYALFKAMSHMLCIGYGRQAPVGMSDVWLTMLSMIVGATCYAMFIGHATALIQSLDSSRRQYQEKYKQVEQYMSFHKLPPDTRQRIHDYYEHRYQGKMFDEESILGELSEPLREEIINFNCRKLVASMPLFANADPNFVTSMLTKLRFEVFQPGDYIIREGTIGKKMYFIQHGVVSVLTKGNKETKLADGSYFGEICLLTRGRRTASVRADTYCRLYSLSVDNFNEVLEEYPMMRRAFETVALDRLDRIGKKNSILLHKVQHDLNSGVFNYQENEIIQQIVQHDREMAHCAHRVQAAASATPTPTPVIWTPLIQAPLQAAAATTSVAIALTHHPRLPAAIFRPPPGSGLGNLGAGQTPRHLKRLQSLIPSALGSASPASSPSQVDTPSSSSFHIQQLAGFSAPAGLSPLLPSSSSSPPPGACGSPSAPTPSAGVAATTIAGFGHFHKALGGSLSSSDSPLLTPLQPGARSPQAAQPSPAPPGARGGLGLPEHFLPPPPSSRSPSSSPGQLGQPPGELSLGLATGPLSTPETPPRQPEPPSLVAGASGGASPVGFTPRGGLSPPGHSPGPPRTFPSAPPRASGSHGSLLLPPASSPPPPQVPQRRGTPPLTPGRLTQDLKLISASQPALPQDGAQTLRRASPHSSGESMAAFPLFPRAGGGSGGSGSSGGLGPPGRPYGAIPGQHVTLPRKTSSGSLPPPLSLFGARATSSGGPPLTAGPQREPGARPEPVRSKLPSNL

SEQ ID NO: 6 - DNA, 7245 bp, Homo sapiens
caaaaatgccagggaaaggcgagcccagagcttggtgatggagaaattgggaagccaccccccacccttcaatcttaggatggggaattcgcaactgaagccggagcttcagacttggggcgcactcccagcttagcccaggaaagagatttaagggcgcagcagtgtggatacctctcaccccggccccgaaggtctagcgagggtctaacctgggccccttgccaggcccgccccccgcccctttccagcccccggcccgtgcgccgctgcccctttaagaagcccaggtaggcaggcccggctgctggagccgctcctatggcaacccgcgagctgcggcggcttcatgaatattccggggcgcgggagcccgagcgctgccggagggcgcttcgggggaggcggccgctgatgtaagcccggcgggtcgctgggctccgctcggttgcggcgggagccccgggacgggccggacgggccggggcagaggaggcgaggcgagctcgcgggtggccagccacaaagcccgggcggcgagacagacggacagccagccctcccgcgggacgcacgcccgggacccgcgcgggccgtgcgctctgcactccggagcggttccctgagcgccgcggccgcagagcctctccggccggcgcccattgttccccgcgggggcggggcgcctggagccgggcggcgcgccgcgcccctgaacgccagagggagggagggaggcaagaagggagcgcggggtccccgcgcccagccgggcccgggaggaggtgtagcgcggcgagcccggggactcggagcgggactaggatcctccccgcggcgcgcagcctgcccaagcatgggcgcctgaggctgcccccacgccggcggcaaaggacgcgtccccacgggcggactgaccggcgggcggacctggagcccgtccgcggcgccgcgctcctgcccccggcccggtccgaccccggcccctggcgccatggacaagctgccgccgtccatgcgcaagcggctctacagcctcccgcagcaggtgggggccaaggcgtggatcatggacgaggaagaggacgccgaggaggagggggccgggggccgccaagaccccagccgcaggagcatccggctgcggccactgccctcgccctccccctcggcggccgcgggtggcacggagtcccggagctcggccctcggggcagcggacagcgaagggccggcccgcggcgcgggcaagtccagcacgaacggcgactgcaggcgcttccgcgggagcctggcctcgctgggcagccggggcggcggcagcggcggcacggggagcggcagcagtcacggacacctgcatgactccgcggaggagcggcggctcatcgccgagggcgacgcgtcccccggcgaggacaggacgcccccaggcctggcggccgagcccgagcgccccggcgcctcggcgcagcccgcagcctcgccgccgccgccccagcagccaccgcagccggcctccgcctcctgcgagcagccctcggtggacaccgctatcaaagtggagggaggcgcggctgccggcgaccagatcctcccggaggccgaggtgcgcctgggccaggccggcttcatgcagcgccagttcggggccatgctccaacccggggtcaacaaattctccctaaggatgttcggcagccagaaagccgtggagcgcgaacaggagagggtcaagtcggccggattttggattatccacccctacagtgacttcagattttactgggacctgaccatgctgctgctgatggtgggaaacctgattatcattcctgtgggcatcaccttcttcaaggatgagaacaccacaccctggattgtcttcaatgtggtgtcagacacattcttcctcatcgacttggtcctcaacttccgcacagggatcgtggtggaggacaacacagagatcatcctggacccgcagcggattaaaatgaagtacctgaaaagctggttcatggtagatttcatttcctccatccccgtggactacatcttcctcattgtggagacacgcatcgactcggaggtctacaagactgcccgggccctgcgcattgtccgcttcacgaagatcctcagcctcttacgcctgttacgcctctcccgcctcattcgatatattcaccagtgggaagagatcttccacatgacctacgacctggccagcgccgtggtgcgcatcgtgaacctcatcggcatgatgctcctgctctgccactgggacggctgcctgcagttcctggtacccatgctacaggacttccctgacgactgctgggtgtccatcaacaacatggtgaacaactcctgggggaagcagtactcctacgcgctcttcaaggccatgagccacatgctgtgcatcggctacgggcggcaggcgcccgtgggcatgtccgacgtctggctcaccatgctcagcatgatcgtgggtgccacctgctacgccatgttcattggccacgccactgccctcatccagtccctggactcctcccggcgccagtaccaggaaaagtacaagcaggtggagcagtacatgtcctttcacaagctcccgcccgacacccggcagcgcatccacgactactacgagcaccgctaccagggcaagatgttcgacgaggagagcatcctgggcgagctaagcgagcccctgcgggaggagatcatcaactttaactgtcggaagctggtggcctccatgccactgtttgccaatgcggaccccaacttcgtgacgtccatgctgaccaagctgcgtttcgaggtcttccagcctggggactacatcatccgggaaggcaccattggcaagaagatgtacttcatccagcatggcgtggtcagcgtgctcaccaagggcaacaaggagaccaagctggccgacggctcctactttggagagatctgcctgctgacccggggccggcgcacagccagcgtgagggccgacacctactgccgcctctactcgctgagcgtggacaacttcaatgaggtgctggaggagtaccccatgatgcgaagggccttcgagaccgtggcgctggaccgcctggaccgcattggcaagaagaactccatcctcctccacaaagtccagcacgacctcaactccggcgtcttcaactaccaggagaatgagatcatccagcagattgtgcagcatgaccgggagatggcccactgcgcgcaccgcgtccaggctgctgcctctgccaccccaacccccacgcccgtcatctggaccccgctgatccaggcaccactgcaggctgccgctgccaccacttctgtggccatagccctcacccaccaccctcgcctgcctgctgccatcttccgccctcccccaggatctgggctgggcaacctcggtgccgggcagacgccaaggcacctgaaacggctgcagtccctgatcccttctgcgctgggctccgcctcgcccgccagcagcccgtcccaggtggacacaccgtcttcatcctccttccacatccaacagctggctggattctctgcccccgctggactgagcccactcctgccctcatccagctcctccccaccccccggggcctgtggctccccctcggctcccacaccatcagctggcgtagccgccaccaccatagccgggtttggccacttccacaaggcgctgggtggctccctgtcctcctccgactctcccctgctcaccccgctgcagccaggcgcccgctccccgcaggctgcccagccatctcccgcgccacccggggcccggggaggcctgggactcccggagcacttcctgccacccccaccctcatccagatccccgtcatctagccccgggcagctgggccagcctcccggggagttgtccctaggtctggccactggcccactgagcacgccagagacacccccacggcagcctgagccgccgtcccttgtggcaggggcctctgggggggcttcccctgtaggctttactccccgaggaggtctcagcccccctggccacagcccaggccccccaagaaccttcccgagtgccccgccccgggcctctggctcccacggatccttgctcctgccacctgcatccagccccccaccaccccaggtcccccagcgccggggcacacccccgctcacccccggccgcctcacccaggacctcaagctcatctccgcgtctcagccagccctgcctcaggacggggcgcagactctccgcagagcctccccgcactcctcaggggagtccatggctgccttcccgctcttccccagggctgggggtggcagcgggggcagtgggagcagcgggggcctcggtccccctgggaggccctatggtgccatccccggccagcacgtcactctgcctcggaagacatcctcaggttctttgccaccccctctgtctttgtttggggcaagagccacctcttctggggggccccctctgactgctggaccccagagggaacctggggccaggcctgagccagtgcgctccaaactgccatccaatctatgagctgggcccttccttccctcttctttcttcttttctctcccttccttcttccttcaggtttaactgtgattaggagatataccaataacagtaataattatttaaaaaaccacacacaccagaaaaacaaaagacagcagaaaataaccaggtattcttagagctatagatttttggtcacttgcttttatagactattttaatactcagcactagagggagggagggggagggaggagggagcaggcaggtcccaaatgcaaaagccagagaaaggcagatggggtctccggggctgggcaggggtgggagtggccagtgttggcggttcttagagcagatgtgtcattgtgttcatttagagaaacagctgccatcagcccgttagctgtaacttggagctccactctgcccccagaaaggggctgccctggggtgtgccctggggagcctcagaagcctgcgaccttgggagaaaagggccagggccctgagggcctagcattttttctactgtaaacgtagcaagatctgtatatgaatatgtatatgtatatgtatgtaagatgtgtatatgtatagctatgtagcgctctgtagagccatgtagatagccactcacatgtgcgcacacgtgtgcggtctagtttaatcccatgttgacaggatgcccaggtcaccttacacccagcaacccgccttggcccacaggctgtgcactgcatggtctagggacgttctctctccagtcctcagggaagaggaccccaggacttcgcagcaggccccctctctccccatctctggtctcaaagccagtcccagcctgacctctcaccacacggaagtggaagactcccctttcctagggcctcaagcacacaccgccacctctggggccgtcagtttgcccatctgtacagtgggaggtgagcggaacttctgtttattgagtctgctctgtgccaagcactggtttcgcactttacacacattaactccttcagtttcacaaagaccatggggtgggtactttgattctccccatttagcagaggaagaaacagttttgggtaatttttccagaatcatgtaactaggagtggcagagtggggactgatttgaggttcgagtccacgcctccttgaggcccaagtctgtgttccttccatcagaaaactgtgttgaggggggctgaggtagatggtccccaagcatggtacagaaggaagacaccagattttggcagcagtcaggcctgggtttgaatcccagccctgccacttcttagctgtatgatcttgggcaagttatctgacctttctgtcacctcatttgtaaaatgggaataattatggtactgcctcacaaggacctatgaggaccagatgagaaaaatctatatgtgaaatgcccagcccagcgcctggcacataccatggtaggtgctcaataaaaaatcacatttcttctgcccctcatatgcccagcctattgctccagcaaactatgtgagagcccagggagctttggctgagggctccaagacttaaaatctcaggactcaggaggtggctgggcctccctaagggcccaaggaaggtgtgtggccagaggtgggtgggagccaggccttgagaagtgggaagacttcaacagggagagagggagggaatggtgggtgggatggagtgtatggtggggagattcctgaggtggatgtggagtggtggatcagggctttgggaggggatccccaggctgaggggtcagagggacggccttgggtgatagggtaagggattgtctgggcttagtcctggcaactaggagccataagcaggttccagattgcgggaacgagaaagcagctcagatgcctttggaggcaccatcctccctcctcccagatgggatcttgccagagccaaggtcaggggtctgcccctgcctatagggccagagcaggtatggctgcaatccccaagtaatgagaagggctggtcccacattatccatccagaaccttccatgctccaagccagaatgttggcaagatcgggttttgccttgagctatcctgggatgtgagacaaaccgatttctccatagatgggctgcagggagtgggaggcagtactccaggagagaagtgggtgaaggttcctgggatcttaggtaaagactagacgccgcctagtactggtctctactgtgctggctcaggagttctgagaactggaaggacttagcctcaacctgagttctgcacacaccccttccccttaaggaaggcagctctgagaggcagcaggacttgatccaaacccacagtcttgtcctggaggcagcaggggtgaaggtggagggtccagggccatgaggagcccccttgccatcagagcctggcctaaccaccctcttctctacttacacacacatgcattttataatagctctgacccaacctggccactctgcagagactgggacagacaggtgcaggcaatgggccctcccacacccagtcacctacaaggaattttcaaatccacttttaaaacagaaaccggtaaatgcgccgtattgtatattttatttaaataaaaaaaattccagcaaaaaaaaaaaaaaaaaa

SEQ ID NO: 7 - protein, 349 aa, Homo sapiens
MGDMGDPPKKKRLISLCVGCGNQIHDQYILRVSPDLEWHAACLKCAECNQYLDESCTCFVRDGKTYCKRDYIRLYGIKCAKCSIGFSKNDFVMRARSKVYHIECFRCVACSRQLIPGDEFALREDGLFCRADHDVVERASLGAGDPLSPLHPARPLQMAAEPISARQPALRPHVHKQPEKTTRVRTVLNEKQLHTLRTCYAANPRPDALMKEQLVEMTGLSPRVIRVWFQNKRCKDKKRSIMMKQLQQQQPNDKTNIQGMTGTPMVAASPERHDGGLQANPVEVQSYQPPWKVLSDFALQSDIDQPAFQQLVNFSEGGPGSNSTGSEVASMSSQLPDTPNSMVASPIEA

SEQ ID NO: 8 - DNA, 2729 bp, Homo sapiens
gaaggaagaggaagaggaggagagggaggccagagccagaacagcccggcagcccgagcttcgggggagaacggcctgagccccgagcaagttgcctcgggagccctaatcctctcccgctggctcgccgagcggtcagtggcgctcagcggcggcgaggctgaaatatgataatcagaacagctgcgccgcgcgccctgcagccaatgggcgcggcgctcgcctgacgtccccgcgcgctgcgtcagaccaatggcgatggagctgagttggagcagagaagtttgagtaagagataaggaagagaggtgcccgagccgcgccgagtctgccgccgccgcagcgcctccgctccgccaactccgccggcttaaattggactcctagatccgcgagggcgcggcgcagccgagcagcggctctttcagcattggcaaccccaggggccaatatttcccacttagccacagctccagcatcctctctgtgggctgttcaccaactgtacaaccaccatttcactgtggacattactccctcttacagatatgggagacatgggagatccaccaaaaaaaaaacgtctgatttccctatgtgttggttgcggcaatcagattcacgatcagtatattctgagggtttctccggatttggaatggcatgcggcatgtttgaaatgtgcggagtgtaatcagtatttggacgagagctgtacatgctttgttagggatgggaaaacctactgtaaaagagattatatcaggttgtacgggatcaaatgcgccaagtgcagcatcggcttcagcaagaacgacttcgtgatgcgtgcccgctccaaggtgtatcacatcgagtgtttccgctgtgtggcctgcagccgccagctcatccctggggacgaatttgcgcttcgggaggacggtctcttctgccgagcagaccacgatgtggtggagagggccagtctaggcgctggcgacccgctcagtcccctgcatccagcgcggccactgcaaatggcagcggagcccatctccgccaggcagccagccctgcggccccacgtccacaagcagccggagaagaccacccgcgtgcggactgtgctgaacgagaagcagctgcacaccttgcggacctgctacgccgcaaacccgcggccagatgcgctcatgaaggagcaactggtagagatgacgggcctcagtccccgtgtgatccgggtctggtttcaaaacaagcggtgcaaggacaagaagcgaagcatcatgatgaagcaactccagcagcagcagcccaatgacaaaactaatatccaggggatgacaggaactcccatggtggctgccagtccagagagacacgacggtggcttacaggctaacccagtggaagtacaaagttaccagccaccttggaaagtactgagcgacttcgccttgcagagtgacatagatcagcctgcttttcagcaactggtcaatttttcagaaggaggaccgggctctaattccactggcagtgaagtagcatcaatgtcctctcaacttccagatacacctaacagcatggtagccagtcctattgaggcatgaggaacattcattctgtattttttttccctgttggagaaagtgggaaattataatgtcgaactctgaaacaaaagtatttaacgacccagtcaatgaaaactgaatcaagaaatgaatgctccatgaaatgcacgaagtctgttttaatgacaaggtgatatggtagcaacactgtgaagacaatcatgggattttactagaattaaacaacaaacaaaacgcaaaacccagtatatgctattcaatgatcttagaagtactgaaaaaaaaagacgtttttaaaacgtagaggatttatattcaaggatctcaaagaaagcattttcatttcactgcacatctagagaaaaacaaaaatagaaaattttctagtccatcctaatctgaatggtgctgtttctatattggtcattgccttgccaaacaggagctccagcaaaagcgcaggaagagagactggcctccttggctgaaagagtcctttcaggaaggtggagctgcattggtttgatatgtttaaagttgactttaacaaggggttaattgaaatcctgggtctcttggcctgtcctgtagctggtttattttttactttgccccctccccactttttttgagatccatcctttatcaagaagtctgaagcgactataaaggtttttgaattcagatttaaaaaccaacttataaagcattgcaacaaggttacctctattttgccacaagcgtctcgggattgtgtttgacttgtgtctgtccaagaacttttcccccaaagatgtgtatagttattggttaaaatgactgttttctctctctatggaaataaaaaggaaaaaaaaaaaggaaactttttttgtttgctcttgcattgcaaaaattataaagtaatttattatttattgtcggaagacttgccacttttcatgtcatttgacattttttgtttgctgaagtgaaaaaaaaagataaaggttgtacggtggtctttgaattatatgtctaattctatgtgttttgtctttttcttaaatattatgtgaaatcaaagcgccatatgtagaattatatcttcaggactatttcactaataaacatttggcatagataaataaataaaaaaaaaaaaaa

SEQ ID NO: 9 - protein, 5 aa, Artificial Sequence
  Description of Artificial Sequence Synthetic peptide
  MOD_RES (4)..(4): S or T
PPPXP

(19) United States
(12) Patent Application Publication
(10) Pub. No.: US 2022/0073883 A1
(43) Pub. Date: Mar. 10, 2022

(54) COMPOSITIONS AND METHODS FOR GENERATION OF HEART FIELD-SPECIFIC PROGENITOR CELLS

(71) Applicant: The Johns Hopkins University, Baltimore, MD (US)

(72) Inventors: Peter Andersen, Baltimore, MD (US); Chulan Kwon, Ellicott City, MD (US)

(21) Appl. No.: 17/263,142

(22) PCT Filed: Jul. 25, 2019
(86) PCT No.: PCT/US2019/043537

§ 371 Date: Jan. 25, 2021

Related U.S. Application Data
(60) Provisional application No. 62/703,425, filed on Jul. 25, 2018.

(51) Int. Cl. C12N 5/077; C12N 5/074; A61K 35/34; A61P 9/00; A61K 35/28

(52) U.S. Cl. CPC C12N 5/0657; C12N 5/0696; A61K 35/34; A61P 9/00; A61K 35/28; C12N 2501/21; C12N 2501/415; C12N 2501/155; C12N 2501/16; C12N 2506/45; C12N 2501/15

(57) ABSTRACT

The present invention relates to compositions and methods for producing, identifying and isolating heart progenitor cells.

The present application claims the benefit of U.S. provisional application 62/703,425, filed Jul. 25, 2018, which is incorporated by reference herein in its entirety.

### STATEMENT AS TO FEDERALLY SPONSORED RESEARCH

This invention was made with government support under 2013-MSCRFF-0066 awarded by the Maryland Stem Cell Research Fund and by grants from NHLBI/NIH (R01IL111198), NICHD/NIH (R01HD086026), MSCRF (2015-MSCRFI-1622). The government has certain rights in the invention.

### BACKGROUND

Over the past few decades, major advances have been made in identifying the origins of cardiac cells from developing embryos. In particular, the discovery of the first heart field (FHF) and the second heart field (SHF) led to an understanding of how diverse lineages and structures of the heart arise during cardiogenesis. However, it remains unknown how the two heart fields are specified and segregated, a fundamental step toward understanding heart formation and developing pluripotent stem cell (PSC)-based therapeutic strategies. As such, there was a pressing need to develop methods for selective induction of heart field populations, which allow modeling, drug testing, and treating heart diseases.

### SUMMARY

The invention is based, at least in part, on the understanding and identification that Bmp and Wnt are among the most differentially regulated pathways in the development of two heart field populations (FHF and SHF). Gain- and loss-of-function studies showed that Bmp signaling specifies FHF cells and SHF cells via the Bmp/Smad pathway and Wnt signaling, respectively.

Furthermore, SHF cells were distinguished and isolated by the surface protein Cxcr4. The invention herein provides fundamental insights into understanding the specification of the two cardiac origins, which can be leveraged to generate heart field-specific progenitors for PSC-based modeling of heart field/chamber-specific disease.

Accordingly, in certain embodiments, a method of producing first heart field (FHF) induced pluripotent stem (iPS) cell or second heart field (SHF) iPS, comprises providing a population of pluripotent stem cells from a mammal, wherein a cluster of cells is formed, contacting the cluster of cells with one or more reprogramming factors, thereby producing FHF iPS cells or SHF iPS cells.

In certain embodiments, the one or more reprogramming factors comprises a transforming growth factor beta (TGF-β) protein, a Wnt protein, or a bone morphogenic protein (Bmp). In certain embodiments the Wnt protein comprises Wnt3A, Wnt5A or Wnt11, the Bmp protein comprises Bmp4, the TGF-β protein comprises Activin A, or any combinations thereof.

In certain embodiments, the method further comprises providing a Wnt pathway activator, comprising a glycogen synthesis kinase 3 (Gsk3) inhibitor.

In certain embodiments, the iPSC is genetically modified to alter the expression or activity of C-X-C chemokine receptor type 4 (Cxcr4), and thereby producing SHF iPS cells. In certain embodiments, the cells are mammalian cells, wherein the mammal is selected from a group consisting of: rodents, rats, mice, rabbits, goats, non-human primates, humans, dogs, bears, cats, lions, tigers, elephants, llamas, donkeys, mules, bovines, ovines, pigs, and horses. In certain embodiments the iPS cells are human iPS (hiPS) cells.

In certain embodiments, a method of treating a disease or condition comprises administering to a subject, the iPS cells produced by the methods embodied herein.

In certain embodiments, a cell comprises an agent that alters the expression or activity of C-X-C chemokine receptor type 4 (Cxcr4), wherein the cell comprises a genetically modified stem cell, mesenchymal stem cell, induced pluripotent stem cell (iPSC), iPSC-derived pericytes, or iPSC-derived cardiac muscle cell. In ceratine embodiments, the agent is selected from the group consisting of an antibody or fragment thereof, a peptide, a polypeptide or fragments thereof, a small molecule, and a nucleic acid.

In certain embodiments a pharmaceutical composition comprises a genetically modified stem cell, mesenchymal stem cell, induced pluripotent stem cell (iPSC), iPSC-derived pericytes, or iPSC-derived cardiac muscle cell.

In certain embodiments a method for treating or preventing a heart disease in a subject, comprises administering a genetically modified stem cell, mesenchymal stem cell, induced pluripotent stem cell (iPSC), iPSC-derived pericytes, or iPSC-derived cardiac muscle cell to the subject. In certain embodiments, the stem cell, the mesenchymal stem cell or the iPSC is derived from the subject. In certain aspects the stem cell or the iPSC has been genetically modified to alter the expression or activity of C-X-C chemokine receptor type 4 (Cxcr4). In one aspect, the heart disease comprises a congenital heart disease (CHD).

In certaine embodiments, a composition comprises induced pluripotent stem cells comprising a vector encoding C-X-C chemokine receptor type 4 (Cxcr4).

Furthermore, as described herein, adult cells produced by the methods provided herein are used to treat diseases or conditions, such as those that manifest later in life. In some embodiments, adult cells generated by the methods described herein are used to model and treat diseases or conditions in a subject such as cardiac-related disorders (e.g., congenital heart defect (CHD)).

The subject is preferably a mammal in need of such treatment, e.g., a subject that has been diagnosed with a cardiac-related disease or a predisposition thereto. The mammal is any mammal, e.g., a human, a primate, a mouse, a rat, a dog, a cat, a horse, as well as livestock or animals grown for food consumption, e.g., cattle, sheep, pigs, chickens, and goats. In a preferred embodiment, the mammal is a human.

In some cases, the disease consists of a cardiac disorder. Exemplary cardiac disorders include cardiovascular disease, cardiomyopathy, atherosclerosis, myocardial infarction, stroke, endocarditis, rheumatic heart disease, hypertensive heart disease, and angina. In preferred embodiments, the cardiac disorder is a congenital heart defect (CHD). In embodiments, the CHD includes hypoplastic left heart syndrome and hypoplastic right heart syndrome as well as some chamber specific cardiomyopathies and tachyarrhythmias like arrhythmogenic right ventricular cardiomyopathy or right ventricular outflow track ventricular tachycardia.

In some cases, during the treatment methods described herein, the composition is administered to a subject in need thereof via oral administration, intravenous administration, topical administration, parenteral administration, intraperitoneal administration, intramuscular administration, intrathecal administration, intralesional administration, intracranial administration, intranasal administration, intraocular administration, intracardiac administration, intravitreal administration, intraosseous administration, intracerebral administration, intraarterial administration, intraarticular administration, intradermal administration, transdermal administration, transmucosal administration, sublingual administration, enteral administration, sublabial administration, insufflation administration, suppository administration, inhaled administration, intraventricular injection, injection into the brain or spinal cord, or subcutaneous administration.

### Definitions

Unless defined otherwise, all technical and scientific terms used herein have the meaning commonly understood by a person skilled in the art to which this invention belongs. The following references provide one of skill with a general definition of many of the terms used in this invention: The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “agent” is meant any small compound, antibody, nucleic acid molecule, protein, or polypeptide, or fragments thereof that alters the expression or activity of a protein.

By “alteration” is meant a change (increase or decrease) in the expression levels or activity of a gene or polypeptide as detected by standard art known methods such as those described herein. As used herein, an alteration includes at least a 1% change in expression levels, e.g., at least a 2%, 3%, 4%, 5%, 6%, 7%, 8%, 9%, 10%, 20%, 30%, 40%, 50%, 60%, 70%, 80%, 90%, or 100% change in expression levels. For example, an alteration includes at least a 5%-10% change in expression levels, preferably a 25% change, more preferably a 40% change, and most preferably a 50% or greater change in expression levels.

As used herein an “alteration” also includes a 2-fold or more change in expression levels or activity of a gene or polypeptide, for example, 5-fold, 10-fold, 20-fold, 30-fold, 40-fold, 50-fold, 100-fold, 500-fold, 1000-fold or more.

By “ameliorate” is meant decrease, suppress, attenuate, diminish, arrest, or stabilize the development or progression of a disease.

By “binding to” a molecule is meant having a physicochemical affinity for that molecule.

The term “pharmaceutical composition” is meant any composition, which contains at least one therapeutically or biologically active agent and is suitable for administration to the patient. Any of these formulations can be prepared by well-known and accepted methods of the art. See, for example, Remington: The Science and Practice of Pharmacy, 20th edition, (ed. A. R. Gennaro), Mack Publishing Co., Easton, Pa., 2000.

In this disclosure, “comprises,” “comprising,” “containing,” “having,” and the like can have the meaning ascribed to them in U.S. patent law and can mean “includes,” “including,” and the like; the terms “consisting essentially of” or “consists essentially” likewise have the meaning ascribed in U.S. patent law and these terms are open-ended, allowing for the presence of more than that which is recited so long as basic or novel characteristics of that which is recited are not changed by the presence of more than that which is recited, but excludes prior art embodiments.

By “effective amount” is meant the amount required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

The terms “treating” and “treatment” as used herein refer to the administration of an agent or formulation to a clinically symptomatic individual afflicted with an adverse condition, disorder, or disease, so as to effect a reduction in severity and/or frequency of symptoms, eliminate the symptoms and/or their underlying cause, and/or facilitate improvement or remediation of damage.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

By “recombinant” is meant nucleic acid molecules formed by laboratory methods of genetic recombination (such as molecular cloning) to bring together genetic material from multiple sources, creating sequences that would not otherwise be found in biological organisms.

A “heterologous promoter” is a promoter which is different from the promoter to which a gene or nucleic acid sequence is operably linked in nature. The term “operably linked” refers to functional linkage between a nucleic acid expression control sequence (such as a promoter, signal sequence, or array of transcription factor binding sites) and a second nucleic acid sequence, wherein the expression control sequence affects transcription and/or translation of the nucleic acid corresponding to the second sequence. A “heterologous polynucleotide” or a “heterologous gene”, as used herein, is one that originates from a source foreign to the particular host cell, or, if from the same source, is modified from its original form.

By “reduces” is meant a negative alteration of at least 10%, 25%, 50%, 75%, or 100%.

By “reference” is meant a standard or control condition.

Unless specifically stated or obvious from context, as used herein, the terms “a,” “an,” and “the” are understood to be singular or plural. Unless specifically stated or obvious from context, as used herein, the term “or” is understood to be inclusive.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

By “control” or “reference” is meant a standard of comparison. As used herein, “changed as compared to a control” sample or subject is understood as having a level that is statistically different than a sample from a normal, untreated, or control sample. Control samples include, for example, cells in culture, one or more laboratory test animals, or one or more human subjects. Methods to select and test control samples are within the ability of those in the art. An analyte can be a naturally occurring substance that is characteristically expressed or produced by the cell or organism (e.g., an antibody, a protein) or a substance produced by a reporter construct (e.g, 3-galactosidase or luciferase). Depending on the method used for detection, the amount and measurement of the change can vary. Determination of statistical significance is within the ability of those skilled in the art, e.g., the number of standard deviations from the mean that constitute a positive result.

“Detect” refers to identifying the presence, absence or amount of the analyte to be detected.

As used herein, the term “diagnosing” refers to classifying pathology or a symptom, determining a severity of the pathology (e.g., grade or stage), monitoring pathology progression, forecasting an outcome of pathology, and/or determining prospects of recovery.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, 100, 200, 300, 400, 500, 600, 700, 800, 900, or 1000 nucleotides or amino acids.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” nucleic acid or protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “marker” is meant any protein or polynucleotide having an alteration in expression level or activity that is associated with a disease or disorder.

By “modulate” is meant alter (increase or decrease). Such alterations are detected by standard art known methods such as those described herein.

The term, “normal amount” refers to a normal amount of a complex in an individual known not to be diagnosed with a disease or disorder. The amount of the molecule can be measured in a test sample and compared to the “normal control level,” utilizing techniques such as reference limits, discrimination limits, or risk defining thresholds to define cutoff points and abnormal values (e.g., for cardiac disease). The “normal control level” means the level of one or more proteins (or nucleic acids) or combined protein indices (or combined nucleic acid indices) typically found in a subject known not to be suffering from prostate cancer. Such normal control levels and cutoff points may vary based on whether a molecule is used alone or in a formula combining other proteins into an index. Alternatively, the normal control level can be a database of protein patterns from previously tested subjects who did not convert to a disease or disorder over a clinically relevant time horizon.

The level that is determined may be the same as a control level or a cut off level or a threshold level, or may be increased or decreased relative to a control level or a cut off level or a threshold level. In some aspects, the control subject is a matched control of the same species, gender, ethnicity, age group, smoking status, body mass index (BMI), current therapeutic regimen status, medical history, or a combination thereof, but differs from the subject being diagnosed in that the control does not suffer from the disease in question or is not at risk for the disease.

Relative to a control level, the level that is determined may be an increased level. As used herein, the term “increased” with respect to level (e.g., expression level, biological activity level, etc.) refers to any % increase above a control level. The increased level may be at least or about a 1% increase, at least or about a 5% increase, at least or about a 10% increase, at least or about a 15% increase, at least or about a 20% increase, at least or about a 25% increase, at least or about a 30% increase, at least or about a 35% increase, at least or about a 40% increase, at least or about a 45% increase, at least or about a 50% increase, at least or about a 55% increase, at least or about a 60% increase, at least or about a 65% increase, at least or about a 70% increase, at least or about a 75% increase, at least or about a 80% increase, at least or about a 85% increase, at least or about a 90% increase, or at least or about a 95% increase, relative to a control level.

Relative to a control level, the level that is determined may be a decreased level. As used herein, the term “decreased” with respect to level (e.g., expression level, biological activity level, etc.) refers to any % decrease below a control level. The decreased level may be at least or about a 1% decrease, at least or about a 5% decrease, at least or about a 10% decrease, at least or about a 15% decrease, at least or about a 20% decrease, at least or about a 25% decrease, at least or about a 30% decrease, at least or about a 35% decrease, at least or about a 40% decrease, at least or about a 45% decrease, at least or about a 50% decrease, at least or about a 55% decrease, at least or about a 60% decrease, at least or about a 65% decrease, at least or about a 70% decrease, at least or about a 75% decrease, at least or about a 80% decrease, at least or about a 85% decrease, at least or about a 90% decrease, or at least or about a 95% decrease, relative to a control level.

The phrase “pharmaceutically acceptable carrier” is art recognized and includes a pharmaceutically acceptable material, composition or vehicle, suitable for administering compounds of the present invention to mammals. The carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

As used herein, an “antagonist” may refer to an antibody or fragment thereof, peptides, polypeptide or fragments thereof, small molecules, and inhibitory nucleic acids or fragments thereof that interferes with the activity or binding of another, for example, by competing for the one or more binding sites of an agonist, but does not induce an active response.

Alternatively, an “agonist” or activating antibody is one that enhances or initiates signaling by the antigen to which it binds. In some embodiments, agonist antibodies cause or activate signaling without the presence of the natural ligand.

An “isolated antibody,” as used herein, is intended to refer to an antibody that is substantially free of other antibodies having different antigenic specificities (e.g., an isolated antibody that specifically binds Cxcr4 and is substantially free of antibodies that specifically bind antigens other than Cxcr4). Moreover, an isolated antibody may be substantially free of other cellular material and/or chemicals.

The antibody of the present invention may be a polyclonal antisera or monoclonal antibody. The term antibody may include any of the various classes or sub-classes of immunoglobulin (e.g., IgG, IgA, IgM, IgD, or IgE derived from any animal, e.g., any of the animals conventionally used, e.g., sheep, rabbits, goats, or mice). Preferably, the antibody comprises a monoclonal antibody, e.g., a Cxcr4 monoclonal antibody.

The terms “monoclonal antibody” or “monoclonal antibody composition” as used herein refer to a preparation of antibody molecules of single molecular composition. A monoclonal antibody composition displays a single binding specificity and affinity for a particular epitope.

An “antibody fragment” comprises a portion of an intact antibody, preferably the antigen binding and/or the variable region of the intact antibody. Non-limiting examples of antibody fragments include Fab, Fab*, F(ab′)₂ and Fv fragments; diabodies; linear antibodies; single-chain antibody molecules and multispecific antibodies formed from antibody fragments.

The invention may further comprise a humanized antibody, wherein the antibody is from a non-human species, whose protein sequence has been modified to increase their similarity to antibody variants produced naturally in humans. Generally, a humanized antibody has one or more amino acid residues introduced into it from a source which is non-human. These non-human amino acid residues are referred to herein as “import” residues, which are typically taken from an “import” antibody domain, particularly a variable domain.

The term “recombinant human antibody,” as used herein, includes all human antibodies that are prepared, expressed, created or isolated by recombinant means, such as (a) antibodies isolated from animals (e.g., sheep, rabbits, goats, or mice) that are transgenic or transchromosomal for human immunoglobulin genes, (b) antibodies isolated from a host cell transformed to express the human antibody, (c) antibodies isolated from a recombinant, combinatorial human antibody library, and (d) antibodies prepared, expressed, created or isolated by any other means that involve splicing of human immunoglobulin gene sequences to other DNA sequences.

By “protein” or “polypeptide” or “peptide” is meant any chain of more than two natural or unnatural amino acids, regardless of post-translational modification (e.g., glycosylation or phosphorylation), constituting all or part of a naturally-occurring or non-naturally occurring polypeptide or peptide, as is described herein.

By, “small molecule” may be referred to broadly as an organic, inorganic or organometallic compound with a low molecular weight compound (e.g., a molecular weight of less than about 2,000 Da or less than about 1,000 Da). The small molecule may have a molecular weight of less than about 2,000 Da, a molecular weight of less than about 1,500 Da, a molecular weight of less than about 1,000 Da, a molecular weight of less than about 900 Da, a molecular weight of less than about 800 Da, a molecular weight of less than about 700 Da, a molecular weight of less than about 600 Da, a molecular weight of less than about 500 Da, a molecular weight of less than about 400 Da, a molecular weight of less than about 300 Da, a molecular weight of less than about 200 Da, a molecular weight of less than about 100 Da, or a molecular weight of less than about 50 Da.

Small molecules are organic or inorganic. Exemplary organic small molecules include, but are not limited to, aliphatic hydrocarbons, alcohols, aldehydes, ketones, organic acids, esters, mono- and disaccharides, aromatic hydrocarbons, amino acids, and lipids. Exemplary inorganic small molecules comprise trace minerals, ions, free radicals, and metabolites. Alternatively, small molecules can be synthetically engineered to consist of a fragment, or small portion, or a longer amino acid chain to fill a binding pocket of an enzyme. Typically small molecules are less than one kilodalton.

By “pluripotency” or “pluripotent stem cells” is meant stem cells with the potential to differentiate into any of the three germ layers: endoderm (interior stomach lining, gastrointestinal tract, the lungs), mesoderm (muscle, bone, blood, urogenital), or ectoderm (epidermal tissues and nervous system). However, cell pluripotency is a continuum, ranging from the completely pluripotent cell that can form every cell of the embryo proper, e.g., embyronic stem cells, to the incompletely or partially pluripotent cell that can form cells of all three germ layers, but that may not exhibit all the characteristics of completely pluripotent cells.

By “stem cells” is meant undifferentiated biological cells that can differentiate into specialized cells and can divide (e.g., through mitosis) to produce more stem cells. They are found in multicellular organisms. In mammals, there are two broad types of stem cells: embryonic stem cells, which are isolated from the inner cell mass of blastocysts, and adult stem cells, which are found in various tissues.

An “induced pluripotent stem cell” refers to a pluripotent stem cell artificially (e.g. non-naturally, in a laboratory setting) derived from a non-pluripotent cell. A “non-pluripotent cell” can be a cell of lesser potency to self-renew and differentiate than a pluripotent stem cell. Cells of lesser potency can be, but are not limited to adult stem cells, tissue specific progenitor cells, primary or secondary cells. An adult stem cell is an undifferentiated cell found throughout the body after embryonic development. Adult stem cells multiply by cell division to replenish dying cells and regenerate damaged tissue. Adult stem cells have the ability to divide and create another like cell and also divide and create a more differentiated cell. Even though adult stem cells are associated with the expression of pluripotency markers such as Rex1, Nanog, Oct4 or Sox2, they do not have the ability of pluripotent stem cells to differentiate into the cell types of all three germ layers. Adult stem cells have a limited potency to self-renew and generate progeny of distinct cell types. Without limitation, an adult stem cell can be a hematopoietic stem cell, a cord blood stem cell, a mesenchymal stem cell, an epithelial stem cell, a skin stem cell or a neural stem cell. A tissue specific progenitor refers to a cell devoid of self-renewal potential that is committed to differentiate into a specific organ or tissue. A primary cell includes any cell of an adult or fetal organism apart from egg cells, sperm cells and stem cells. Examples of useful primary cells include, but are not limited to, skin cells, bone cells, blood cells, cells of internal organs and cells of connective tissue. A secondary cell is derived from a primary cell and has been immortalized for long-lived in vitro cell culture.

Identification of the induced pluripotent stem cell may include, but is not limited to the evaluation of the aforementioned pluripotent stem cell characteristics. Such pluripotent stem cell characteristics include without further limitation, the expression or non-expression of certain combinations of molecular markers. Further, cell morphologies associated with pluripotent stem cells are also pluripotent stem cell characteristics.

The term “progenitor cell” as used herein refers to a cell that has the capacity to differentiate into a specific type of cell, as well as replicate to generate a daughter cell substantially equivalent to itself. In some instances, a progenitor cell undergoes limited self-renewal such that it does not self-replicate indefinitely.

The term “self-renewal” or “self-renewing” refers to the ability of a cell to divide through numerous cycles of cell division and generate a daughter with the same characteristics as the parent cell. The other daughter cell can have characteristics different from its parent cell. The term includes the ability of a cell to generate an identical genetic copy of itself (e.g., clone) by cell division. For example, a self-renewing cardiac progenitor cell can divide to form one daughter cardiac progenitor cell and another daughter cell committed to differentiation to a cardiac lineage such as an endothelial, smooth muscle or cardiomyocyte cell. In some instances, a self-renewing cell does not undergo cell division forever.

The term “reprogramming” refers to the process of dedifferentiating a non-pluripotent cell (e.g., an origin cell) into a cell exhibiting pluripotent stem cell characteristics (e.g., a human induced pluripotent stem cell).

The term “signaling pathway” as used herein refers to a series of interactions between cellular and optionally extra-cellular components (e.g. proteins, nucleic acids, small molecules, ions, lipids) that conveys a change in one component to one or more other components, which in turn may convey a change to additional components, which is optionally propagated to other signaling pathway components.

The term “subject” as used herein includes all members of the animal kingdom prone to suffering from the indicated disorder. In some aspects, the subject is a mammal, and in some aspects, the subject is a human. The methods are also applicable to companion animals such as dogs and cats as well as livestock such as cows, horses, sheep, goats, pigs, and other domesticated and wild animals.

A subject “suffering from or suspected of suffering from” a specific disease, condition, or syndrome has a sufficient number of risk factors or presents with a sufficient number or combination of signs or symptoms of the disease, condition, or syndrome such that a competent individual would diagnose or suspect that the subject was suffering from the disease, condition, or syndrome. Methods for identification of subjects suffering from or suspected of suffering from conditions associated with heart disease, neurodegenerative disorders, etc. is within the ability of those in the art. Subjects suffering from, and suspected of suffering from, a specific disease, condition, or syndrome are not necessarily two distinct groups.

As used herein, “susceptible to” or “prone to” or “predisposed to” or “at risk of developing” a specific disease or condition refers to an individual who based on genetic, environmental, health, and/or other risk factors is more likely to develop a disease or condition than the general population. An increase in likelihood of developing a disease may be an increase of about 10%, 20%, 50%, 100%, 150%, 200%, or more.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

The terms “treat,” treating,” “treatment,” as used herein refer to the administration of an agent or formulation to a clinically symptomatic individual afflicted with an adverse condition, disorder, or disease, so as to effect a reduction in severity and/or frequency of symptoms, eliminate the symptoms and/or their underlying cause, and/or facilitate improvement or remediation of damage. It will be appreciated that, although not precluded, treating a disorder or condition does not require that the disorder, condition or symptoms associated therewith be completely eliminated.

The terms “prevent”, “preventing”, “prevention”, “prophylactic treatment” refer to the administration of an agent or composition to a clinically asymptomatic individual who is at risk of developing, susceptible, or predisposed to a particular adverse condition, disorder, or disease, and thus relates to the prevention of the occurrence of symptoms and/or their underlying cause.

In some cases, a composition of the invention is administered orally or systemically. Other modes of administration include rectal, topical, intraocular, buccal, intravaginal, intracisternal, intracerebroventricular, intratracheal, nasal, transdermal, within/on implants, or parenteral routes. The term “parenteral” includes subcutaneous, intrathecal, intravenous, intramuscular, intraperitoneal, or infusion. Intravenous or intramuscular routes are not particularly suitable for long-term therapy and prophylaxis. They could, however, be preferred in emergency situations. Compositions comprising a composition of the invention can be added to a physiological fluid, such as blood. Oral administration can be preferred for prophylactic treatment because of the convenience to the patient as well as the dosing schedule. Parenteral modalities (subcutaneous or intravenous) may be preferable for more acute illness, or for therapy in patients that are unable to tolerate enteral administration due to gastrointestinal intolerance, ileus, or other concomitants of critical illness. Inhaled therapy may be most appropriate for pulmonary vascular diseases (e.g., pulmonary hypertension).

Pharmaceutical compositions may be assembled into kits or pharmaceutical systems for use in arresting cell cycle in rapidly dividing cells, e.g., cancer cells. Kits or pharmaceutical systems according to this aspect of the invention comprise a carrier means, such as a box, carton, tube, having in close confinement therein one or more container means, such as vials, tubes, ampoules, bottles, syringes, or bags. The kits or pharmaceutical systems of the invention may also comprise associated instructions for using the kit.

Any compositions or methods provided herein can be combined with one or more of any of the other compositions and methods provided herein.

Unless specifically stated or obvious from context, as used herein, the term “or” is understood to be inclusive. Unless specifically stated or obvious from context, as used herein, the terms “a”, “an”, and “the” are understood to be singular or plural.

In the descriptions herein and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an unrecited feature or element is also permissible.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

Other features and advantages of the invention will be apparent from the following description of the preferred embodiments thereof, and from the claims. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All published foreign patents and patent applications cited herein are incorporated herein by reference. Genbank and NCBI submissions indicated by accession number cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting.

All genes, gene names, and gene products disclosed herein are intended to correspond to homologs from any species for which the compositions and methods disclosed herein are applicable. Thus, the terms include, but are not limited to genes and gene products from humans and mice. It is understood that when a gene or gene product from a particular species is disclosed, this disclosure is intended to be exemplary only, and is not to be interpreted as a limitation unless the context in which it appears clearly indicates. Thus, for example, for the genes or gene products disclosed herein, which in some embodiments relate to mammalian nucleic acid and amino acid sequences, are intended to encompass homologous and/or orthologous genes and gene products from other animals including, but not limited to other mammals, fish, amphibians, reptiles, and birds. In preferred embodiments, the genes, nucleic acid sequences, amino acid sequences, peptides, polypeptides and proteins are human.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A-FIG. 1E depict images showing that FHF/SHF-like cells are induced in spheroid PSC culture. FIG. 1A depicts an image of live imaging of Hcn4-GFP, Tbx1-Cre, Ai9 mice E8.0. GFP is exclusively expressed in the cardiac crescent and primitive myotube, whereas RFP is expressed in the posterior region. FIG. 1B depicts an image of live imaging of Hcn4-GFP, Tbx1-Cre, Ai9 mice E9.0. GFP expression is restricted to the LV and atria whereas RFP is expressed in the pharyngeal region posterior to the heart, the outflow tract and RV. FIG. 1C is a schematic of the strategy used to generate and differentiate ESC-derived cardiac spheroids. FIG. 1D is an image depicting flow cytometric analyses of Hcn4-GFP⁺ and Tbx1-Cre, RFP⁺ in cardiac spheroids after 5.5 days of differentiation. FIG. 1E depicts images of flow cytometric analyses of GFP⁺/cTnT⁺ and RFP⁺/cTnT⁺ cells at day 9 in cardiac spheroids.

FIG. 2A-2F are images depicting that PSC-derived FHF/SHF cells were similar to FHF/SHF cells in embryos. FIG. 2A depicts an image of RNA-seq analysis of differentially regulated genes between Hcn4-GFP⁺ and Tbx1-Cre, RFP⁺ CPCs in vivo and in vitro. The DESeq2 package identified 1,454 genes that were differentially regulated between Hcn4-GFP⁺ and Tbx1-cre, RFP⁺ CPCs in vivo and in vitro (Benjamini-Hochberg adjusted p-value<0.1). Upregulation in the GFP or RFP⁺ CPCs was determined using the directionality of fold change from DESeq2. 869 genes showed upregulation in the same CPC population both in vivo and in vitro. FIG. 2B depicts an image of Gene Ontology (GO) term analysis of 869 genes identified in FIG. 2A. Top ten biological processes enriched in the gene list (Bonferroni adjusted p-values<0.05) are shown. FIG. 2C depicts an image of RNA-seq heatmaps of CPCs both in vivo and in vitro using the 869 genes identified in FIG. 2A. Heatmaps show row-scaled regularized logarithmic transformation of counts as produced by the DESeq2 package. Hcn4-GFP⁺ and Tbx1-Cre, RFP⁺ CPCs cluster separately based on expression patterns of these genes both in vivo and in vitro. Select known FHF and SHF markers are labeled. FIG. 2D depicts bar graphs of qPCR analyses of selected genes involved in early CPC development of PSC-derived Hcn4-GFP CPCs and Tbx1-Cre, RFP isolated day 5.5. Data are mean±SEM; **p<0.01; ns, not significant (p>0.05). p values were determined using a paired Student's t test. FIG. 2E are images depicting immunohistochemistry analyses of cTnT, Pecam-1, Tny1 and aSMA at PSC-derived Hcn4-GFP CPCs and Tbx1-Cre, RFP CPCs isolated day 5.5 and analyzed day 9. White scale bars indicate 50 km. FIG. 2F depicts a line graph of cell counts of Hcn4-GFP⁺ CPCs and Tbx1-Cre, RFP⁺ CPCs isolated day 5.5. Data are mean±SEM; **p<0.01; ***p<0.001. p values were determined using a paired Student's t test.

FIG. 3A-3J depict images indicating that Bmp and Wnt activities regulated heart field specification in cardiac spheroids. FIG. 3A depicts a graph showing the Ingenuity Pathway Analysis of genes differentially expressed in Hcn4-GFP⁺ and Tbx1-Cre, RFP⁺ CPCs in vivo. The analysis was focused on pathways involved with “organism growth and development.” Data is shown as logarithm of Benjamini-Hochberg adjusted p-value, with threshold for significance of p-value<0.1. FIG. 3B depicts RNA-seq heatmaps of selected differentially regulated genes (Benjamini-Hochberg adjusted p-value<0.1) from Bmp signaling pathway and Wnt/β-catenin pathways. Data is shown as row-scaled regularized logarithmic transformation of counts as produced by the DESeq2 package. FIG. 3C is a graph depicting a vertical scatter plot showing the effect of increasing Bmp4 on formation of Hcn4-GFP⁺ CPCs (green trend line). FIG. 3D depicts a vertical scatter plot showing the effect of increasing Bmp4 on formation of Tbx1, RFP+ CPCs (red trend line). Both analyses (FIGS. 3C and 3D) were performed on GFP⁺/RFP⁺ percentages from flow cytometric analyses in FIG. 1D. FIG. 3E depicts a bar graph of the number of Hcn4-GFP⁺ CPCs and Tbx1-Cre, RFP⁺ CPCs after induction with increasing concentrations of Wnt3A. FIG. 3F depicts a bar graph of the number of Hcn4-GFP⁺ CPCs and Tbx1-cre, RFP⁺ CPCs after induction with Bmp4 (1.25 ng/ml) in combination with Wnt3A. FIG. 3G depicts a bar graph of qCR analyses of Tbx5, Hcn4, Tbx1 and Fgf10 after induction with Bmp4 (1.25 ng/ml) alone or in combination with Wnt3a (100 ng/ml), Wnt5A (100 ng/ml), Wnt11 (100 ng/ml) and IWP-2 (0.5 μM). FIG. 3H depicts representative FACS plots of Hcn4-GFP⁺ and Tbx1-cre, RFP⁺ CPCs at day 5.5. FIG. 3I depicts bar graphs of qPCR analyses of Tbx5, Hcn4, Tbx1 and Fgf10 after induction with Bmp4 (1.25 ng/ml) alone or in combination with Noggin (100 ng/ml) or dorsomorphin (100 nM), K2288 (100 nM) and DMH1 (100 nM). FIG. 3J depicts a bar graph of a Topflash assay after induction (88 h of differentiation) with Bmp4 (1.25 ng/ml) or Wnt3A (100 ng/ml) alone or in combination with Wnt3a, IWP-2, Noggin or Dorsomorphin. Data are mean±SEM; *p<0.05; **p<0.01. p values were determined using a paired Student's t test.

FIG. 4A-4G depict images showing that Cxcr4 marked SHF progenitors in mouse PSC-derived spheroids and in embryos. FIG. 4A depicts an image of an RNA-seq analysis of differentially expressed surface receptors between Hcn4-GFP⁺ and Tbx1-cre, RFP⁺ CPCs in vitro. 240 differentially expressed surface receptors were identified, of which the top 55 are shown here (all with Benjamini-Hochberg adjusted p-value<0.05). FIG. 4B depicts a graph of qPCR analysis of Cxcr4 expression in Hcn4-GFP⁺ and Tbx1-cre, RFP⁺ CPCs. Data are mean SEM; *p<0.05. p value was determined using a paired Student's t test. FIG. 4C depicts a representative flow histograms of Cxcr4 staining of Hcn4-GFP⁺ and Tbx1-cre, RFP⁺ CPCs compared to unstained GFP⁺/RFP⁺ CPCs. FIG. 4D depicts representative flow cytometric analysis and sorting strategy of Isl1-cre, RFP⁺ CPCs (left) and Cxcr4^{-/+} CPCs (right). FIG. 4E depicts a schematic of clonal cell-fate assay showing qPCR results from 2×24 single cell Cxcr4− and Cxcr4⁺ clones, sorted and plated at day 5.5 and analyzed 7 days later. Solid colors represent expression of the gene of interest (Ct value<30). Green: cTnT, Red: SM22, Blue: Pecam1, Yellow: FSP1/S100A4. FIG. 4F depicts images of Cxcr4−/+ CPCs isolated day 5.5, treated with EdU 24 h after isolation and stained for EdU and DAPI. Scale bars represent 50 μm, bar graph shows quantification of EdU+ CPCs, n=4. FIG. 4G depicts an image of a microarray analysis showing expression of cardiac genes in Isl1-cre, RFP⁺ Vs. Isl1-cre, RFP⁻ CPCs isolated day 5.5.

FIG. 5A-5G depict images showing that CXCR4 identifies SHF progenitors in human iPSC-derived spheroids. FIG. 5A is a schematic of the strategy used to generate and differentiate hiPSC-derived cardiac spheroids. FIG. 5B depicts a representative flow cytometric analyses showing the number of CXCR4⁻ and Cxcr4⁺ cells at day 5.5. (c) Representative flow cytometric analysis showing the number of human Isl1 cells in the sorted populations of CXCR4⁺ and CXCR4⁻ CPCs. FIG. 5D depicts a bar graph of qPCR analyses of CXCR4⁻ and CXCR4⁺ cells isolated at day 5.5. Data are mean±SEM; *p<0.05; **p<0.01; ns, not significant (p>0.05). p values were determined using a paired Student's t test. FIG. 5E depicts a bar graph of cell counts of Cxcr4⁻ and Cxcr4⁺ cells isolated day 5.5. Data are mean±SEM; **p<0.01. p values were determined using a paired Student's t test. FIG. 5F depicts a bar graph of qPCR analysis of Tnnt2, aSMA (smooth muscle cell marker), Fsp1 (fibroblast marker) and PECAM (endothelial cell marker) in cells derived from Cxcr4⁻ and Cxcr4⁺ CPCs isolated at day 5.5, re-plated as monolayers and isolated at day 12. Data are mean±SEM; *p<0.05; **p<0.01; ns, not significant (p>0.05). p values were determined using a paired Student's t test. FIG. 5G depicts representative images of human cardiomyocytes from Cxcr4⁻ and Cxcr4⁺ derived cardiomyocytes at day 12 (Above) and representative flow cytometric analyses of cTnT⁺ cardiomyocytes at day 12 (Below).

FIG. 6A-6O depict images showing validation of FHF/SHF markers, ESC derivation, optimization of cardiac differentiation in vitro. FIG. 6A is an illustration of FHF and SHF localization at cardiac crescent stage (E7.25-7.75) FIG. 6B is an image of Tbx1 (red) and Tbx5 (green) wholemount staining at E7.5. FIG. 6C is an image depicting Tbx1-cre linage trace analysis (red) and Tbx5 (green) staining in transverse E9.0 section. FIG. 6D is an image depicting Hcn4-GFP at E7.5 (top) and E9.5 (bottom). FIG. 6E is an image depicting wholemount Isl1-cre linage trace analysis (red) and Nkx2.5-GFP (green) at E7.5. FIG. 6F is an image depicting Isl1-cre linage trace analysis (red) and Nkx2.5-GFP (green) staining in transverse E9.0 section of embryo in (FIG. 6E). FIG. 6G is an image depicting wholemount Isl1-cre linage trace analysis (red) E9.5. FIG. 6H is an image depicting Isl1-cre linage trace analysis (red) and cTnT (green) staining in transverse E9.0 section. FIG. 6I is an image depicting Tbx1-cre linage trace analysis (red) and Hcn4-GFP (green) staining in transverse E9.0 section from embryo in FIG. 1B. FIG. 6J is a graph depicting the correlation between GFP+/RFP+ double positive cells and total number of GRP+ and RFP+ cells (FIG. 6K) Section of a spheroid day 7. White arrows indicate double positive cells. FIG. 6L depicts an image of flow cytometric analyses of cTnT in cardiac spheroids at day 9 of differentiation. FIG. 6M depicts an image of a vertical scatter plot of cTnT+ percentages in response to overall increasing Bmp4 concentrations. Data are mean±SEM; *p<0.05, **p<0.01, ns., not significant; p values were determined using one-way ANOVA analysis compared to 1.25 ng/ml Bmp4. FIG. 6N depicts an image of a vertical scatter plot of cTnT+ percentages in response to overall increasing Activin A concentrations. FIG. 6O depicts a bar graph of the percentage of GFP+, cTnT+ and RFP+, cTnT+ cardiomyocytes in cardiac spheroids. Data are mean±SEM; No individual sample was different compared to each other (p>0.05). p values were determined using one-way ANOVA analysis. White scale bars indicate 50 μm.

FIG. 7A-7I depict images of RNA-sequencing analysis, Tbx1 lineage tracing, KEGG pathway analysis. FIG. 7A depicts an image of scatter plots showing in vitro vs. in vivo Hcn4-GFP+ cells (green) and Tbx1-Cre, RFP+ cells (red). FIG. 7B depicts images of Hcn4 and Tbx1 violin expression level plots of in vitro and in vivo Hcn4-GFP+ and Tbx1-Cre, RFP+ samples. FIG. 7C depicts an image of GO terms analysis 585 genes that showed different expression patterns compared between in vitro and in vivo. FIG. 7D depicts an image of Tbx1-lineage trace of postnatal day 0 heart and immunohistochemistry analysis of cTnT, Pecam1 and Thy1 in Tbx1-Cre, RFP+ structures. FIG. 7E depicts images of scatter plots showing GFP and RFP percentages at day 9 (left) and cTnT+, Myogeninl+ and WT1+ cell percentages in RFP+ cells. (f) KEGG pathway analysis of cell cycle genes. FIG. 7G depicts an image of the KEGG pathway analysis of P53 signaling (red genes are upregulated in Tbx1-Cre, RFP+ CPCs, green genes are upregulated in Hcn4-GFP+ CPCs). FIG. 7H depicts a graph showing percentages of cTnT+ cardiomyocytes in GFP+ and RFP+ cells isolated and transfected with siRNA against tbx5 at day 5.5. Cells were analyzed 3 days after transfection. FIG. 7I depicts a graph showing proliferation analysis of GFP+ and RFP+ cells 3 days after isolation and transfection with siRNA against tbx1 at day 5.5. Data are mean±SEM; n=3; *p<0.05. p values were determined using a paired Student's t test.

FIGS. 8A and 8B depict images showing Isl1 levels in GFP⁺ and RFP+ cells at E7.75 and E8.5. FIG. 8A depicts a bar graph showing Isl1 levels in GFP⁺ and RFP+ cells at E7.5. FIG. 8B depicts a bar graph showing the relative Isl1 levels at E8.5 in GFP⁺ and RFP+ cells.

FIGS. 9A and 9B depict images showing the effect of Activin A on FHF/SHF specification and gene profiling. FIG. 9A depicts images of vertical scatter plots of GFP⁺ and RFP+ percentages in response to overall Bmp4 and Activin A concentrations from FIG. 1D. FIG. 9B depicts graphs of qPCR of analyses of differentiating mouse ESCs. Green dashed box indicate exposure to Bmp4 and gastrulation stage.

FIG. 10A-10G depict images showing the analysis of Cxcr4-positive progenitors in vivo and in vitro. FIG. 10A depicts graphs showing the expression levels of Cxcr4 and Epha2 along with Tbx5, Tbx1, Isl1, Fgf10 Nkx2.5 and acTc1 in Mesp-cre, RFP+ and RFP− cells isolated from pharyngeal arches and in developing heart at E9.0. FIG. 10B depicts images of immunohistochemistry analysis of 2nd pharyngeal arch of Mesp1-cre, RFP linage trace analysis (red) and Cxcr4 (green). FIG. 10C depicts a graph of qPCR analyses of early heart field genes in isolated Isl1-cre, RFP+, Cxcr4−/+ cells at day 5.5. FIG. 10D depicts representative flow cytometric analyses of RFP−, Isl1-Cre, RFP+, Cxcr4− and Isl1-Cre, RFP+, Cxcr4⁺ cells isolated at day 5.5. FIG. 10E depicts a graph showing cardiac muscle α-actinin 1 (actc1) levels in Isl1-Cre, RFP+, Cxcr4−/Cxcr4+ derived cells isolated and transfected with siRNA against tbx5 at day 5.5. Cells were analyzed 3 days after transfection. FIG. 10F depicts a graph showing proliferation analysis of Isl1-Cre, RFP+, Cxcr4−/Cxcr4⁺ cells 24 h and 48 h after isolation and transfection with siRNA against tbx1 at day 5.5. FIG. 10G depicts representative flow cytometric analyses of Isl1-Cre, RFP; Cxcr4 and Epha2 cells at day 5.5, and qPCR analyses of Isl1-Cre, RFP+, Cxcr4+/− and Epha2+/− cells. All data are mean±SEM; *p<0.05; n=3; p values were determined using a paired Student's t test.

FIG. 11 depicts graphs showing qPCR analyses of cardiac lineage markers in hiPSCs and CXCR4+/− cells sorted at day 5.5 All data are mean±SEM; *p<0.05; **p<0.01; n=3; p values were determined using a paired Student's t test.

FIG. 12 is a representative schematic of the methods described herein.

### DETAILED DESCRIPTION

The invention is based, at least in part, on the identification of differentially regulated pathways specify first heart field (FHF) and second heart field (SHF) formation. Also provided, is the identification that the cell surface protein Cxcr4 distinguishes SHF formation. The disclosure provided herein can be leveraged to generate heart field-specific progenitors for PSC-based modeling of heart field/chamber-specific diseases.

Over the past few decades, major advances have been made in identifying the origins of cardiac cells from developing embryos. In particular, the discovery of the first heart field (FHF) and the second heart field (SHF) led us to understand how diverse lineages and structures of the heart arise during cardiogenesis. However, it remains unknown how the two heart fields are specified and segregated, a fundamental step toward understanding heart formation and developing pluripotent stem cell (PSC)-based therapeutic strategies. Here, 3-dimensional spheroids were generated with mouse PSCs that harbor green and red fluorescent protein (GFP and RFP) reporters under the control of the FHF marker Hcn4 and the SHF marker Tbx1, respectively. GFP+ cells and RFP+ cells appeared from two distinct areas of mesodermal cells and develop in a complementary fashion, similar to the in vivo process.

Consistently, these populations exhibited a high degree of similarities with FHF/SHF cells isolated from early embryos, determined by RNA-sequencing analysis. Through a series of bioinformatics approaches, it was found that Bmp and Wnt are among the most differentially regulated pathways in the two populations. Gain- and loss-of-function studies showed that Bmp signaling specifies FHF cells and SHF cells via the Bmp/Smad pathway and Wnt signaling, respectively. Additionally, it was further found that SHF cells can be distinguished and isolated by the surface protein Cxcr4. This study provides fundamental insights into understanding the specification of two cardiac origins, which can be leveraged to generate heart field-specific progenitors for PSC-based modeling of heart field/chamber-specific disease.

Recent advances in cardiac developmental biology have led us to learn how diverse lineages and different anatomical structures of the heart arise from the two sets of molecularly distinct cardiac progenitor cells (CPCs), referred to as the first and second heart field (FHF and SHF). However, it remains unclear how the FHF and SHF populations are specified from mesodermal progenitors and which factors and mechanisms regulate their induction.

In early developing embryos, proper interactions of morphogens, including bone morphogenetic proteins (Bmps), Wnts, fibroblast growth factors, activin/nodal, play critical roles in formation of the primitive streak, progression of gastrulation and mesodermal patterning in the anterior-posterior axis¹⁻⁵. While numerous loss- and gain-of-function studies have demonstrated the importance of these pathways in early heart development, their precise roles in heart field induction and allocation remain to be determined⁶. However, recent studies provided evidence that heart field progenitors are assigned to a specific developmental path from nascent mesoderm marked by basic-helix-loop-helix (bHLH) transcription factor Mesp1 during gastrulation⁷⁻⁸, suggesting that the specification occurs soon after formation of three germ layers. Several transcription factors are known to have essential roles for pre-cardiac mesoderm development^{9, 10}: the T-box transcription factor Eomesodermin and the bHLH Id family of genes promote formation of cardiovascular mesoderm by activating Mesp1 during gastrulation, which in turn regulates expression of genes belonging to the cardiac transcriptional machinery such as Hand2, Gata4, Nkx2.5, and Myocd¹¹⁻¹³. Retrospective lineage analyses revealed that Mesp1⁺ cells contribute to both heart fields¹⁴. The FHF, comprising the cardiac crescent, is identified by expression of Hcn4 and Tbx5^{15, 16} before giving rise to the left ventricle (LV) and part of the atria, whereas the SHF is marked by transient expression of Tbx1, Fgf8/10, Isl1, and Six2, and exclusively contributes to the outflow tract (OT), the right ventricle (RV) and part of the atria¹⁷⁻²². SHF cells are multipotent CPCs that can be fated to various cardiac cell types, such as cardiomyocytes, smooth muscle cells, endothelial cells, and fibroblast cells, while FHF cells mostly become cardiomyocytes^{8, 23}.

With the capability to differentiate into any type of body cell, pluripotent stem cells (PSCs) have emerged as a powerful tool to study development and disease²⁴⁻²⁶. Particularly, the development of human induced PSCs (iPSC) technology and robust cardiac differentiation protocols^{27, 28} has enabled the study of disease-causing cellular and molecular events that manifest in congenital heart defects (CHDs), the most common birth defect and birth-related deaths in humans. Both genetic and environmental influences have been implicated to cause disruption of the normal series of morphogenetic embryonic developmental events that affects the occurrence of heart abnormalities. CHDs are often restricted to regions of the heart arising from the FHF or SHF²⁹⁻³² and/or linked to mutations of genes that regulate development of the individual heart fields 16, 17, 19, 33, 34. This raises the question whether chamber-specific heart abnormalities originate from abnormal heart field development. Efforts in tissue engineering and 3 dimensional (3D) bioprinting are now focused on developing heart chamber-specific models and to generate chamber-specific heart tissue from hiPSCs to replace damaged heart muscle 35, 36. Yet, it remains unknown whether the distinct heart field populations can be generated in a PSC system.

Described herein, 3D spheroids (e.g., precardiac spheroids) were generated with PSCs that allows induction of FHF/SHF progenitors sharing a high degree of similarities with their in vivo counterparts. It was further demonstrated how Bmp and Wnt/β-catenin signaling control the specification of FHF and SHF progenitors in mouse and human PSCs, enabling selective induction of FHF or SHF cells. The heart field progenitors can be identified and isolated without transgene reporters by the cell surface protein Cxcr4 for PSC-based modeling of CHDs.

### Stem Cells

Embryonic stem cells (ES cells) are pluripotent stem cells derived from the inner cell mass of a blastocyst, an early-stage preimplantation embryo. Human embryos reach the blastocyst stage within 4-5 days post fertilization, at which time they consist of 50-150 cells. Isolating the embryoblast or inner cell mass (ICM) results in destruction of the blastocyst. Embryonic stem cells, derived from the blastocyst stage early mammalian embryos, are distinguished by their ability to differentiate into any cell type and by their ability to propagate. Embryonic stem cell properties include having a normal karyotype, maintaining high telomerase activity, and exhibiting remarkable long-term proliferative potential.

Embryonic stem cells of the inner cell mass are pluripotent, that is, they are able to differentiate to generate primitive ectoderm, which ultimately differentiates during gastrulation into all derivatives of the three primary germ layers: ectoderm, endoderm, and mesoderm. These include each of the more than 220 cell types in the adult body. Pluripotency distinguishes embryonic stem cells from adult stem cells found in adults. While embryonic stem cells can generate all cell types in the body, adult stem cells are multipotent and can produce only a limited number of cell types. Harnessing the pluripotent differentiation potential of embryonic stem cells in vitro provide a means of deriving cell or tissue types virtually to order. This would provide a radical new treatment approach to a wide variety of conditions where age, disease, or trauma has led to tissue damage or dysfunction.

Additionally, under defined conditions, embryonic stem cells are capable of propagating themselves indefinitely in an undifferentiated state and have the capacity when provided with the appropriate signals to differentiate, presumably via the formation of precursor cells, to almost all mature cell phenotypes. This allows embryonic stem cells to be employed as useful tools for both research and regenerative medicine, because they produce limitless numbers of themselves for continued research or clinical use. Because of their plasticity and potentially unlimited capacity for self-renewal, embryonic stem cell therapies are used for regenerative medicine and tissue replacement after injury or disease.

Diseases that could potentially be treated by pluripotent stem cells include a number of blood and immune-system related genetic diseases, cancers, and disorders, e.g., juvenile diabetes, Parkinson's, blindness, and spinal cord injuries. There is a technical problem of graft-versus-host disease associated with allogeneic stem cell transplantation. However, the problems associated with histocompatibility may be solved using autologous donor adult stem cells or therapeutic cloning. The therapeutic cloning performed by a method called somatic cell nuclear transfer (SCNT) may be advantageous against mitochondrial DNA (mtDNA) mutated diseases.

Induced Pluripotent Stem Cells (iPSCs)

Induced pluripotent stem cells, commonly abbreviated as iPS cells or iPSCs are a type of pluripotent stem cell artificially derived from a non-pluripotent cell, typically an adult somatic cell, by inducing a “forced” expression of certain genes and transcription factors. These transcription factors play a key role in determining the state of these cells and also highlight the fact that these somatic cells do preserve the same genetic information as early embryonic cells. The ability to induce cells into a pluripotent state was initially pioneered using mouse fibroblasts and four transcription factors, Oct4, Sox2, Klf4 and c-Myc—called reprogramming. The successful induction of human iPSCs derived from human dermal fibroblasts has been performed using methods similar to those used for the induction of mouse cells. These induced cells exhibit similar traits to those of embryonic stem cells (ESCs) but do not require the use of embryos. Some of the similarities between ESCs and iPSCs include pluripotency, morphology, self-renewal ability, a trait that implies that they can divide and replicate indefinitely, and gene expression. Cardiac progenitor cells (or CPCs) are one type of pluripotent stem cell.

Current research focuses on differentiating ES into a variety of cell types for eventual use as cell replacement therapies (CRTs). Some of the cell types that have or are being developed include cardiomyocytes (CM), neurons, hepatocytes, bone marrow cells, islet cells and endothelial cells. Besides becoming an important alternative to organ transplants, ES are also being used in field of toxicology and as cellular screens to uncover new chemical entities (NCEs) that can be developed as small molecule drugs. Studies have shown that cardiomyocytes derived from ES are validated in in vitro models to test drug responses and predict toxicity profiles.

Adult stem cells, also called somatic stem cells, are stem cells which maintain and repair the tissue in which they are found. They can be found in children, as well as adults. Pluripotent adult stem cells are rare and generally small in number, but they can be found in umbilical cord blood and other tissues. Bone marrow is a rich source of adult stem cells, which have been used in treating several conditions including spinal cord injury, liver cirrhosis, chronic limb ischemia, and endstage heart failure. The quantity of bone marrow stem cells declines with age and is greater in males than females during reproductive years. Much adult stem cell research has aimed to characterize their potency and self-renewal capabilities. DNA damage accumulates with age in both stem cells and the cells that comprise the stem cell environment. This accumulation is considered to be responsible, at least in part, for increasing stem cell dysfunction with aging (see DNA damage theory of aging).

In adult organisms, stem cells and progenitor cells act as a repair system for the body, replenishing adult tissues. In a developing embryo, stem cells can differentiate into all the specialized cells—ectoderm, endoderm and mesoderm (see induced pluripotent stem cells)—but also maintain the normal turnover of regenerative organs, such as blood, skin, or intestinal tissues. There are three known accessible sources of autologous adult stem cells in humans: 1. Bone marrow, which requires extraction by harvesting, that is, drilling into bone (typically the femur or iliac crest). 2. Adipose tissue (lipid cells), which requires extraction by liposuction. 3. Blood, which requires extraction through apheresis, wherein blood is drawn from the donor (similar to a blood donation), and passed through a machine that extracts the stem cells and returns other portions of the blood to the donor. Stem cells can also be taken from umbilical cord blood just after birth. Of all stem cell types, autologous harvesting involves the least risk. By definition, autologous cells are obtained from one's own body.

Most adult stem cells are lineage-restricted (multipotent) and are generally referred to by their tissue origin (mesenchymal stem cell, adipose-derived stem cell, endothelial stem cell, dental pulp stem cell, etc.). Adult stem cell treatments have been successfully used for many years to treat leukemia and related bone/blood cancers through bone marrow transplants. Adult stem cells are also used in veterinary medicine to treat tendon and ligament injuries in horses. In instances where adult stem cells are obtained from the intended recipient (an autograft), the risk of rejection is essentially non-existent.

Examples of the genes important for differentiation into mesoderm include, but are not limited to, IGF2, GATA6, GATA4, SNAI2, MESP1, T, EOMES, SOX17, BMP4, CDX2, MESP2, and SNAIL.

### Cardiac Progenitor Cells (CPCs)

Heart development involves an early assignment of two distinct chamber-specific cell populations, called cardiac progenitor cells (CPCs), which generate the first and the second heart field and subsequently serve as building blocks of the left and right ventricular heart chamber, respectively. Consequently, abnormal CPC development is closely associated with the etiology of congenital heart disease—the leading cause of birth defect-related deaths in humans. Due to the embryonic onset and complex nature, congenital heart disease is particularly difficult to study and currently no model systems exist that allow the study of the cellular and molecular events leading to congenital heart abnormalities.

In aspects, provided herein are methods to identify and isolate first and the second heart field populations from pluripotent stem cells using an advanced fluorescent-based 3D in vitro culture system.

In further aspects, the expression of specific surface proteins was identified that distinguished these populations and which can be used to isolate these specific cell populations for disease modeling, drug discovery studies and potentially cell-based therapeutics.

In aspects, methods for producing, identifying and isolating first heart field progenitor cells and second heart field cells from pluripotent stem cells are provided. The method comprising. In embodiments, the method comprises activating a BMP signaling pathway in a 3-dimensional cluster of cells, referred to as embryoid bodies for at least a portion of the time when BMP signaling is activated. Furthermore, the method comprises using a fluorescence-based reporter system (combination of knock-in and the cre-lox system), that discloses how first and the second heart field progenitors are specified in pluripotent stem cells.

In aspects, the expression of specific surface proteins (Cxcr4 and EphA2) on second heart field progenitor cells, which can be used to identify and isolate these cells without the use of genetic labeling from human induced pluripotent cells was identified.

### Cardiomyocytes

Cardiac muscle cells or cardiomyocytes (also known as myocardiocytes or cardiac myocytes) are the muscle cells (myocytes) that make up the cardiac muscle. Each myocardial cell contains myofibrils, which are specialized organelles consisting of long chains of sarcomeres, the fundamental contractile units of muscle cells. Cardiomyocytes show striations similar to those on skeletal muscle cells, but unlike multinucleated skeletal cells, they contain only one nucleus. Cardiomyocytes have a high mitochondrial density, which allows them to produce adenosine triphosphate (ATP) quickly, making them highly resistant to fatigue.

There are two types of cells within the heart: the cardiomyocytes and the cardiac pacemaker cells. Cardiomyocytes make up the atria (the chambers in which blood enters the heart) and the ventricles (the chambers where blood is collected and pumped out of the heart). These cells must be able to shorten and lengthen their fibers and the fibers must be flexible enough to stretch. These functions are critical to the proper form during the beating of the heart.

Cardiac pacemaker cells carry the impulses that are responsible for the beating of the heart. They are distributed throughout the heart and are responsible for several functions. First, they are responsible for being able to spontaneously generate and send out electrical impulses. They also must be able to receive and respond to electrical impulses from the brain. Lastly, they must be able to transfer electrical impulses from cell to cell.

All of these cells are connected by cellular bridges. Porous junctions called intercalated discs form junctions between the cells. They permit sodium, potassium and calcium to easily diffuse from cell to cell. This makes it easier for depolarization and repolarization in the myocardium. Because of these junctions and bridges the heart muscle is able to act as a single coordinated unit.

Cardiac action potential consists of two cycles, a rest phase and an active phase. These two phases are commonly understood as systole and diastole. The rest phase is considered polarized. The resting potential during this phase of the beat separates the ions such as sodium, potassium and calcium. Myocardial cells possess the property of automaticity or spontaneous depolarization. This is the direct result of a membrane which allows sodium ions to slowly enter the cell until the threshold is reached for depolarization. Calcium ions follow and extend the depolarization even further. Once calcium stops moving inward, potassium ions move out slowly to produce repolarization. The very slow repolarization of the CMC membrane is responsible for the long refractory period.

Myocardial infarction, commonly known as a heart attack, occurs when the heart's supplementary blood vessels are obstructed by an unstable build-up of white blood cells, cholesterol, and fat. With no blood flow, the cells die, causing whole portions of cardiac tissue to die. Once these tissues are lost, they cannot be replaced, thus causing permanent damage.

Humans are born with a set number of heart muscle cells, or cardiomyocytes, which increase in size as our heart grows larger during childhood development. Recent evidence suggests that cardiomyocytes are actually slowly turned over as we age, but that less than 50% of the cardiomyocytes we are born with are replaced during a normal life span. The growth of individual cardiomyocytes not only occurs during normal heart development, it also occurs in response to extensive exercise (athletic heart syndrome), heart disease, or heart muscle injury such as after a myocardial infarction. A healthy adult cardiomyocyte has a cylindrical shape that is approximately 100 μm long and 10-25 μm in diameter. Cardiomyocyte hypertrophy occurs through sarcomerogenesis, the creation of new sarcomere units in the cell. During heart volume overload, cardiomyocytes grow through eccentric hypertrophy. The cardiomyocytes extend lengthwise but have the same diameter, resulting in ventricular dilation. During heart pressure overload, cardiomyocytes grow through concentric hypertrophy. The cardiomyocytes grow larger in diameter but have the same length, resulting in heart wall thickening.

### Heart Disease

Cardiovascular disease (CVD) is a class of diseases that involve the heart or blood vessels. Cardiovascular disease includes coronary artery diseases (CAD) such as angina and myocardial infarction (commonly known as a heart attack). Other CVDs are stroke, hypertensive heart disease, rheumatic heart disease, cardiomyopathy, atrial fibrillation, congenital heart disease, endocarditis, aortic aneurysms, peripheral artery disease and venous thrombosis.

A number of methods exist for diagnosing heart disease. Screening ECGs (Electrocardiogram—either at rest or with exercise) are one way to detect heart disease. Additionally echocardiography, myocardial perfusion imaging, and cardiac stress testing is not recommended in those at low risk who do not have symptoms. Some biomarkers may add to conventional cardiovascular risk factors in predicting the risk of future cardiovascular disease.

The underlying mechanisms vary depending on the disease in question. Coronary artery disease, stroke, and peripheral artery disease involve atherosclerosis. This may be caused by high blood pressure, smoking, diabetes, lack of exercise, obesity, high blood cholesterol, poor diet, and excessive alcohol consumption, among others. High blood pressure results in 13% of CVD deaths, while tobacco results in 9%, diabetes 6%, lack of exercise 6% and obesity 5%. Rheumatic heart disease may follow untreated strep throat.

There are many cardiovascular diseases involving the blood vessels. They are known as vascular diseases: Coronary artery disease (also known as coronary heart disease and ischemic heart disease), Peripheral arterial disease—disease of blood vessels that supply blood to the arms and legs, Cerebrovascular disease—disease of blood vessels that supply blood to the brain (includes stroke), Renal artery stenosis and Aortic aneurysm. There are also many cardiovascular diseases that involve the heart including but not limited to: Cardiomyopathy—diseases of cardiac muscle, Hypertensive heart disease—diseases of the heart secondary to high blood pressure or hypertension, Heart failure, Pulmonary heart disease—a failure at the right side of the heart with respiratory system involvement, Cardiac dysrhythmias—abnormalities of heart rhythm, Inflammatory heart disease, Endocarditis—inflammation of the inner layer of the heart, the endocardium. (The structures most commonly involved are the heart valves.) Inflammatory cardiomegaly, Myocarditis—inflammation of the myocardium, the muscular part of the heart, Valvular heart disease, Congenital heart disease—heart structure malformations existing at birth, Rheumatic heart disease—heart muscles and valves damage due to rheumatic fever caused by Streptococcus pyogenes a group A streptococcal infection.

Dilated cardiomyopathy (DCM) is one of the cardiomyopathies, a group of diseases that primarily affect the myocardium. In DCM a portion of the myocardium is dilated, often without any obvious cause. Left or right ventricular systolic pump function of the heart is impaired, leading to progressive cardiac enlargement and hypertrophy, a process called remodeling. Although in many cases no etiology is apparent, dilated cardiomyopathy can result from a variety of toxic, metabolic, or infectious agents. About 25-35% of patients have familial forms of the disease, with most mutations affecting genes encoding cytoskeletal proteins, while some affect other proteins involved in contraction. The disease is genetically heterogeneous, but the most common form of its transmission is an autosomal dominant pattern. Cytoskeletal proteins involved in DCM include cardiac troponin T (TNNT2), α-cardiac actin, desmin, and the nuclear lamins A and C, and various other contractile proteins.

Hypertrophic cardiomyopathy (HCM), is a condition in which sarcomeres replicate causing heart muscle cells to increase in size, which results in the thickening of the heart muscle. In addition, the normal alignment of muscle cells is disrupted, a phenomenon known as myocardial disarray. HCM also causes disruptions of the electrical functions of the heart. HCM is most commonly due to a mutation in one of 9 sarcomeric genes that results in a mutated protein in the sarcomere. Myosin heavy chain mutations are associated with development of familial hypertrophic cardiomyopathy. Hypertrophic cardiomyopathy is usually inherited as an autosomal dominant trait, which mutations reported in cardiac troponin T (TNNT2); myosin heavy chain (MYH7); tropomyosin 1 (TPM1); myosin binding protein C (MYBPC3); 5′-AMP-activated protein kinase subunit gamma-2 (PRKAG2); troponin I type 3 (TNNI3); titin (UN); myosin, light chain 2 (MYL2); actin, alpha cardiac muscle 1 (ACTC1); and cardiac LIM protein (CSRP3). An insertion/deletion polymorphism in the gene encoding for angiotensin converting enzyme (ACE) alters the clinical phenotype of the disease. The D/D (deletion/deletion) genotype of ACE is associated with more marked hypertrophy of the left ventricle and may be associated with higher risk of adverse outcomes.

Anthracycline-induced cardiotoxicity (and resistance to anthracycline-induced toxicity). Anthracyclines such as doxorubicin are frontline chemotherapeutic agents that are used to treat leukemias, Hodgkin's lymphoma, and solid tumors of the breast, bladder, stomach, lung, ovaries, thyroid, and muscle, among other organs. The primary side effect of anthracyclines is cardiotoxicity, which results in severe heart failure for many of the recipients receiving regimens utilizing this chemotherapeutic agent.

Arrhythmogenic right ventricular dysplasia (ARVD). ARVD is an autosomal dominant disease of cardiac desmosomes that results in arrhythmia of the right ventricle and sudden cardiac death. It is second only to hypertrophic cardiomyopathy as a leading cause for sudden cardiac death in the young.

Left Ventricular Non-Compaction (LVNC, aka non-compaction cardiomyopathy). LVNC is a hereditary cardiac disease which results from impaired development of the myocardium (heart muscle) during embryogenesis. Patients with mutations causing LVNC develop heart failure and abnormal cardiac electrophysiology early in life.

Double Inlet Left Ventricle (DILV). DILV is a congenital heart defect in which both the left and right atria feed into the left ventricle. As a result, children born with this defect only have one functional ventricular chamber, and trouble pumping oxygenated blood into the general circulation.

Long QT (Type-1) Syndrome (LOT-1, KCNQ1 mutation). Long QT syndrome (LOT) is a hereditary arrhythmic disease in which the QT phase of the electrocardiogram is prolonged, resulting in increased susceptibility for arrhythmia and sudden cardiac death. There are 13 known genes associated with LQT.

The most common congenital heart defects include ventricular septal defects, atrial septal defects, and patent ductus arteriosus. Left-to-right ventricular septal defects and patent ductus arteriosus typical result in the left side of the heart having to work harder because some of the blood it pumps will recirculate through the lungs instead of circulating throughout the body. Atrial septal defects typically result in blood being shunted from the left atrium to the right, thus overloading the right side of the heart. These conditions have significant consequences if left untreated including hypertension, increased pulmonary arterial pressure, strain on the heart muscle, and ultimately heart failure.

There are several risk factors for heart diseases: age, gender, tobacco use, physical inactivity, excessive alcohol consumption, unhealthy diet, obesity, family history of cardiovascular disease, raised blood pressure (hypertension), raised blood sugar (diabetes mellitus), raised blood cholesterol (hyperlipidemia), psychosocial factors, poverty and low educational status, and air pollution. While the individual contribution of each risk factor varies between different communities or ethnic groups the overall contribution of these risk factors is very consistent. Some of these risk factors, such as age, gender or family history, are immutable; however, many important cardiovascular risk factors are modifiable by lifestyle change, social change, drug treatment and prevention of hypertension, hyperlipidemia, and diabetes.

### Cardiomyopathy

Cardiomyopathy (literally “heart muscle disease”) is the measurable deterioration for any reason of the ability of the myocardium (the heart muscle) to contract, usually leading to heart failure. Common symptoms include dyspnea (breathlessness) and peripheral edema (swelling of the legs). Those with cardiomyopathy are often at risk of dangerous forms of irregular heart rate and sudden cardiac death. The most common form of cardiomyopathy is dilated cardiomyopathy. Although the term “cardiomyopathy” could theoretically apply to almost any disease affecting the heart, it is usually reserved for “severe myocardial disease leading to heart failure”. Cardiomyopathy and myocarditis resulted in 443,000 deaths in 2013, up from 294,000 in 1990.

Cardiomyopathies are either confined to the heart or are part of a generalized disorder, both often leading to death or progressive heart failure. Other diseases that cause heart muscle dysfunction are excluded, such as coronary artery disease, hypertension, or abnormalities of the heart valves.

Earlier, simpler, categories such as intrinsic, (defined as weakness of the heart muscle without an identifiable external cause), and extrinsic, (where the primary pathology arose outside the myocardium itself), became more difficult to sustain. For example, as more external causes were recognized, the intrinsic category became smaller. Alcoholism, for example, has been identified as a cause of dilated cardiomyopathy, as has drug toxicity, and certain infections (including Hepatitis C). On the other hand, molecular biology and genetics have given rise to the recognition of various genetic causes, increasing the intrinsic category. For example, mutations in the cardiac desmosomal genes as well as in the DES gene may cause arrhythmogenic right ventricular cardiomyopathy (ARVC).

At the same time, a more clinical categorization of cardiomyopathy as ‘hypertrophied’, ‘dilated’, or ‘restrictive’, became difficult to maintain when it became apparent that some of the conditions could fulfill more than one of those three categories at any particular stage of their development. The current American Heart Association definition divides cardiomyopathies into primary, which affect the heart alone, and secondary, which are the result of illness affecting other parts of the body. These categories are further broken down into subgroups which incorporate new genetic and molecular biology knowledge.

Cardiomyopathies can be classified using different criteria. Structural categories of cardiomyopathy include but are not limited to: Primary/intrinsic cardiomyopathies, Genetic Hypertrophic cardiomyopathy, Arrhythmogenic right ventricular cardiomyopathy (ARVC), LV non-compaction, Ion Channelopathies, Dilated cardiomyopathy (DCM), Restrictive cardiomyopathy (RCM), Aquired Cardiommyopathy, Stress Cardiomyopathy, Myocarditis, and Ischemic cardiomyopathy. Secondary/extrinsic cardiomyopathies include but are not limited to: Metabolic/storage disease, Fabry's disease, hemochromatosis, Endomyocardial fibrosis, Hypereosinophilic syndrome, diabetes mellitus, hyperthyroidism, acromegaly, Noonan syndrome, muscular dystrophy, Friedreich's ataxia, and Obesity-associated cardiomyopathy.

Symptoms may include shortness of breath after physical exertion, fatigue, and swelling of the feet, legs, or abdomen. Additionally, arrhythmias and chest pain may be present. The pathophysiology of cardiomyopathies is better understood at the cellular level with advances in molecular techniques. Mutant proteins can disturb cardiac function in the contractile apparatus (or mechanosensitive complexes). Cardiomyocyte alterations and their persistent responses at the cellular level cause changes that are correlated with sudden cardiac death and other cardiac problems. A number of methods exist for detecting the presence of cardiomyopathy in a patient. Among the diagnostic procedures done to determine a cardiomyopathy are: Physical exam, Family history, Blood test, EKG, Echocardiogram, Stress test, and Genetic testing.

Treatment may include suggestion of lifestyle changes to better manage the condition. Treatment depends on the type of cardiomyopathy and condition of disease, but may include medication (conservative treatment) or iatrogenic/implanted pacemakers for slow heart rates, defibrillators for those prone to fatal heart rhythms, ventricular assist devices (VADs) for severe heart failure, or ablation for recurring dysrhythmias that cannot be eliminated by medication or mechanical cardioversion. The goal of treatment is often symptom relief, and some patients may eventually require a heart transplant.

### Cardiac Morphogenesis (Formation of the Embryonic Heart)

Formation of the embryonic heart has been characterized (e.g., Kelly, R. “Heart Fields and Cardiac Morphogenesis” Cold Spring Harb Perspect Med v.4(10) 2014; incorporated herein by reference in its entirety). Cells that give rise to the early heart tube are specified and differentiate in lateral anterior splanchnic mesoderm as a result of combinatorial signals from surrounding tissues. Cranial mesoderm is derived from progenitor cells that activate the bHLH transcription factor MESP1 in the primitive streak, under control of the T-box factor Eomesodermin (Saga et al. 2000; Costello et al. 2011). The pattern of inductive signals from adjacent endoderm and overlying ectoderm together with inhibitory signals from the embryonic midline and posterior region of the embryo refine the sites in which the cardiomyogenic transcriptional program is first activated (Marvin et al. 2001; Harvey 2002; Lopez-Sanchez and Garcia-Martinez 2011). These signals, including bone morphogenetic protein (BMP), fibroblast growth factor (FGF), and WNT signals, in addition to short range signaling including fibronectin mediated cascades, result in the activation of key upstream transcriptional regulators of the cardiac phenotype including genes encoding the transcription factors NKX2-5, GATA4, and TBX5, and chromatin remodeling protein SMARCD3 (BAF60c) (Lopez-Sanchez and Garcia-Martinez 2011; Cheng et al. 2013).

The embryonic heart is comprised of cardiomyocytes derived from the cardiac crescent and linear heart as well as those derived from second heart field progenitor cells in pharyngeal mesoderm. Retrospective lineage analysis and genetic tracing using Cre recombinase support a two lineage model of heart development corresponding to the contributions of the first and second heart fields (Cai et al. 2003; Meilhac et al. 2004). Furthermore, a population of late differentiating cardiomyocytes has been found to add to the poles of the frog and fish heart suggesting that this mechanism for heart tube elongation is evolutionarily conserved across vertebrate species (de Pater et al. 2009; Gessert and Kuhl 2009; Hami et al. 2011; Lazic and Scott 2011; Zhou et al. 2011).

### C-X-C Chemokine Receptor Type 4 (CXCR-4)

Cxcr4, also known as fusin or CD184 (cluster of differentiation 184) is a protein that in humans is encoded by the CXCR4 gene. CXCR-4 is an alpha-chemokine receptor specific for stromal-derived-factor-1 (SDF-1 also called CXCL12), a molecule endowed with potent chemotactic activity for lymphocytes. CXCR4 is one of several chemokine receptors that HIV can use to infect CD4+ T cells. HIV isolates that use CXCR4 are traditionally known as T-cell tropic isolates. Typically, these viruses are found late in infection. CXCR4 is upregulated during the implantation window in natural and hormone replacement therapy cycles in the endometrium, producing, in presence of a human blastocyst, a surface polarization of the CXCR4 receptors suggesting that this receptor is implicated in the adhesion phase of human implantation.

CXCR4's ligand SDF-1 is known to be important in hematopoietic stem cell homing to the bone marrow and in hematopoietic stem cell quiescence. It has been also shown that CXCR4 signaling regulates the expression of CD20 on B cells. Until recently, SDF-1 and CXCR4 were believed to be a relatively monogamous ligand-receptor pair (other chemokines are promiscuous, tending to use several different chemokine receptors). Recent evidence demonstrates ubiquitin is also a natural ligand of CXCR4. Ubiquitin is a small (76-amino acid) protein highly conserved among eukaryotic cells. It is best known for its intracellular role in targeting ubiquitylated proteins for degradation via the ubiquitin proteasome system. Evidence in numerous animal models suggests ubiquitin is anti-inflammatory immune modulator and endogenous opponent of proinflammatory damage associated molecular pattern molecules. It is speculated this interaction may be through CXCR4 mediated signaling pathways. MIF is an additional ligand of CXCR4.

CXCR4 is present in newly generated neurons during embryogenesis and adult life where it plays a role in neuronal guidance. The levels of the receptor decrease as neurons mature. CXCR4 mutant mice have aberrant neuronal distribution. This has been implicated in disorders such as epilepsy.

### Cxcr4 Clinical Significance

Drugs that block the CXCR4 receptor appear to be capable of “mobilizing” hematopoietic stem cells into the bloodstream as peripheral blood stem cells. Peripheral blood stem cell mobilization is very important in hematopoietic stem cell transplantation (as a recent alternative to transplantation of surgically harvested bone marrow) and is currently performed using drugs such as G-CSF. G-CSF is a growth factor for neutrophils (a common type of white blood cells), and may act by increasing the activity of neutrophil-derived proteases such as neutrophil elastase in the bone marrow leading to proteolytic degradation of SDF-1. Plerixafor (AMD3100) is a drug, approved for routine clinical use, which directly blocks the CXCR4 receptor. It is an efficient inducer of hematopoietic stem cell mobilization in animal and human studies. In a small human clinical trial to evaluate the safety and efficacy of fucoidan ingestion (brown seaweed extract), 3 g daily of 75% w/w oral fucoidan for 12 days increased the proportion of CD34+ CXCR4+ from 45 to 90% and the serum SDF-1 levels, which could be useful in CD34+ cells homing/mobilization via SDF-1/CXCR4 axis.

While CXCR4's expression is low or absent in many healthy tissues, it was demonstrated to be expressed in over 23 types of cancer, including breast cancer, ovarian cancer, melanoma, and prostate cancer. Expression of this receptor in cancer cells has been linked to metastasis to tissues containing a high concentration of CXCL12, such as lungs, liver and bone marrow. However, in breast cancer where SDF1/CXCL12 is also expressed by the cancer cells themselves along with CXCR4, CXCL12 expression is positively correlated with disease free (metastasis free) survival. CXCL12 (over-)expressing cancers might not sense the CXCL12 gradient released from the metastasis target tissues since the receptor, CXCR4, is saturated with the ligand produced in an autocrine manner. Another explanation of this observation is provided by a study that shows the ability of CXCL12 (and CCL2) producing tumors to entrain neutrophils that inhibit seeding of tumor cells in the lung.

An amino acid sequence for human Cxcr4 is publically available in the GenBank database accession number NP_003458.1 (SEQ ID NO: 1) and is as follows:

| | |
|---|---|
| 1 | megisiytsd nyteemgsgd ydsmkepcfr eenanfnkif lptiysiifl tgivgnglvi |
| 61 | lvmgyqkklr smtdkyrlhl svadllfvit lpfwavdava nwyfgnflck avhviytvnl |
| 121 | yssvlilafi sldrylaivh atnsgrprkl laekvvyvgv wipallltip dfifanvsea |
| 181 | ddryicdrfy pndlwvvvfq fqhimvglil pgivilscyc iiisklshsk ghqkrkalkt |
| 241 | tvililaffa cwlpyyigis idsfilleii kqgcefentv hkwisiteal affhcclnpi |
| 301 | lyaflgakfk tsaqhaltsv srgsslkils kgkrgghssv stesesssfh ss |

A nucleotide sequence that encodes human Cxcr4 is publically available in the GenBank database accession number NM_003467 (SEQ ID NO: 2) and is as follows (the start and stop codon are bold and underlined).

| | |
|---|---|
| 1 | aacttcagtt tgttggctgc ggcagcaggt agcaaagtga cgccgagggc ctgagtgctc |
| 61 | cagtagccac cgcatctgga gaaccagcgg ttaccatgga ggggatcagt atatacactt |
| 121 | cagataacta caccgaggaa atgggctcag gggactatga ctccatgaag gaaccctgtt |
| 181 | tccgtgaaga aaatgctaat ttcaataaaa tcttcctgcc caccatctac tccatcatct |
| 241 | tcttaactgg cattgtgggc aatggattgg tcatcctggt catgggttac cagaagaaac |
| 301 | tgagaagcat gacggacaag tacaggctgc acctgtcagt ggccgacctc ctctttgtca |
| 361 | tcacgcttcc cttctgggca gttgatgccg tggcaaactg gtactttggg aacttcctat |
| 421 | gcaaggcagt ccatgtcatc tacacagtca acctctacag cagtgtcctc atcctggcct |
| 481 | tcatcagtct ggaccgctac ctggccatcg tccacgccac caacagtcag aggccaagga |
| 541 | agctgttggc tgaaaaggtg gtctatgttg gcgtctggat ccctgccctc ctgctgacta |
| 601 | ttcccgactt catctttgcc aacgtcagtg aggcagatga cagatatatc tgtgaccgct |
| 661 | tctaccccaa tgacttgtgg gtggttgtgt tccagtttca gcacatcatg gttggcctta |
| 721 | tcctgcctgg tattgtcatc ctgtcctgct attgcattat catctccaag ctgtcacact |
| 781 | ccaagggcca ccagaagcgc aaggccctca agaccacagt catcctcatc ctggctttct |
| 841 | tcgcctgttg gctgccttac tacattggga tcagcatcga ctccttcatc ctcctggaaa |
| 901 | tcatcaagca agggtgtgag tttgagaaca ctgtgcacaa gtggatttcc atcaccgagg |
| 961 | ccctagcttt cttccactgt tgtctgaacc ccatcctcta tgctttcctt ggagccaaat |
| 1021 | ttaaaacctc tgcccagcac gcactcacct ctgtgagcag agggtccagc ctcaagatcc |
| 1081 | tctccaaagg aaagcgaggt ggacattcat ctgtttccac tgagtctgag tcttcaagtt |
| 1141 | ttcactccag ctaacacaga tgtaaaagac ttttttttat acgataaata actttttttt |
| 1201 | aagttacaca tttttcagat ataaaagact gaccaatatt gtacagtttt tattgcttgt |
| 1261 | tggatttttg tcttgtgttt ctttagtttt tgtgaagttt aattgactta tttatataaa |
| 1321 | ttttttttgt ttcatattga tgtgtgtcta ggcaggacct gtggccaagt tcttagttgc |
| 1381 | tgtatgtctc gtggtaggac tgtagaaaag ggaactgaac attccagagc gtgtagtgaa |
| 1441 | tcacgtaaag ctagaaatga tccccagctg tttatgcata gataatctct ccattcccgt |
| 1501 | ggaacgtttt tcctgttctt aagacgtgat tttgctgtag aagatggcac ttataaccaa |
| 1561 | agcccaaagt ggtatagaaa tgctggtttt tcagttttca ggagtgggtt gatttcagca |
| 1621 | cctacagtgt acagtcttgt attaagttgt taataaaagt acatgttaaa cttaaaaaaa |
| 1681 | aaaaaaaaaa a |

### EphA2 (Ephrin Type-A Receptor 2)

This gene belongs to the ephrin receptor subfamily of the protein-tyrosine kinase family. EPH and EPH-related receptors have been implicated in mediating developmental events, particularly in the nervous system. Receptors in the EPH subfamily typically have a single kinase domain and an extracellular region containing a Cys-rich domain and 2 fibronectin type III repeats. The ephrin receptors are divided into two groups based on the similarity of their extracellular domain sequences and their affinities for binding ephrin-A and ephrin-B ligands. This gene encodes a protein that binds ephrin-A ligands.

A protein sequence that encodes human EphA2 is publically available in the GenBank database accession number NP_001316019 (SEQ ID NO: 3) and is as follows:

| | |
|---|---|
| 1 | mgnimndmpi ymysvcnvms gdqdnwlrtn wvyrgeaeri fielkftvrd cnsfpggass |
| 61 | cketfnlyya esdldygtnf qkrlftkidt iapdeitvss dfearhvkln veersvgplt |
| 121 | rkgfylafqd igacvallsv rvyykkcpel lqglahfpet iagsdapsla tvagtcvdha |
| 181 | vvppggeepr mhcavdgewl vpiggcicqa gyekvedacq acspgffkfe asespclecp |
| 241 | ehtlpspega tsceceegff rapqdpasmp ctrppsaphy ltavgmgakv elrwtppqds |
| 301 | ggredivysv tceqcwpesg ecgpceasvr ysepphgltr tsvtvsdlep hmnytftvea |
| 361 | rngvsglvts rsfrtasysi ngteppkvrl egrsttslsv swsipppqqs rvwkyevtyr |
| 421 | kkgdsnsynv rrtegfsvtl ddlapdttyl vqvgaltgeg qgagskvhef qtlspegsgn |
| 481 | laviggvavg vvlllvlagv gffihrrrkn grargspedv yfskseqlkp lktyvdphty |
| 541 | edpnqavlkf tteihpscvt rqkvigagef gevykgmlkt ssgkkevpva iktlkagyte |
| 601 | kqrvdflgea gimgqfshhn iirlegvisk ykpmmiitey mengaldkfl rekdgefsvl |
| 661 | qlvgmlrgia agmkylanmn yvhrdlaarn ilvnsnlvck vsdfglsrvl eddpeatytt |
| 721 | sggkipirwt apeaisyrkf tsasdvwsfg ivmwevmtyg erpywelsnh evmkaindgf |
| 781 | rlptpmdcps aiyqlmmqcw ggerarrpkf adivsildkl irapdslktl adfdprvsir |
| 841 | lpstsgsegv pfrtvsewle sikmqqyteh fmaagytaie kvvqmtnddi krigvrlpgh |
| 901 | qkriaysllg lkdqvntvgi pi |

A nucleotide sequence that encodes human EphA2 is publically available in the GenBank database accession number NM_001329090.1 (SEQ ID NO: 4) and is as follows. The start and stop codons are bold and underlined.

| | |
|---|---|
| 1 | agggcatgaa tgaacaggag tcggttctca cccaacttcc attaaggact cggggcagga |
| 61 | ggggcagaag ttgcgcgcag gccggcgggc gggagcggac accgaggccg gcgtgcaggc |
| 121 | gtgcgggtgt gcgggagccg ggctcggggg gatcggaccg agagcgagaa gcgcggcatg |
| 181 | gagctccagg cagcccgcgc ctgcttcgcc ctgctgtggg gctgtgcgct ggccgcggcc |
| 241 | gcggcggcgc agggcaagga agtgggacct gatgcagaac atcatgaatg acatgccgat |
| 301 | ctacatgtac tccgtgtgca acgtgatgtc tggcgaccag gacaactggc tccgcaccaa |
| 361 | ctgggtgtac cgaggagagg ctgagcgtat cttcattgag ctcaagttta ctgtacgtga |
| 421 | ctgcaacagc ttccctggtg gcgccagctc ctgcaaggag actttcaacc tctactatgc |
| 481 | cgagtcggac ctggactacg gcaccaactt ccagaagcgc ctgttcacca agattgacac |
| 541 | cattgcgccc gatgagatca ccgtcagcag cgacttcgag gcacgccacg tgaagctgaa |
| 601 | cgtggaggag cgctccgtgg ggccgctcac ccgcaaaggc ttctacctgg ccttccagga |
| 661 | tatcggtgcc tgtgtggcgc tgctctccgt ccgtgtctac tacaagaagt gccccgagct |
| 721 | gctgcagggc ctggcccact tccctgagac catcgccggc tctgatgcac cttccctggc |
| 781 | cactgtggcc ggcacctgtg tggaccatgc cgtggtgcca ccggggggtg aagagccccg |
| 841 | tatgcactgt gcagtggatg gcgagtggct ggtgcccatt gggcagtgcc tgtgccaggc |
| 901 | aggctacgag aaggtggagg atgcctgcca ggcctgctcg cctggatttt ttaagtttga |
| 961 | ggcatctgag agcccctgct tggagtgccc tgagcacacg ctgccatccc ctgagggtgc |
| 1021 | cacctcctgc gagtgtgagg aaggcttctt ccgggcacct caggacccag cgtcgatgcc |
| 1081 | ttgcacacga cccccctccg ccccacacta cctcacagcc gtgggcatgg gtgccaaggt |
| 1141 | ggagctgcgc tggacgcccc ctcaggacag cgggggccgc gaggacattg tctacagcgt |
| 1201 | cacctgcgaa cagtgctggc ccgagtctgg ggaatgcggg ccgtgtgagg ccagtgtgcg |
| 1261 | ctactcggag cctcctcacg gactgacccg caccagtgtg acagtgagcg acctggagcc |
| 1321 | ccacatgaac tacaccttca ccgtggaggc ccgcaatggc gtctcaggcc tggtaaccag |
| 1381 | ccgcagcttc cgtactgcca gtgtcagcat caaccagaca gagcccccca aggtgaggct |
| 1441 | ggagggccgc agcaccacct cgcttagcgt ctcctggagc atccccccgc cgcagcagag |
| 1501 | ccgagtgtgg aagtacgagg tcacttaccg caagaaggga gactccaaca gctacaatgt |
| 1561 | gcgccgcacc gagggtttct ccgtgaccct ggacgacctg gccccagaca ccacctacct |
| 1621 | ggtccaggtg caggcactga cgcaggaggg ccagggggcc ggcagcaagg tgcacgaatt |
| 1681 | ccagacgctg tccccggagg gatctggcaa cttggcggtg attggcggcg tggctgtcgg |
| 1741 | tgtggtcctg cttctggtgc tggcaggagt tggcttcttt atccaccgca ggaggaagaa |
| 1801 | ccagcgtgcc cgccagtccc cggaggacgt ttacttctcc aagtcagaac aactgaagcc |
| 1861 | cctgaagaca tacgtggacc cccacacata tgaggacccc aaccaggctg tgttgaagtt |
| 1921 | cactaccgag atccatccat cctgtgtcac tcggcagaag gtgatcggag caggagagtt |
| 1981 | tggggaggtg tacaagggca tgctgaagac atcctcgggg aagaaggagg tgccggtggc |
| 2041 | catcaagacg ctgaaagccg gctacacaga gaagcagcga gtggacttcc tcggcgaggc |
| 2101 | cggcatcatg ggccagttca gccaccacaa catcatccgc ctagagggcg tcatctccaa |
| 2161 | atacaagccc atgatgatca tcactgagta catggagaat ggggccctgg acaagttcct |
| 2221 | tcgggagaag gatggcgagt tcagcgtgct gcagctggtg ggcatgctgc ggggcatcgc |
| 2281 | agctggcatg aagtacctgg ccaacatgaa ctatgtgcac cgtgacctgg ctgcccgcaa |
| 2341 | catcctcgtc aacagcaacc tggtctgcaa ggtgtctgac tttggcctgt cccgcgtgct |
| 2401 | ggaggacgac cccgaggcca cctacaccac cagtggcggc aagatcccca tccgctggac |
| 2461 | cgccccggag gccatttcct accggaagtt cacctctgcc agcgacgtgt ggagctttgg |
| 2521 | cattgtcatg tgggaggtga tgacctatgg cgagcggccc tactgggagt tgtccaacca |
| 2581 | cgaggtgatg aaagccatca atgatggctt ccggctcccc acacccatgg actgcccctc |
| 2641 | cgccatctac cagctcatga tgcagtgctg gcagcaggag cgtgcccgcc gccccaagtt |
| 2701 | cgctgacatc gtcagcatcc tggacaagct cattcgtgcc cctgactccc tcaagaccct |
| 2761 | ggctgacttt gacccccgcg tgtctatccg gctccccagc acgagcggct cggagggggt |
| 2821 | gcccttccgc acggtgtccg agtggctgga gtccatcaag atgcagcagt atacggagca |
| 2881 | cttcatggcg gccggctaca ctgccatcga gaaggtggtg cagatgacca acgacgacat |
| 2941 | caagaggatt ggggtgcggc tgcccggcca ccagaagcgc atcgcctaca gcctgctggg |
| 3001 | actcaaggac caggtgaaca ctgtggggat ccccatctga gcctcgacag ggcctggagc |
| 3061 | cccatcggcc aagaatactt gaagaaacag agtggcctcc ctgctgtgcc atgctgggcc |
| 3121 | actggggact ttatttattt ctagttcttt cctccccctg caacttccgc tgaggggtct |
| 3181 | cggatgacac cctggcctga actgaggaga tgaccaggga tgctgggctg ggccctcttt |
| 3241 | ccctgcgaga cgcacacagc tgagcactta gcaggcaccg ccacgtccca gcatccctgg |
| 3301 | agcaggagcc ccgccacagc cttcggacag acatatggga tattcccaag ccgaccttcc |
| 3361 | ctccgccttc tcccacatga ggccatctca ggagatggag ggcttggccc agcgccaagt |
| 3421 | aaacagggta cctcaagccc catttcctca cactaagagg gcagactgtg aacttgactg |
| 3481 | ggtgagaccc aaagcggtcc ctgtccctct agtgccttct ttagaccctc gggccccatc |
| 3541 | ctcatccctg actggccaaa cccttgcttt cctgggcctt tgcaagatgc ttggttgtgt |
| 3601 | tgaggttttt aaatatatat tttgtacttt gtggagagaa tgtgtgtgtg tggcaggggg |
| 3661 | ccccgccagg gctggggaca gagggtgtca aacattcgtg agctggggac tcagggaccg |
| 3721 | gtgctgcagg agtgtcctgc ccatgcccca gtcggcccca tctctcatcc ttttggataa |
| 3781 | gtttctattc tgtcagtgtt aaagattttg ttttgttgga catttttttc gaatcttaat |
| 3841 | ttattatttt ttttatattt attgttagaa aatgacttat ttctgctctg gaataaagtt |
| 3901 | gcagatgatt caaaccgaaa aaaa |

### Wnt Signaling

The Wnt signaling pathways are a group of signal transduction pathways made of proteins that pass signals into a cell through cell surface receptors. Three Wnt signaling pathways have been characterized: the canonical Wnt pathway, the noncanonical planar cell polarity pathway, and the noncanonical Wnt/calcium pathway. All three pathways are activated by binding a Wnt-protein ligand to a Frizzled family receptor, which passes the biological signal to the Disheveled protein inside the cell. The canonical Wnt pathway leads to regulation of gene transcription, and is thought to be negatively regulated in part by the SPATS1 gene. The noncanonical planar cell polarity pathway regulates the cytoskeleton that is responsible for the shape of the cell. The noncanonical Wnt/calcium pathway regulates calcium inside the cell. Wnt signaling pathways use either nearby cell-cell communication (paracrine) or same-cell communication (autocrine). They are highly evolutionarily conserved in animals, which means they are similar across animal species from fruit flies to humans.

Wnt signaling was first identified for its role in carcinogenesis, then for its function in embryonic development. The embryonic processes it controls include body axis patterning, cell fate specification, cell proliferation and cell migration. These processes are necessary for proper formation of important tissues including bone, heart and muscle. Its role in embryonic development was discovered when genetic mutations in Wnt pathway proteins produced abnormal fruit fly embryos. Wnt signaling also controls tissue regeneration in adult bone marrow, skin and intestine. Later research found that the genes responsible for these abnormalities also influenced breast cancer development in mice. This pathway's clinical importance was demonstrated by mutations that lead to various diseases, including breast and prostate cancer, glioblastoma, type II diabetes and others.

Wnt comprises a diverse family of secreted lipid-modified signaling glycoproteins that are 350-400 amino acids in length. The type of lipid modification that occurs on these proteins is palmitoylation of cysteines in a conserved pattern of 23-24 cysteine residues. Palmitoylation is necessary because it initiates targeting of the Wnt protein to the plasma membrane for secretion and it allows the Wnt protein to bind its receptor due to the covalent attachment of fatty acids. Wnt proteins also undergo glycosylation, which attaches a carbohydrate in order to ensure proper secretion. In Wnt signaling, these proteins act as ligands to activate the different Wnt pathways via paracrine and autocrine routes. These proteins are highly conserved across species. They can be found in mice, humans, Xenopus, zebrafish, Drosophila and many others.

Wnt Signaling

Wnt proteins are characterized by a high number of conserved cysteine residues. Although Wnt proteins carry an N-terminal signal peptide and are secreted, they are relatively insoluble due to a particular protein modification, cysteine palmitoylation, which is essential for Wnt function (Willert et al., 2003). The porcupine gene, which displays homology to acyl-transferases, and its worm homolog mom-1 are believed to encode the enzyme that is responsible for Wnt palmitoylation (Zhai et al., 2004). Other genes that are conserved and are essential for Wnt secretion, named wntless (wls) and evenness interrupted (evi), respectively. These genes encode a seven-pass transmembrane protein that is conserved from worms (mom-3) to man (hWLS).

Receptors, agonists, and antagonists for Wnts bind Frizzled (Fz) proteins, which are seven-pass transmembrane receptors with an extracellular N-terminal cysteine-rich domain (CRD) (Bhanot et al., 1996). The Wnt-Fz interaction appears promiscuous, in that a single Wnt can bind multiple Frizzled proteins (e.g., Bhanot et al., 1996) and vice versa. In binding Wnt, Fzs cooperate with a single-pass transmembrane molecule of the LRP family known as LRP5 and -6 in vertebrates (Pinson et al., 2000; Tamai et al., 2000). The transport of LRP5/6 to the cell surface is dependent on a chaperone called Mesd in mice (Culi and Mann, 2003; Hsieh et al., 2003). And consistent with a role of the Mesd chaperone in the transport of LRP5/6 transport, mutations in Mesd resemble loss of LRP5/6. Although it has not been formally demonstrated that Wnt molecules form trimeric complexes with LRP5/6 and Frizzled, surface expression of both receptors is required to initiate the Wnt signal.

Canonical Wnt Signaling

Once bound by their cognate ligands, the Fz/LRP coreceptor complex activates the canonical signaling pathway. Fz can physically interact with Dsh, a cytoplasmic protein that functions upstream of β-catenin and the kinase GSK-3. Wnt signaling controls phosphorylation of Dsh (reviewed in Wallingford and Habas, 2005). Recent studies have indicated that the coreceptor LRP5/6 interacts with Axin through five phosphorylated PPP(S/T)P repeats in the cytoplasmic tail of LRP (Davidson et al., 2005; Zeng et al., 2005). Wnts are thought to induce the phosphorylation of the cytoplasmic tail of LRP, thus regulating the docking of Axin. GSK3 phosphorylates the PPP(S/T) P motif, whereas caseine kinase I-γ (CK1γ) phosphorylates multiple motifs close to the GSK3 sites. CK1γ is unique within the CK1 family in that it is anchored in the membrane through C-terminal palmitoylation. Both kinases are essential for signal initiation.

Small molecule inhibitors of the mitogen-activated protein kinase (MEK) and glycogen synthesis kinase 3 (Gsk3) have been essential in the establishment and maintenance of embryonic stem cells (ESCs) from rats and from nonpermissive mouse strains. Inhibitors of Gsk-3 are known in the art, e.g., CHIR99021 (a Wnt pathway activator and inhibitor of Gsk3), the structure is provided below:

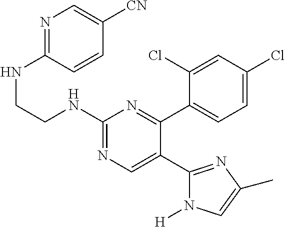 [SMILES: Cc1c[nH]c(-c2cnc(NCCNc3ccc(C#N)cn3)nc2-c2ccc(Cl)cc2Cl)n1]

Wnt Target Genes

Loss of components of the Wnt pathway can produce dramatic phenotypes that affect a wide variety of organs and tissues. A popular view equates Wnt signaling with maintenance or activation of stem cells (Reya and Clevers, 2005). It should be realized, however, that Wnt signals ultimately activate transcriptional programs and that there is no intrinsic restriction in the type of biological event that may be controlled by these programs.

Thus, Wnt signals can promote cell proliferation and tissue expansion but also control fate determination or terminal differentiation of postmitotic cells. Sometimes, these disparate events, proliferation and terminal differentiation, can be activated by Wnt in different cell types within the same structure, such as the hair follicle or the intestinal crypt (Reya and Clevers, 2005). Numerous Tcf target genes have been identified in diverse biological systems. These studies tend to focus on target genes involved in cancer, as exemplified by the wide interest in the Wnt target genes cMyc and Cyclin D1.

The Wnt pathway has distinct transcriptional outputs, which are determined by the developmental identity of the responding cell, rather than by the nature of the signal. In other words, the majority of Wnt target genes appear to be cell type specific. It is not clear whether “universal” Wnt/Tcf target genes exist. The best current candidates in vertebrates are Axin2/conductin (Jho et al., 2002) and SP5 (Weidinger et al., 2005). As noted (Logan and Nusse, 2004), Wnt signaling is autoregulated at many levels. The expression of a variety of positive and negative regulators of the pathway, such as Frizzleds, LRP and HSPG, Axin2, and TCF/Lef are all controlled by the β-catenin/TCF complex.

Patterning of the embryo and cell specification events are activated by a few evolutionarily conserved pathways, one of which is the Wnt/β-catenin pathway. These signaling proteins are used repeatedly during development and in diverse regions. The canonical Wnt pathway has been shown to regulate cell fate decisions, cell proliferation, and cell migration in the embryo. Canonical Wnt signaling is important for neural development, neural crest specification and differentiation, and cardiac development. The signals are transduced in a cell-context dependent manner to result in rapid changes in gene transcription. Reported evidence indicates that canonical Wnt signaling during narrow windows has differential effects during cardiac specification and heart development.

### β-Catenin

Beta-catenin (β-catenin) is a member of the plakophilin protein family. The plakophilins belong to the armadillo-related proteins, which are components of the desmosomal plaque. In addition to their adhesive function, the plakophilin β-catenins have been ascribed an important signaling function. For instance, β-catenin is a transcriptional co-activator of the T cell factor/lymphoid enhancer factor (TCF/LEF) complex that regulates embryonic, postnatal, and oncogenic growth in many tissues, including the heart (Brembeck et al. Curr Opin Genet Dev. 2006; 16:51-59). Cardiomyocyte growth occurs during left ventricular (LV) remodeling following chronic pressure overload and/or ischemic heart disease. Increased β-catenin levels were detected in the intercalated disc in heart specimens from patients with inherited cardiac hypertrophy (Masuelli et al. Cardiovasc Res. 2003; 60:376-387).

### HCN4

Potassium/sodium hyperpolarization-activated cyclic nucleotide-gated channel 4 is a protein that in humans is encoded by the HCN4 gene. Cellular automaticity and excitability in the cardiac conduction system result from activities of a diversity of ion channels. The pacemaker current (If or Ih) is encoded by the family of Hyperpolarization-activated, Cyclic Nucleotide gated (HCN) channels and plays a key role in the generation and autonomic regulation of sinus rhythm and rate. Four mammalian HCN isoforms (HCN1-4) have been identified, of which HCN4 is most abundantly expressed in the sinoatrial node. Previous studies have revealed early expression of HCN4 mRNA in a localized domain at cardiac crescent stages, and it specifically marks the SAN region during development and in adult. In addition, previous studies have observed, well before the coronary vascularization, the formation of a pacemaker region at the inflow tract of early heart tube.

HCN channels directly interact with intracellular cAMP so that an increase in cAMP levels results in increased If and more positive activation potentials. This increase thereby accelerates the heart rate (HR) in response to sympathetic stimulation. In contrast, muscarinic stimulation slows the heart rate in part due to a decrease in cAMP levels and a resulting reduction of If and more negative activation potentials. Ludwig, A. et al.; “Two pacemaker channels from human heart with profoundly different activation kinetics.” EMBO J. (1999) 18 (9):2323-2329. The importance of the HCN genes in regulating heart rate has recently been shown in a patient who suffered from mutation in his HCN4 gene. This mutation consisted of a complete deletion of the C-terminus of the gene which included the cAMP binding domain. This patient suffered from symptomatic bradycardia and an electronic pacemaker needed to implanted. These mutations were recreated in vitro experiments, and the mutated channel was expressed in a cell line. The mutated HCN4 channel was completely unresponsive to cAMP. See, J Clin Invest. 2003 May:111(10):1537-45.

An exemplary human HCN4 protein sequence is provided by GenBank Accession No: NP_005468 (SEQ ID NO: 5) and is as follows:

| | |
|---|---|
| 1 | mdklppsmrk rlyslpqqvg akawimdeee daeeegaggr qdpsrrsirl rplpspspsa |
| 61 | aaggtesrss algaadsegp argagksstn gdcrrfrgsl aslgsrgggs ggtgsgsshg |
| 121 | hlhdsaeerr liaegdaspg edrtppglaa eperpgasaq paasppppqg ppqpasasce |
| 181 | gpsvdtaikv eggaaagdqi lpeaevrlgq agfmgrqfga mlqpgvnkfs lrmfgsqkav |
| 241 | ereqervksa gfwiihpysd frfywdltml llmvgnliii pvgitffkde nttpwivfnv |
| 301 | vsdtfflidl vinfrtgivv ednteiildp qrikmkylks wfmvdfissi pvdyiflive |
| 361 | tridsevykt aralrivrft kilsllrllr lsrliryihq weeifhmtyd lasavvrivn |
| 421 | ligmmlllch wdgclqflvp mlqdfpddcw vsinnmvnns wgkqysyalf kamshmlcig |
| 481 | ygrqapvgms dvwltmlsmi vgatcyamfi ghataliqsl dssrrqygek ykgvegymsf |
| 541 | hklppdtrqr ihdyyehryq gkmfdeesil gelseplree iinfncrklv asmplfanad |
| 601 | pnfvtsmltk lrfevfqpgd yiiregtigk kmyfiqhgvv svltkgnket kladgsyfge |
| 661 | iclltrgrrt asvradtycr lyslsvdnfn evleeypmmr rafetvaldr ldrigkknsi |
| 721 | llhkvqhdln sgvfnyqene iiggivqhdr emahcahrvq aaasatptpt pviwtpliqa |
| 781 | plqaaaatts vaialthhpr lpaaifrppp gsglgnlgag qtprhlkrlq slipsalgsa |
| 841 | spasspsqvd tpssssfhiq qlagfsapag lspllpssss spppgacgsp saptpsagva |
| 901 | attiagfghf hkalggslss sdsplltplq pgarspqaaq pspappgarg glglpehflp |
| 961 | pppssrspss spgqlgqppg elslglatgp lstpetpprq peppslvaga sggaspvgft |
| 1021 | prgglsppgh spgpprtfps apprasgshg slllppassp pppqvpqrrg tppltpgrlt |
| 1081 | qdlklisasq palpgdgagt lrrasphssg esmaafplfp ragggsggsg ssgglgppgr |
| 1141 | pygaipgqhv tlprktssgs lppplslfga ratssggppl tagpqrepga rpepvrsklp |
| 1201 | snl |

Non-human orthologs of HCN4, including the mouse, rat, and chicken orthologs, are identified in NCBI. In certain embodiments, at least 75%, 80%, 85%, 90%, 95%, 98%, or more of the cells in the isolated population of CCS progenitor cells express HCN4 (i.e., are HCN4+).

An exemplary human HCN4 nucleotide sequence is provided by GenBank Accession No: NM_005477.2 (SEQ ID NO: 6) and is as follows. The start and stop codons are bold and underlined.

| | |
|---|---|
| 1 | caaaaatgcc agggaaaggc gagcccagag cttggtgatg gagaaattgg gaagccaccc |
| 61 | cccacccttc aatcttagga tggggaattc gcaactgaag ccggagcttc agacttgggg |
| 121 | cgcactccca gcttagccca ggaaagagat ttaagggcgc agcagtgtgg atacctctca |
| 181 | ccccggcccc gaaggtctag cgagggtcta acctgggccc cttgccaggc ccgccccccg |
| 241 | cccctttcca gcccccggcc cgtgcgccgc tgccccttta agaagcccag gtaggcaggc |
| 301 | ccggctgctg gagccgctcc tatggcaacc cgcgagctgc ggcggcttca tgaatattcc |
| 361 | ggggcgcggg agcccgagcg ctgccggagg gcgcttcggg ggaggcggcc gctgatgtaa |
| 421 | gcccggcggg tcgctgggct ccgctcggtt gcggcgggag ccccgggacg ggccggacgg |
| 481 | gccggggcag aggaggcgag gcgagctcgc gggtggccag ccacaaagcc cgggcggcga |
| 541 | gacagacgga cagccagccc tcccgcggga cgcacgcccg ggacccgcgc gggccgtgcg |
| 601 | ctctgcactc cggagcggtt ccctgagcgc cgcggccgca gagcctctcc ggccggcgcc |
| 661 | cattgttccc cgcgggggcg gggcgcctgg agccgggcgg cgcgccgcgc ccctgaacgc |
| 721 | cagagggagg gagggaggca agaagggagc gcggggtccc cgcgcccagc cgggcccggg |
| 781 | aggaggtgta gcgcggcgag cccggggact cggagcggga ctaggatcct ccccgcggcg |
| 841 | cgcagcctgc ccaagcatgg gcgcctgagg ctgcccccac gccggcggca aaggacgcgt |
| 901 | ccccacgggc ggactgaccg gcgggcggac ctggagcccg tccgcggcgc cgcgctcctg |
| 961 | cccccggccc ggtccgaccc cggcccctgg cgccatggac aagctgccgc cgtccatgcg |
| 1021 | caagcggctc tacagcctcc cgcagcaggt gggggccaag gcgtggatca tggacgagga |
| 1081 | agaggacgcc gaggaggagg gggccggggg ccgccaagac cccagccgca ggagcatccg |
| 1141 | gctgcggcca ctgccctcgc cctccccctc ggcggccgcg ggtggcacgg agtcccggag |
| 1201 | ctcggccctc ggggcagcgg acagcgaagg gccggcccgc ggcgcgggca agtccagcac |
| 1261 | gaacggcgac tgcaggcgct tccgcgggag cctggcctcg ctgggcagcc ggggcggcgg |
| 1321 | cagcggcggc acggggagcg gcagcagtca cggacacctg catgactccg cggaggagcg |
| 1381 | gcggctcatc gccgagggcg acgcgtcccc cggcgaggac aggacgcccc caggcctggc |
| 1441 | ggccgagccc gagcgccccg gcgcctcggc gcagcccgca gcctcgccgc cgccgcccca |
| 1501 | gcagccaccg cagccggcct ccgcctcctg cgagcagccc tcggtggaca ccgctatcaa |
| 1561 | agtggaggga ggcgcggctg ccggcgacca gatcctcccg gaggccgagg tgcgcctggg |
| 1621 | ccaggccggc ttcatgcagc gccagttcgg ggccatgctc caacccgggg tcaacaaatt |
| 1681 | ctccctaagg atgttcggca gccagaaagc cgtggagcgc gaacaggaga gggtcaagtc |
| 1741 | ggccggattt tggattatcc acccctacag tgacttcaga ttttactggg acctgaccat |
| 1801 | gctgctgctg atggtgggaa acctgattat cattcctgtg ggcatcacct tcttcaagga |
| 1861 | tgagaacacc acaccctgga ttgtcttcaa tgtggtgtca gacacattct tcctcatcga |
| 1921 | cttggtcctc aacttccgca cagggatcgt ggtggaggac aacacagaga tcatcctgga |
| 1981 | cccgcagcgg attaaaatga agtacctgaa aagctggttc atggtagatt tcatttcctc |
| 2041 | catccccgtg gactacatct tcctcattgt ggagacacgc atcgactcgg aggtctacaa |
| 2101 | gactgcccgg gccctgcgca ttgtccgctt cacgaagatc ctcagcctct tacgcctgtt |
| 2161 | acgcctctcc cgcctcattc gatatattca ccagtgggaa gagatcttcc acatgaccta |
| 2221 | cgacctggcc agcgccgtgg tgcgcatcgt gaacctcatc ggcatgatgc tcctgctctg |
| 2281 | ccactgggac ggctgcctgc agttcctggt acccatgcta caggacttcc ctgacgactg |
| 2341 | ctgggtgtcc atcaacaaca tggtgaacaa ctcctggggg aagcagtact cctacgcgct |
| 2401 | cttcaaggcc atgagccaca tgctgtgcat cggctacggg cggcaggcgc ccgtgggcat |
| 2461 | gtccgacgtc tggctcacca tgctcagcat gatcgtgggt gccacctgct acgccatgtt |
| 2521 | cattggccac gccactgccc tcatccagtc cctggactcc tcccggcgcc agtaccagga |
| 2581 | aaagtacaag caggtggagc agtacatgtc ctttcacaag ctcccgcccg acacccggca |
| 2641 | gcgcatccac gactactacg agcaccgcta ccagggcaag atgttcgacg aggagagcat |
| 2701 | cctgggcgag ctaagcgagc ccctgcggga ggagatcatc aactttaact gtcggaagct |
| 2761 | ggtggcctcc atgccactgt ttgccaatgc ggaccccaac ttcgtgacgt ccatgctgac |
| 2821 | caagctgcgt ttcgaggtct tccagcctgg ggactacatc atccgggaag gcaccattgg |
| 2881 | caagaagatg tacttcatcc agcatggcgt ggtcagcgtg ctcaccaagg gcaacaagga |
| 2941 | gaccaagctg gccgacggct cctactttgg agagatctgc ctgctgaccc ggggccggcg |
| 3001 | cacagccagc gtgagggccg acacctactg ccgcctctac tcgctgagcg tggacaactt |
| 3061 | caatgaggtg ctggaggagt accccatgat gcgaagggcc ttcgagaccg tggcgctgga |
| 3121 | ccgcctggac cgcattggca agaagaactc catcctcctc cacaaagtcc agcacgacct |
| 3181 | caactccggc gtcttcaact accaggagaa tgagatcatc cagcagattg tgcagcatga |
| 3241 | ccgggagatg gcccactgcg cgcaccgcgt ccaggctgct gcctctgcca ccccaacccc |
| 3301 | cacgcccgtc atctggaccc cgctgatcca ggcaccactg caggctgccg ctgccaccac |
| 3361 | ttctgtggcc atagccctca cccaccaccc tcgcctgcct gctgccatct tccgccctcc |
| 3421 | cccaggatct gggctgggca acctcggtgc cgggcagacg ccaaggcacc tgaaacggct |
| 3481 | gcagtccctg atcccttctg cgctgggctc cgcctcgccc gccagcagcc cgtcccaggt |
| 3541 | ggacacaccg tcttcatcct ccttccacat ccaacagctg gctggattct ctgcccccgc |
| 3601 | tggactgagc ccactcctgc cctcatccag ctcctcccca ccccccgggg cctgtggctc |
| 3661 | cccctcggct cccacaccat cagctggcgt agccgccacc accatagccg ggtttggcca |
| 3721 | cttccacaag gcgctgggtg gctccctgtc ctcctccgac tctcccctgc tcaccccgct |
| 3781 | gcagccaggc gcccgctccc cgcaggctgc ccagccatct cccgcgccac ccggggcccg |
| 3841 | gggaggcctg ggactcccgg agcacttcct gccaccccca ccctcatcca gatccccgtc |
| 3901 | atctagcccc gggcagctgg gccagcctcc cggggagttg tccctaggtc tggccactgg |
| 3961 | cccactgagc acgccagaga cacccccacg gcagcctgag ccgccgtccc ttgtggcagg |
| 4021 | ggcctctggg ggggcttccc ctgtaggctt tactccccga ggaggtctca gcccccctgg |
| 4081 | ccacagccca ggccccccaa gaaccttccc gagtgccccg ccccgggcct ctggctccca |
| 4141 | cggatccttg ctcctgccac ctgcatccag ccccccacca ccccaggtcc cccagcgccg |
| 4201 | gggcacaccc ccgctcaccc ccggccgcct cacccaggac ctcaagctca tctccgcgtc |
| 4261 | tcagccagcc ctgcctcagg acggggcgca gactctccgc agagcctccc cgcactcctc |
| 4321 | aggggagtcc atggctgcct tcccgctctt ccccagggct gggggtggca gcgggggcag |
| 4381 | tgggagcagc gggggcctcg gtccccctgg gaggccctat ggtgccatcc ccggccagca |
| 4441 | cgtcactctg cctcggaaga catcctcagg ttctttgcca ccccctctgt ctttgtttgg |
| 4501 | ggcaagagcc acctcttctg gggggccccc tctgactgct ggaccccaga gggaacctgg |
| 4561 | ggccaggcct gagccagtgc gctccaaact gccatccaat ctatgagctg ggcccttcct |
| 4621 | tccctcttct ttcttctttt ctctcccttc cttcttcctt caggtttaac tgtgattagg |
| 4681 | agatatacca ataacagtaa taattattta aaaaaccaca cacaccagaa aaacaaaaga |
| 4741 | cagcagaaaa taaccaggta ttcttagagc tatagatttt tggtcacttg cttttataga |
| 4801 | ctattttaat actcagcact agagggaggg agggggaggg aggagggagc aggcaggtcc |
| 4861 | caaatgcaaa agccagagaa aggcagatgg ggtctccggg gctgggcagg ggtgggagtg |
| 4921 | gccagtgttg gcggttctta gagcagatgt gtcattgtgt tcatttagag aaacagctgc |
| 4981 | catcagcccg ttagctgtaa cttggagctc cactctgccc ccagaaaggg gctgccctgg |
| 5041 | ggtgtgccct ggggagcctc agaagcctgc gaccttggga gaaaagggcc agggccctga |
| 5101 | gggcctagca ttttttctac tgtaaacgta gcaagatctg tatatgaata tgtatatgta |
| 5161 | tatgtatgta agatgtgtat atgtatagct atgtagcgct ctgtagagcc atgtagatag |
| 5221 | ccactcacat gtgcgcacac gtgtgcggtc tagtttaatc ccatgttgac aggatgccca |
| 5281 | ggtcacctta cacccagcaa cccgccttgg cccacaggct gtgcactgca tggtctaggg |
| 5341 | acgttctctc tccagtcctc agggaagagg accccaggac ttcgcagcag gccccctctc |
| 5401 | tccccatctc tggtctcaaa gccagtccca gcctgacctc tcaccacacg gaagtggaag |
| 5461 | actccccttt cctagggcct caagcacaca ccgccacctc tggggccgtc agtttgccca |
| 5521 | tctgtacagt gggaggtgag cggaacttct gtttattgag tctgctctgt gccaagcact |
| 5581 | ggtttcgcac tttacacaca ttaactcctt cagtttcaca aagaccatgg ggtgggtact |
| 5641 | ttgattctcc ccatttagca gaggaagaaa cagttttggg taatttttcc agaatcatgt |
| 5701 | aactaggagt ggcagagtgg ggactgattt gaggttcgag tccacgcctc cttgaggccc |
| 5761 | aagtctgtgt tccttccatc agaaaactgt gttgaggggg gctgaggtag atggtcccca |
| 5821 | agcatggtac agaaggaaga caccagattt tggcagcagt caggcctggg tttgaatccc |
| 5881 | agccctgcca cttcttagct gtatgatctt gggcaagtta tctgaccttt ctgtcacctc |
| 5941 | atttgtaaaa tgggaataat tatggtactg cctcacaagg acctatgagg accagatgag |
| 6001 | aaaaatctat atgtgaaatg cccagcccag cgcctggcac ataccatggt aggtgctcaa |
| 6061 | taaaaaatca catttcttct gcccctcata tgcccagcct attgctccag caaactatgt |
| 6121 | gagagcccag ggagctttgg ctgagggctc caagacttaa aatctcagga ctcaggaggt |
| 6181 | ggctgggcct ccctaagggc ccaaggaagg tgtgtggcca gaggtgggtg ggagccaggc |
| 6241 | cttgagaagt gggaagactt caacagggag agagggaggg aatggtgggt gggatggagt |
| 6301 | gtatggtggg gagattcctg aggtggatgt ggagtggtgg atcagggctt tgggagggga |
| 6361 | tccccaggct gaggggtcag agggacggcc ttgggtgata gggtaaggga ttgtctgggc |
| 6421 | ttagtcctgg caactaggag ccataagcag gttccagatt gcgggaacga gaaagcagct |
| 6481 | cagatgcctt tggaggcacc atcctccctc ctcccagatg ggatcttgcc agagccaagg |
| 6541 | tcaggggtct gcccctgcct atagggccag agcaggtatg gctgcaatcc ccaagtaatg |
| 6601 | agaagggctg gtcccacatt atccatccag aaccttccat gctccaagcc agaatgttgg |
| 6661 | caagatcggg ttttgccttg agctatcctg ggatgtgaga caaaccgatt tctccataga |
| 6721 | tgggctgcag ggagtgggag gcagtactcc aggagagaag tgggtgaagg ttcctgggat |
| 6781 | cttaggtaaa gactagacgc cgcctagtac tggtctctac tgtgctggct caggagttct |
| 6841 | gagaactgga aggacttagc ctcaacctga gttctgcaca caccccttcc ccttaaggaa |
| 6901 | ggcagctctg agaggcagca ggacttgatc caaacccaca gtcttgtcct ggaggcagca |
| 6961 | ggggtgaagg tggagggtcc agggccatga ggagccccct tgccatcaga gcctggccta |
| 7021 | accaccctct tctctactta cacacacatg cattttataa tagctctgac ccaacctggc |
| 7081 | cactctgcag agactgggac agacaggtgc aggcaatggg ccctcccaca cccagtcacc |
| 7141 | tacaaggaat tttcaaatcc acttttaaaa cagaaaccgg taaatgcgcc gtattgtata |
| 7201 | ttttatttaa ataaaaaaaa ttccagcaaa aaaaaaaaaa aaaaa |

### Islet1 (Isl1)

Isl1 is a pan-cardiac progenitor marker expressed in both first and second heart fields. It also has a biological function as shown in Isl1 knockout mice which have a severely deformed heart. More recently it has been defined as a marker for a cardiac progenitor cell lineage that is capable of differentiating into all 3 major cell types of the heart: cardiomyocytes, smooth muscle and endothelial cell lineages.

A multipotent islet 1 (isl1+) cardiovascular progenitor (MICP) is able to give rise to the major three cell types of the heart: cardiomyocytes, smooth muscles and endothelial cells, and has clonogenic and self-renewing ability (Laugwitz et al., 2005; Moretti et al., 2006). In Isl1 knockout mice, histological analysis of mutant hearts between embryonic day (ED) 9.0 and ED9.5 showed a misshapen single heart ventricle as the cause of death (Cai et al., 2003). Lineage tracing studies in mice document that isl1+ progenitors give rise to most of the cells in the heart, mostly on the right side, including most of the conduction system: the sinoatrial (SA) node, the atrioventricular (AV) node, His-bundle, and Purkinje fiber complex (Cai et al, 2003; Laugwitz et al., 2005; Moretti et al., 2006; Sun et al., 2007). Disruption of development, differentiation or maturation of any of these components can lead to arrhythmias such as sinus arrest, AV block, ventricular tachycardia and sudden death (Bruneau et al., 2001).

An exemplary human Isl1 protein sequence is provided by GenBank Accession No: NP_002193.2 (SEQ ID NO: 7) and is as follows:

| | |
|---|---|
| 1 | mgdmgdppkk krlislcvgc gnqihdqyil rvspdlewha aclkcaecnq yldesctcfv |
| 61 | rdgktyckrd yirlygikca kcsigfsknd fvmrarskvy hiecfrcvac srqlipgdef |
| 121 | alredglfcr adhdvveras lgagdplspl hparplqmaa episarqpal rphvhkqpek |
| 181 | ttrvrtvine kqlhtlrtcy aanprpdalm keqlvemtgl sprvirvwfq nkrckdkkrs |
| 241 | immkqlqqqg pndktniqgm tgtpmvaasp erhdgglqan pvevqsyqpp wkvlsdfalq |
| 301 | sdidgpafgq lvnfseggpg snstgsevas mssqlpdtpn smvaspiea |

An exemplary human Isl1 nucleic acid sequence is provided by GenBank Accession No: NM_002202.2 (SEQ ID NO: 8) and is as follows. The start and stop codons are bold and highlighted.

| | |
|---|---|
| 1 | gaaggaagag gaagaggagg agagggaggc cagagccaga acagcccggc agcccgagct |
| 61 | tcgggggaga acggcctgag ccccgagcaa gttgcctcgg gagccctaat cctctcccgc |
| 121 | tggctcgccg agcggtcagt ggcgctcagc ggcggcgagg ctgaaatatg ataatcagaa |
| 181 | cagctgcgcc gcgcgccctg cagccaatgg gcgcggcgct cgcctgacgt ccccgcgcgc |
| 241 | tgcgtcagac caatggcgat ggagctgagt tggagcagag aagtttgagt aagagataag |
| 301 | gaagagaggt gcccgagccg cgccgagtct gccgccgccg cagcgcctcc gctccgccaa |
| 361 | ctccgccggc ttaaattgga ctcctagatc cgcgagggcg cggcgcagcc gagcagcggc |
| 421 | tctttcagca ttggcaaccc caggggccaa tatttcccac ttagccacag ctccagcatc |
| 481 | ctctctgtgg gctgttcacc aactgtacaa ccaccatttc actgtggaca ttactccctc |
| 541 | ttacagatat gggagacatg ggagatccac caaaaaaaaa acgtctgatt tccctatgtg |
| 601 | ttggttgcgg caatcagatt cacgatcagt atattctgag ggtttctccg gatttggaat |
| 661 | ggcatgcggc atgtttgaaa tgtgcggagt gtaatcagta tttggacgag agctgtacat |
| 721 | gctttgttag ggatgggaaa acctactgta aaagagatta tatcaggttg tacgggatca |
| 781 | aatgcgccaa gtgcagcatc ggcttcagca agaacgactt cgtgatgcgt gcccgctcca |
| 841 | aggtgtatca catcgagtgt ttccgctgtg tggcctgcag ccgccagctc atccctgggg |
| 901 | acgaatttgc gcttcgggag gacggtctct tctgccgagc agaccacgat gtggtggaga |
| 961 | gggccagtct aggcgctggc gacccgctca gtcccctgca tccagcgcgg ccactgcaaa |
| 1021 | tggcagcgga gcccatctcc gccaggcagc cagccctgcg gccccacgtc cacaagcagc |
| 1081 | cggagaagac cacccgcgtg cggactgtgc tgaacgagaa gcagctgcac accttgcgga |
| 1141 | cctgctacgc cgcaaacccg cggccagatg cgctcatgaa ggagcaactg gtagagatga |
| 1201 | cgggcctcag tccccgtgtg atccgggtct ggtttcaaaa caagcggtgc aaggacaaga |
| 1261 | agcgaagcat catgatgaag caactccagc agcagcagcc caatgacaaa actaatatcc |
| 1321 | aggggatgac aggaactccc atggtggctg ccagtccaga gagacacgac ggtggcttac |
| 1381 | aggctaaccc agtggaagta caaagttacc agccaccttg gaaagtactg agcgacttcg |
| 1441 | ccttgcagag tgacatagat cagcctgctt ttcagcaact ggtcaatttt tcagaaggag |
| 1501 | gaccgggctc taattccact ggcagtgaag tagcatcaat gtcctctcaa cttccagata |
| 1561 | cacctaacag catggtagcc agtcctattg aggcatgagg aacattcatt ctgtattttt |
| 1621 | tttccctgtt ggagaaagtg ggaaattata atgtcgaact ctgaaacaaa agtatttaac |
| 1681 | gacccagtca atgaaaactg aatcaagaaa tgaatgctcc atgaaatgca cgaagtctgt |
| 1741 | tttaatgaca aggtgatatg gtagcaacac tgtgaagaca atcatgggat tttactagaa |
| 1801 | ttaaacaaca aacaaaacgc aaaacccagt atatgctatt caatgatctt agaagtactg |
| 1861 | aaaaaaaaag acgtttttaa aacgtagagg atttatattc aaggatctca aagaaagcat |
| 1921 | tttcatttca ctgcacatct agagaaaaac aaaaatagaa aattttctag tccatcctaa |
| 1981 | tctgaatggt gctgtttcta tattggtcat tgccttgcca aacaggagct ccagcaaaag |
| 2041 | cgcaggaaga gagactggcc tccttggctg aaagagtcct ttcaggaagg tggagctgca |
| 2101 | ttggtttgat atgtttaaag ttgactttaa caaggggtta attgaaatcc tgggtctctt |
| 2161 | ggcctgtcct gtagctggtt tattttttac tttgccccct ccccactttt tttgagatcc |
| 2221 | atcctttatc aagaagtctg aagcgactat aaaggttttt gaattcagat ttaaaaacca |
| 2281 | acttataaag cattgcaaca aggttacctc tattttgcca caagcgtctc gggattgtgt |
| 2341 | ttgacttgtg tctgtccaag aacttttccc ccaaagatgt gtatagttat tggttaaaat |
| 2401 | gactgttttc tctctctatg gaaataaaaa ggaaaaaaaa aaaggaaact ttttttgttt |
| 2461 | gctcttgcat tgcaaaaatt ataaagtaat ttattattta ttgtcggaag acttgccact |
| 2521 | tttcatgtca tttgacattt tttgtttgct gaagtgaaaa aaaaagataa aggttgtacg |
| 2581 | gtggtctttg aattatatgt ctaattctat gtgttttgtc tttttcttaa atattatgtg |
| 2641 | aaatcaaagc gccatatgta gaattatatc ttcaggacta tttcactaat aaacatttgg |
| 2701 | catagataaa taaataaaaa aaaaaaaaa |

### Pharmaceutical Compositions

In certain embodiments, the present invention provides for a pharmaceutical composition comprising an agent employed in the present invention. The agent can be suitably formulated and introduced into a subject or the environment of a cell by any means recognized for such delivery.

Such compositions typically include the agent and a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions.

A pharmaceutical composition is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in a selected solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

The compositions of the invention could also be formulated as nanoparticle formulations. The compounds of the invention can be administered for immediate-release, delayed-release, modified-release, sustained-release, pulsed-release and/or controlled-release applications. The pharmaceutical compositions of the invention may contain from 0.01 to 99% weight—per volume of the active material. For administration by inhalation, the compounds are delivered in the form of an aerosol spray from pressured container or dispenser which contains a suitable propellant, e.g., a gas such as carbon dioxide, or a nebulizer. Such methods include those described in U.S. Pat. No. 6,468,798.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art. The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For a compound used in a method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of an agent (i.e., an effective dosage) depends on the agent selected. For instance, single dose amounts of an agent in the range of approximately 1 pg to 1000 mg may be administered; in some embodiments, 10, 30, 100, or 1000 pg, or 10, 30, 100, or 1000 ng, or 10, 30, 100, or 1000 pg, or 10, 30, 100, or 1000 mg may be administered. In some embodiments, 1-5 g of the compositions can be administered.

A therapeutically effective amount of the compound of the present invention can be determined by methods known in the art. In addition to depending on the agent and selected/pharmaceutical formulation used, the therapeutically effective quantities of a pharmaceutical composition of the invention will depend on the age and on the general physiological condition of the patient and the route of administration. In certain embodiments, the therapeutic doses will generally be between about 10 and 2000 mg/day and preferably between about 30 and 1500 mg/day. Other ranges may be used, including, for example, 50-500 mg/day, 50-300 mg/day, 100-200 mg/day.

Administration may be once a day, twice a day, or more often, and may be decreased during a maintenance phase of the disease or disorder, e.g. once every second or third day instead of every day or twice a day. The dose and the administration frequency will depend on the clinical signs, which confirm maintenance of the remission phase, with the reduction or absence of at least one or more preferably more than one clinical signs of the acute phase known to the person skilled in the art. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of an agent can include a single treatment or, optionally, can include a series of treatments.

It can be appreciated that the method of introducing an agent into the environment of a cell will depend on the type of cell and the makeup of its environment. Suitable amounts of an agent must be introduced and these amounts can be empirically determined using standard methods. Exemplary effective concentrations of an individual agent in the environment of a cell can be 500 millimolar or less, 50 millimolar or less, 10 millimolar or less, 1 millimolar or less, 500 nanomolar or less, 50 nanomolar or less, 10 nanomolar or less, or even compositions in which concentrations of 1 nanomolar or less can be used.

Pharmaceutical compositions may be assembled into kits or pharmaceutical systems for use in treating heart disease, e.g., congenital heart disease. Kits or pharmaceutical systems according to this aspect of the invention comprise a carrier means, such as a box, carton, tube, having in close confinement therein one or more container means, such as vials, tubes, ampoules, bottles, syringes, or bags. The kits or pharmaceutical systems of the invention may also comprise associated instructions for using the kit. The pharmaceutical compositions can be included in a kit, container, pack, or dispenser together with instructions for administration.

### Kits and Instructions

Provided are kits comprising compositions and methods of the invention, including instructions for use thereof, including kits comprising cells, expression vehicles (e.g., recombinant viruses, vectors) and the like.

For example, in alternative embodiments, provided are kits comprising compositions used to practice this invention. In one aspect, the kit further comprising instructions for practicing any methods of the invention, e.g., in vitro or ex vivo methods.

### EXAMPLES

The following examples are put forth so as to provide those of ordinary skill in the art with a complete disclosure and description of how to make and use the assay, screening, and therapeutic methods of the invention, and are not intended to limit the scope of what the inventors regard as their invention.

### Example 1: FHF/SHF-Like Cells were Induced in Spheroid PSC Culture

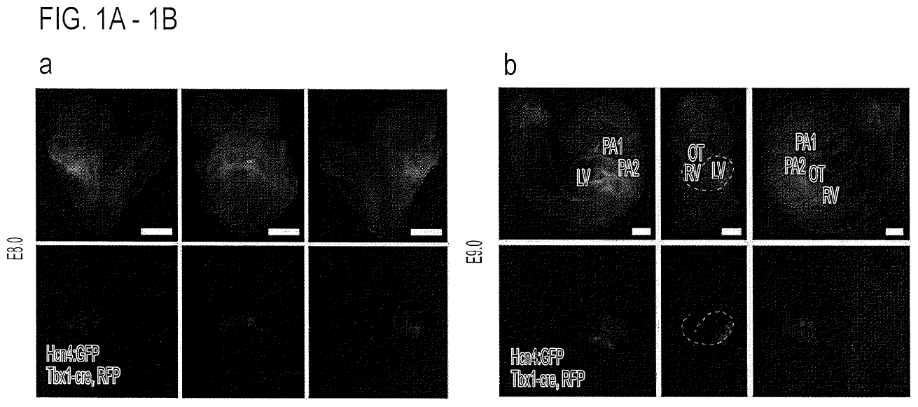

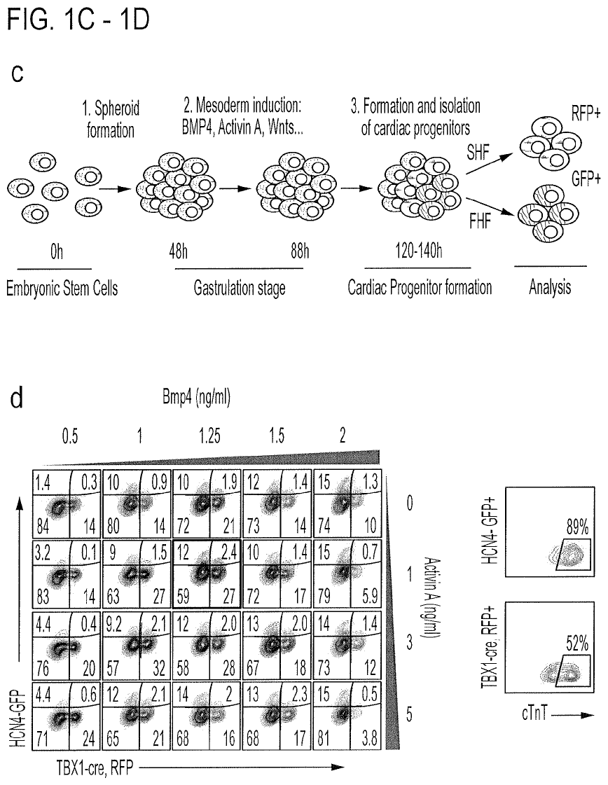

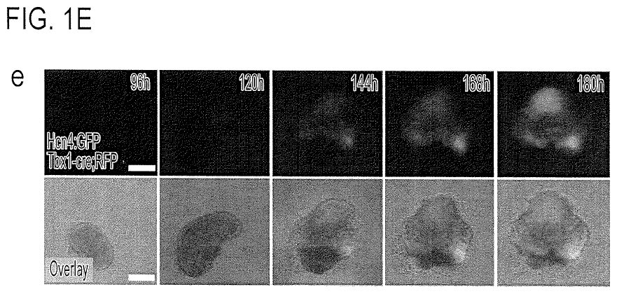

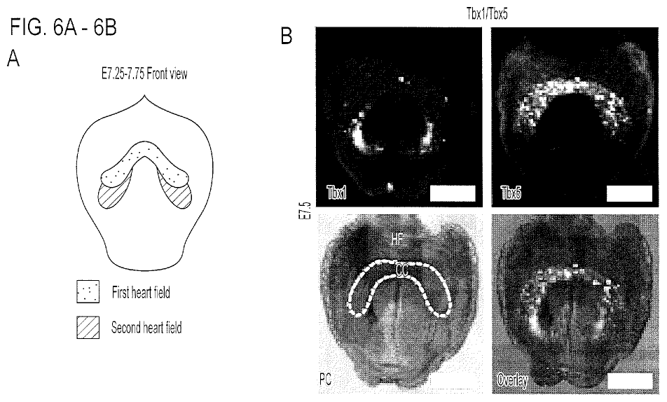

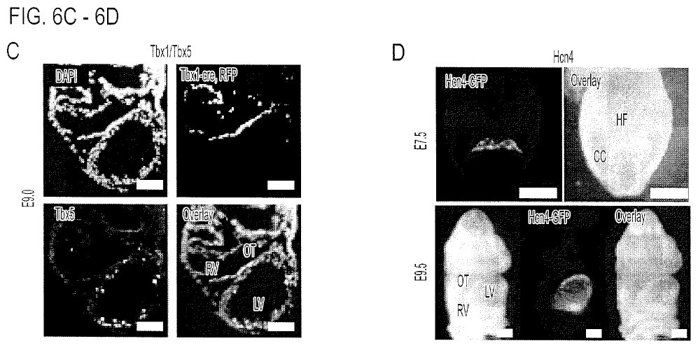

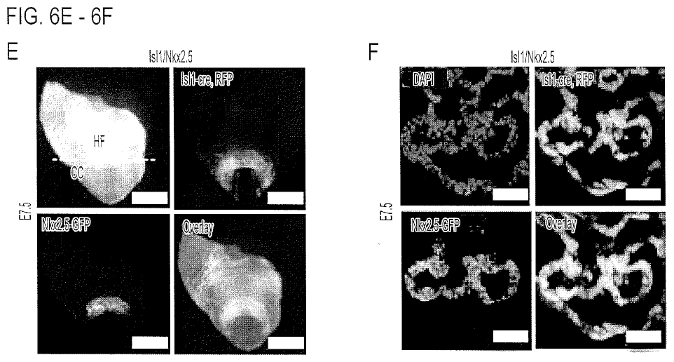

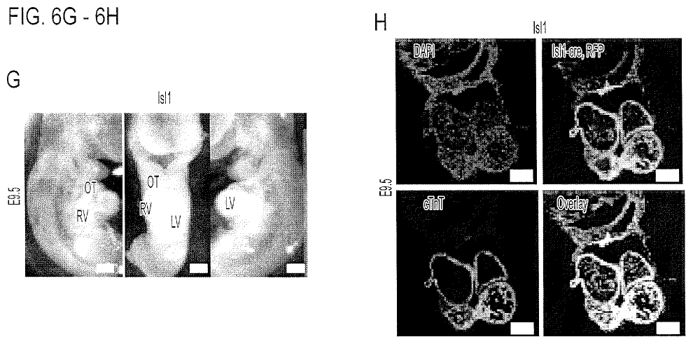

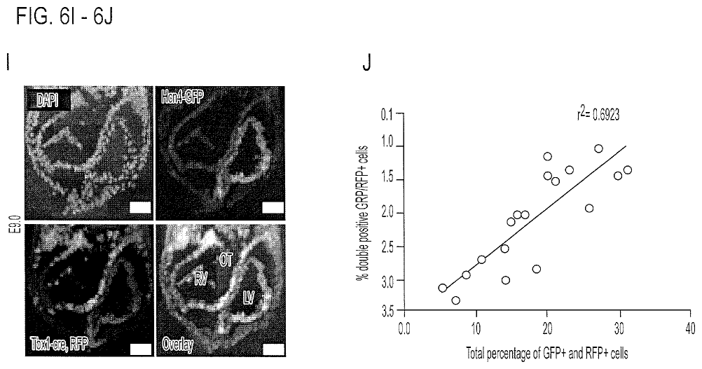

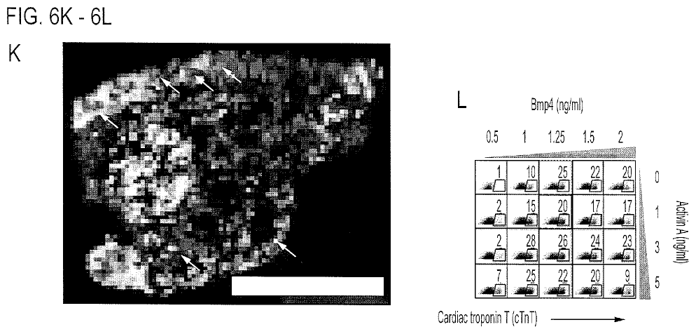

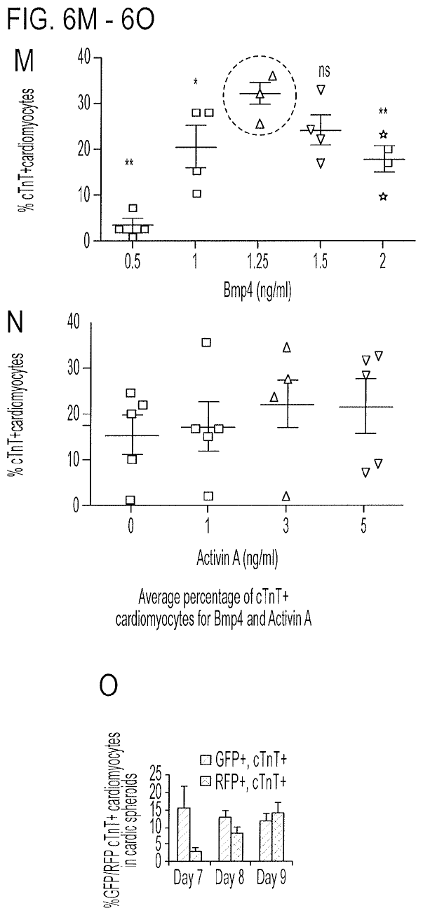

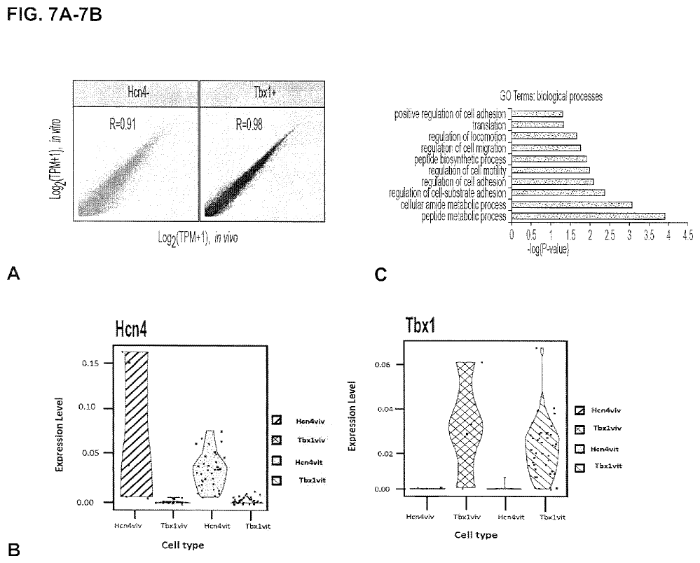

Lineage tracing experiments with CPC markers, including Hcn4, Tbx5, Isl1, and Tbx1, have identified distinct FHF and SHF structures in developing mouse embryos. To verify if these markers faithfully label the FHF or the SHF, their expression was examined in mice between embryonic days 7.5 and 9.5 post fertilization (E7.5 and E9.5). Hcn4 and Tbx5 were both expressed in the FHF (FIG. 6A-6D), and Tbx1 was expressed in the SHF and structures derived thereof (FIGS. 6A-6C and 6I). When traced with Isl1^{Cre} mice³⁷, cells expressing Isl1, regarded as a SHF marker, gave rise to both FHF and SHF structures (FIG. 6E-6H), including the entire LV at E9.5. Isl1 lineage tracing further revealed that Nkx2.5-expressing cells in the cardiac crescent were derived from Isl1+ cells (FIGS. 6E and 6F). This suggests that Isl1 marked undifferentiated CPCs of both heart fields. Based on these analyses, mice were generated expressing green/red fluorescent protein (GFP/RFP) in FHF cells/SHF lineage cells by crossing Tbx1cre; Ai9 mice with Hcn4^{GFP} mice^{17, 38}. In this system, GFP was expressed in Hcn4+ cells in the FIF¹⁵ and RFP permanently marks Tbx1 progeny in the SHF¹⁷. GFP was expressed in the cardiac crescent, whereas RFP labeled the region dorsal to the crescent where SHF cells were located (FIG. 1A). At E9.0, GFP was expressed in the LV, and RFP was restricted to the pharyngeal mesoderm and the OT/RV (FIG. 1B and FIG. 1I), confirming that GFP and RFP marked FHF cells and SHF cells, respectively.

Next, an embryonic stem cell (ESC) line (ESCHcn4-GFP; Tbx1-Cre Ai9) was established from the mice to determine if heart field specification can be recapitulated in a PSC system. It was hypothesized that a 3D multicellular system would better resemble heart field development in vivo, as early development is a highly dynamic process that involves tissue-tissue interactions between multiple cell types. Since CPCs are specified during mid-late gastrulation 7, 8, multicellular 3D spheroids were generated with the PSCs and treated them with various concentrations of Activin A and Bmp4 to determine whether induction of the early mesoderm influences heart field specification (FIGS. 1C and 1D). After 5 days of differentiation (120 h), GFP+ and RFP+ cells started to appear in the spheroids. The spheroids were analyzed for GFP+ and RFP+ cells by fluorescent activated cell sorting (FACS) at day 5.5 (132 h). The FACS data showed that cells were either GFP+ or RFP+ with <4% double positive (GFP+/RFP+) cells among all fluorescent cells (FIG. 1D), indicating that FHF and SHF cells were distinctively specified in cardiac spheroids. The double positive GFP+/RFP+ cells correlated with the total number of GFP+ and RFP+ cells (FIG. 6J) and were sporadically interspersed within the GFP+ domain (FIG. 6K).

The overall effects on cardiogenesis were evaluated by analyzing the number of cardiomyocytes at day 9 (FIG. 6L). Varying Bmp4 concentrations had a profound effect on cardiogenesis with the number of cTnT+ cardiomyocytes reaching >30% with 1.25 ng/ml Bmp4, more than a 10-fold difference compared to 0.5 ng/ml Bmp4, whereas increasing Activin A levels had a modest effect on cardiogenesis compared to control (FIGS. 6M and 6N), indicating an important role for Bmp signaling during early cardiogenesis.

To determine the individual cardiomyogenic potential of GFP+ and RFP+ cells, the cardiomyocyte contribution was analyzed from the most cardiogenic condition (1.25 ng/ml Bmp4, 1 ng/ml Activin A) at day 9. 89% of GFP⁺ cells were positive for cTnT, and showed a differentiation bias towards a cardiomyogenic cell fate, whereas 52% of RFP+ cells were positive for cTnT (FIG. 1D). This indicated that GFP+ cells were primarily unipotent and cardiomyogenic, whereas RFP+ cells likely gave rise to several cardiac cell lineages.

To monitor the process of GFP+/RFP+ cell induction, a time-lapse analysis was performed of the spheroids (FIG. 1E). At 120 hours (5 days) of differentiation, areas of GFP+ and RFP+ cells started to appear adjacent to each other. GFP+ zones generally appeared in the periphery of cardiac spheroids, whereas RFP+ zones appeared more central (FIG. 6K). After 168 h (7 days), the majority of cardiomyocytes (cTnT+ cells) in the cardiac spheroids were GFP+, whereas RFP+ cardiomyocytes continuously increased between 168 h-204 h (7-9 days) (FIG. 6O), demonstrated that cardiomyogenesis was delayed in RFP+ CPCs compared to GFP+ CPCs, similar to in vivo, where the SHF did not contribute to the myocardium until the looping stage (E8.5)³⁹. It is worth noting that both populations maintained the complementary pattern over time within the spheroids (FIG. 1E), analogous to developing heart fields in vivo.

### Example 2: PSC-Derived FHF/SHF Progenitors were Similar to Endogenous FHF/SHF Progenitors in Gene Expression and Differentiation Potential

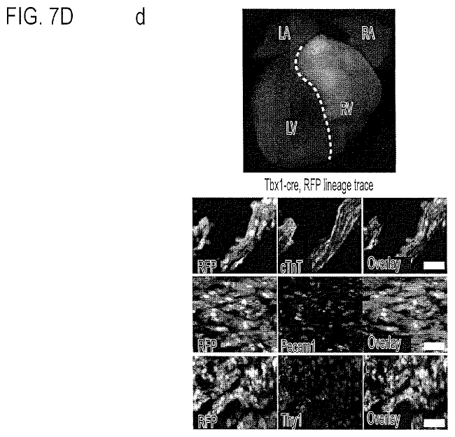

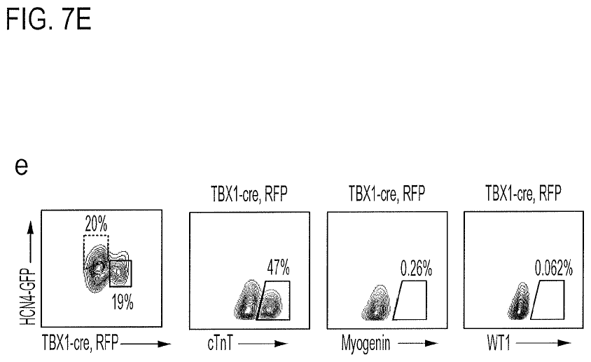

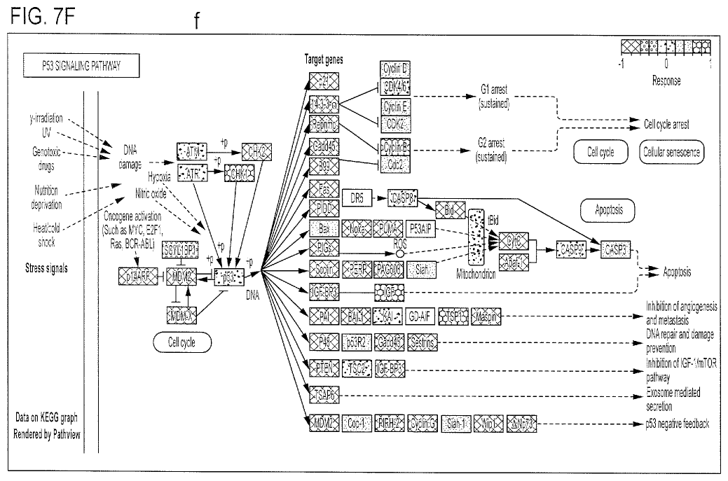

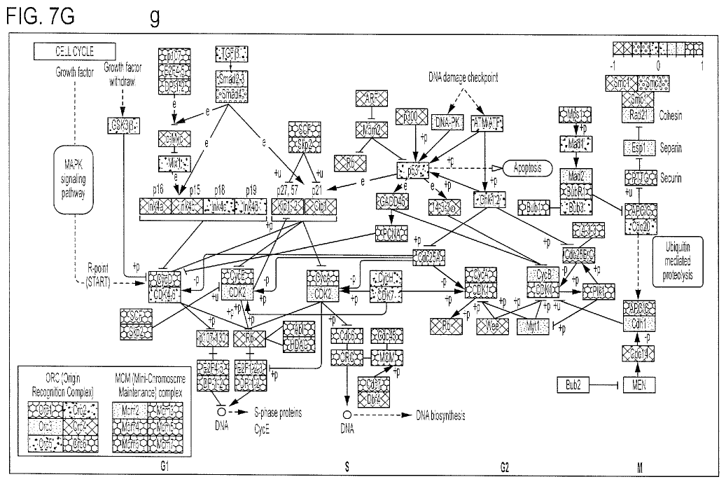

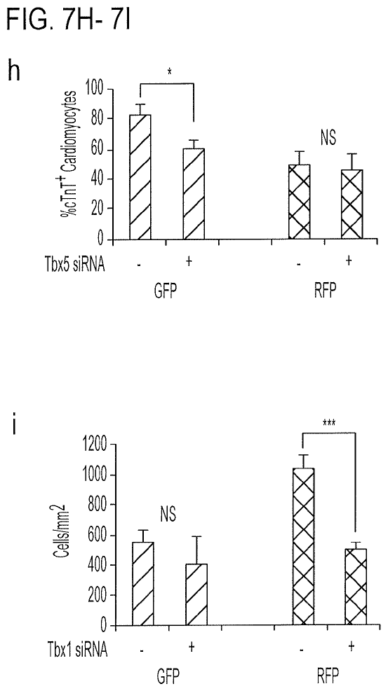

Next, the cellular identities of PSC-derived GFP⁺ and RFP⁻ cells was determined. To do this, GFP⁺ and RFP⁺ CPCs were FACS-isolated from the spheroids at day 5.5 or from Hcn4^{GFP}. Tbx1^{Cre}; Ai9 mouse embryos at E7.75 and subjected to RNA-sequencing. Genome-wide transcriptome analysis revealed a high correlation between in vivo and in vitro CPCs (GFP: in vitro vs. in vivo, R²=0.91, RFP: in vitro vs. in vivo, R²=0.98) (FIG. 7A), which indicated similar gene expression profiles between PSC-derived cells and their in vivo counterparts. Expression levels of Hcn4 and Tbx1 were also confirmed in the analyzed populations (FIG. 7B).

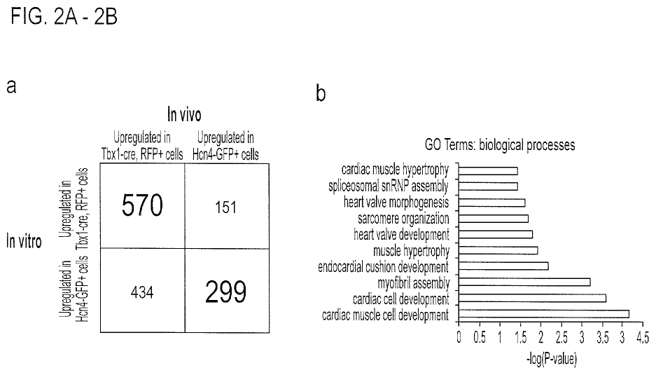

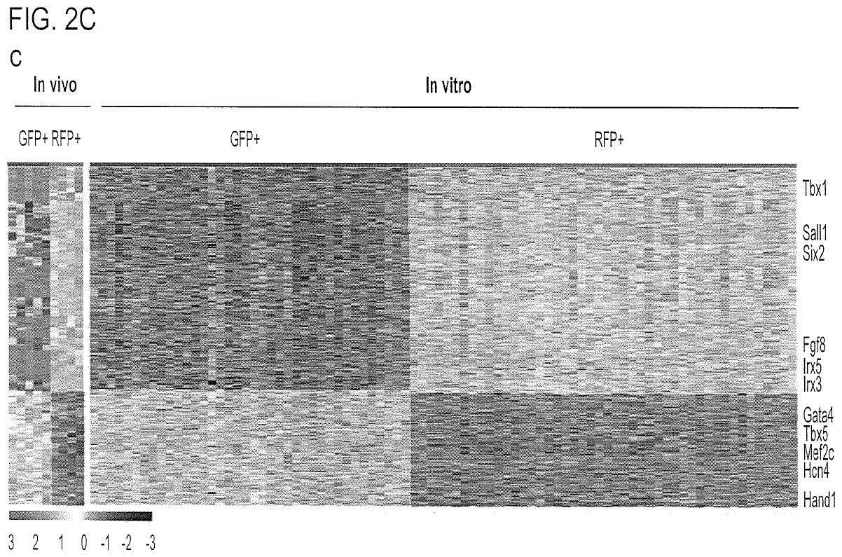

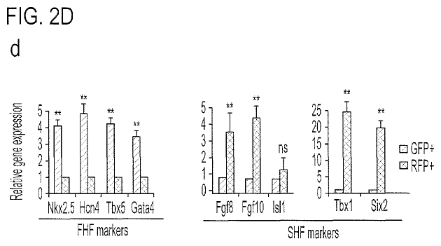

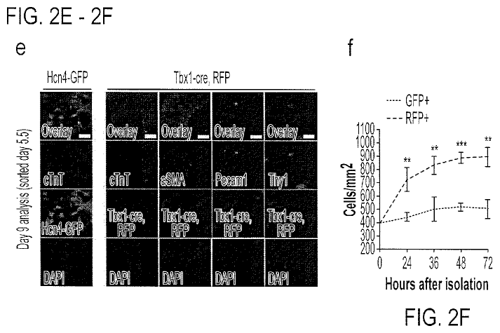

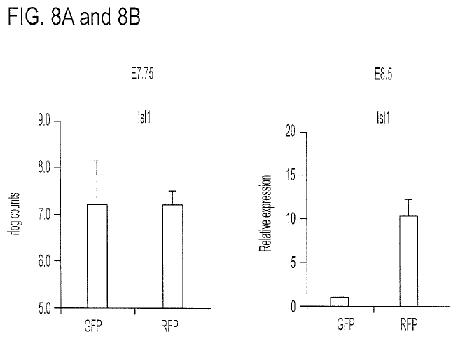

1,968 genes were identified that were differentially regulated between GFP⁺ and RFP⁺ cells in vivo (adjusted p-value<0.1); of these, 1,454 genes were differentially regulated between the GFP⁺ and RFP⁺ populations in vitro. Among these, 869 genes showed higher expression in the same population (i.e. GFP⁺ or RFP⁺) both in vitro and in vivo (FIG. 2A). Gene Ontology (GO) analysis for these genes showed enrichment for terms relevant to cardiovascular cellular and organ development (FIG. 2B). This gene list included known FHF genes (Gata4, Tbx5, Mef2c, Hand1) and SHF genes (Sal1, Six2, Fgf8, Irx3, Irx5) and could be used to distinguish GFP⁺ and RFP⁺ cells in vitro (FIG. 2C). 585 genes showed different expression patterns compared between in vitro and in vivo. GO analysis of these genes showed enrichment of terms such as ‘cell-substrate adhesion’ (FIG. 7C), which was likely due to differences between the in vivo and in vitro microenvironments. The enrichment of FHF and SHF genes in GFP⁺ and RFP⁺ cells was further confirmed by qPCR analysis (FIG. 2D). Isl1 was expressed in both cell types without significant difference in levels (FIG. 2D). This was consistent with the earlier finding (FIG. 6E-6H) and the previous reports that Isl1 was a pan-cardiac marker^{40, 41}. Its expression is however downregulated at E8.5 in GFP⁺ cells (FIGS. 8A and 8B), suggesting that Isl1 was transiently expressed in FHF. The prolonged expression of Isl1 in RFP⁺ cells may correlate with its role for SHF development^{21, 42}.

To verify the cardiomyogenic potential of PSC-derived GFP⁺/RFP⁺ cells, the cells were immediately isolated after appearance of GFP and RFP (day 5.5), when no cTnT⁺ cells were detected, and differentiated for 4 days. Consistent with the earlier FACS analysis (FIG. 1D), GFP⁺ cells robustly gave rise to cardiomyocytes (FIG. 2E), which suggested that they were committed to a cardiomyogenic lineage. In vivo, SHF progenitors proliferated prior to differentiation and gave rise to most cell types of the heart, including cardiomyocytes, the endothelium and fibroblasts (FIG. 7D)^{8, 20, 39, 43}. Similarly, RFP⁺ cells gave rise to cells positive for cTnT (cardiomyocytes), Pecam-1 (endothelia), α-SMA (smooth muscle) and Thy1 (fibroblasts) (FIG. 2E). As Tbx1 is also expressed in head muscle progenitors in developing embryos 44, the early muscle marker Myogenin was isolated in the spheroids at day 9. No meaningful percentages of RFP⁺ and Myogenin⁺ cells (0.26%) were detected compared to RFP⁺ and cTnT⁺ cells (47%) (FIG. 7E). In addition, cells positive for the epicardial marker Wilms tumor 1 (WT1) were nearly undetectable (0.062%) (FIG. 7E), indicating that RFP⁺ almost exclusively gave rise to cardiac lineages.

KEGG pathway analysis revealed increased cell cycle activity in RFP⁺ cells compared to GFP⁺ cells (FIG. 7F). GFP⁺ cells showed increased activity of the p53 signaling pathway, commonly known as a negative regulator of cell cycle activity (FIG. 7G). Consistently, RFP⁺ cells doubled in numbers within 36 h of culture, whereas GFP⁺ cells showed a modest level of proliferation (FIG. 2F). These indicated that RFP⁺ cells represented multipotent and proliferative CPCs analogous to the SHF in vivo. Since PSC-derived GFP⁺ and RFP⁺ represented distinct FHF and SHF CPCs, their potential was tested for heart field/chamber-specific disease modeling. To do this, Tbx5 was knocked down, the causative gene for Holt-Oram syndrome, which is associated with left-sided ventricular heart malformation including hypoplastic left heart syndrome in humans and mice^{45, 46}. Reduced levels of Tbx5 significantly decreased the number of cardiomyocytes formed from GFP⁺ cells but had no effect on RFP cells (FIG. 7H). Contrarily, knocking down Tbx1, a causative gene for DiGeorge syndrome associated with OT defects, negatively affected the proliferation of RFP⁺ cells, but not GFP⁺ cells (FIG. 7I). Together, these findings supported the recapitulation of the in vivo process and gene expression of GFP⁺ and RFP⁺ populations in the PSC spheroid system, and thus, this system may be used to model cellular and molecular heart field/chamber-specific events associated with CHDs.

### Example 3: FHF and SHF Progenitors were Specified Via the Bmp/Smad Pathway and a Smad-Independent Bmp/Wnt Pathway, Respectively, in PSC-Derived Spheroids

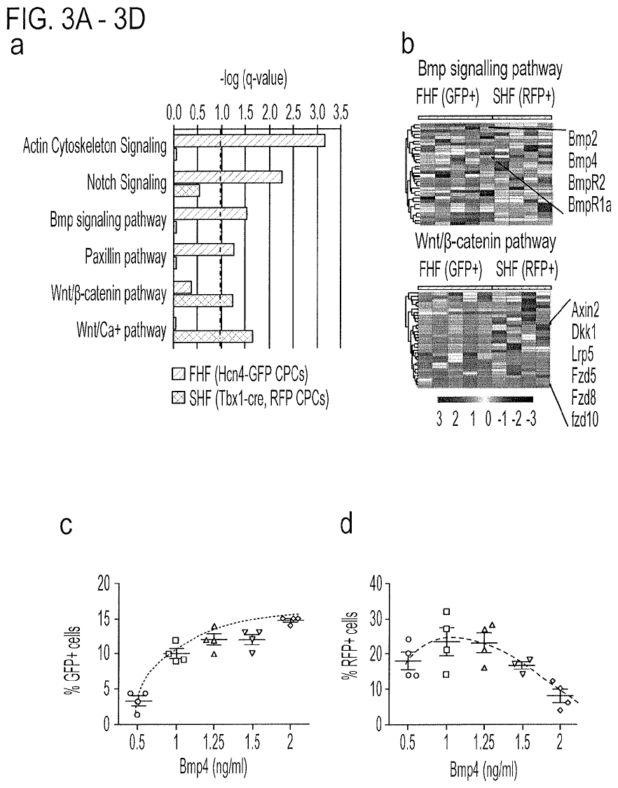

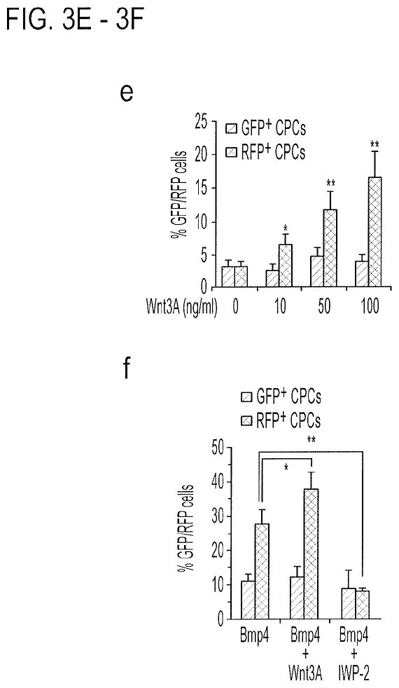

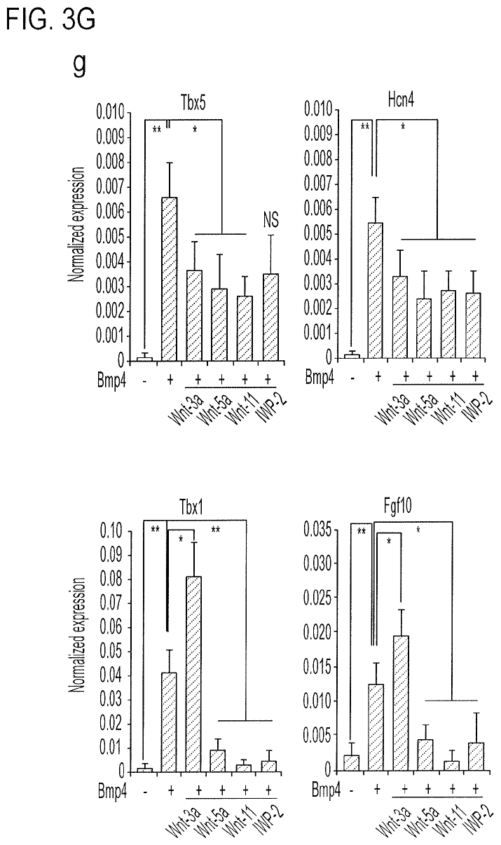

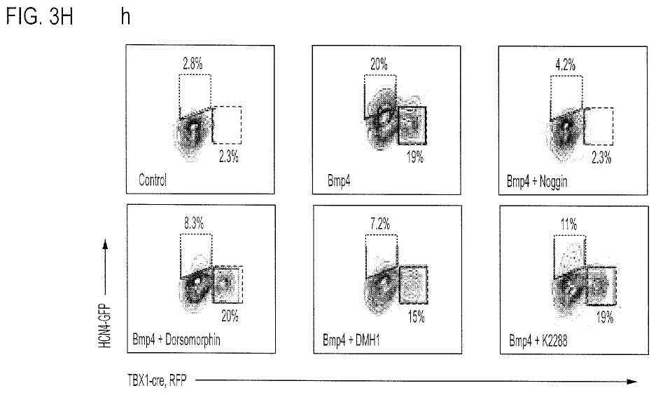

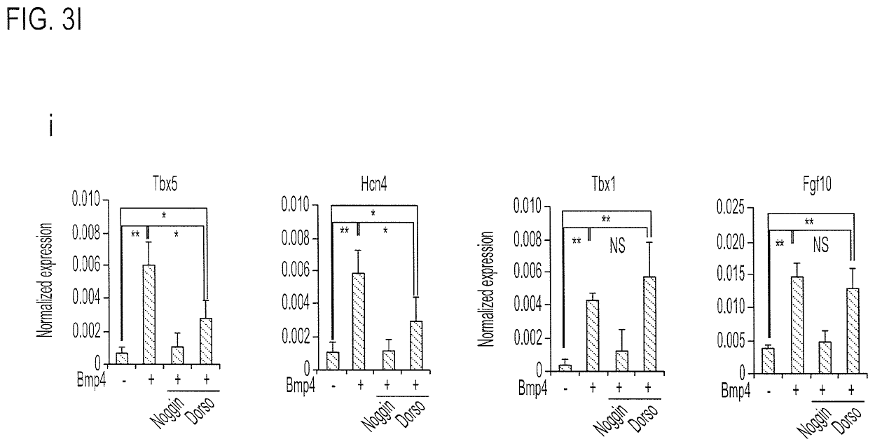

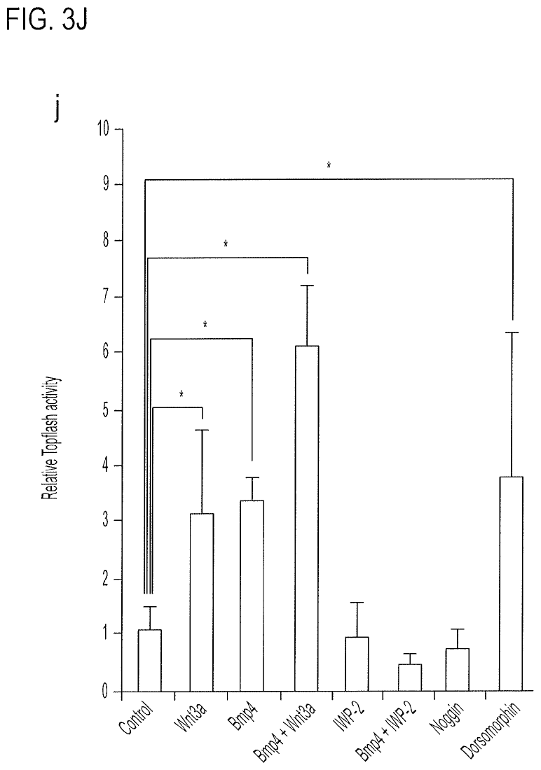

To gain mechanistic insights into inductive signals of heart fields, Ingenuity Pathway Analysis (IPA)^{47, 48} was performed on the lists of 592 and 1,377 genes that were differentially upregulated in the GFP⁺ and RFP⁺ cells, respectively. IPA utilizes an input gene list and a curated database of literature-derived pathways to infer which canonical pathways are most significant to the input data set. The analysis was focused on pathways related to ‘organism growth and development’. IPA inferred that activity of Actin cytoskeleton, Paxillin, Notch and Bmp signaling pathways are enriched in GFP⁺ cells while Wnt activity was enriched in RFP⁺ cells (FIG. 3A). The high activity of Actin cytoskeleton and Paxillin signaling pathways likely reflected the presence of structural genes in FHF cells. Notably, the key members of Bmp or Wnt/β-catenin signaling components—BmpR1a, Bmp2 and Bmp4 or Axin2, Fzd, and Dkk1—were upregulated in GFP⁺ or RFP⁺ cells, respectively (FIG. 3B). The effect of Bmp signals was evaluated during heart field specification. In order to minimize the possibility of influencing heart field cells after induction, all of the data were analyzed within 12 hs after the appearance of GFP⁺/RFP⁺ cells. Increasing Bmp4 levels promoted induction of both GFP⁺ cells and RFP⁺ cells, but only GFP⁺ cells responded in a dose-dependent manner (FIGS. 3C and 3D). RFP⁺ cells were also induced, but their induction was generally maintained except at the highest concentration (FIG. 3D).

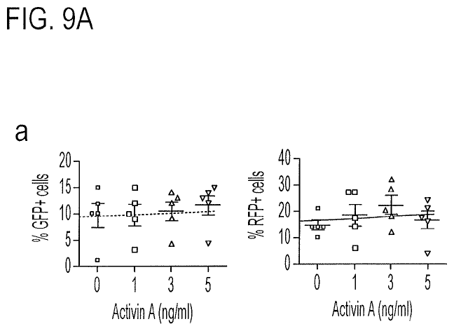

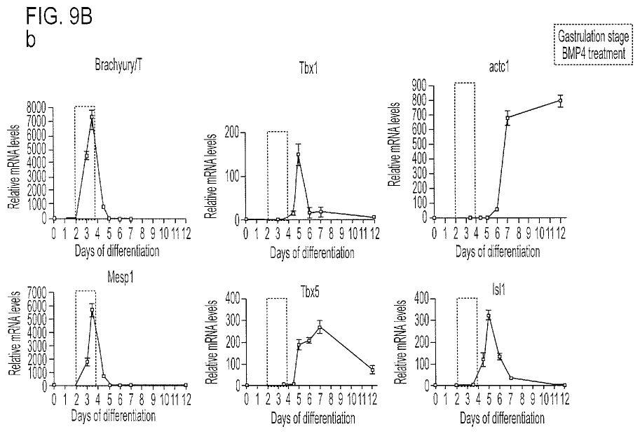

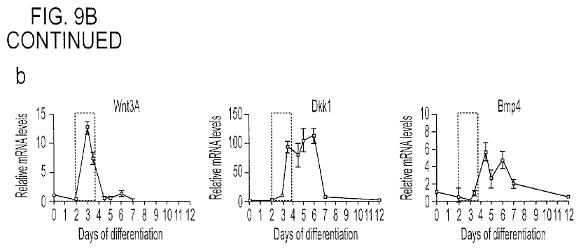

On the other hand, increasing levels of Activin A, a key ligand for Activin/TGF-0 signaling, had no apparent effect (FIG. 9A). This suggested that Bmp signaling promoted FHF specification and may allow SHF specification. Interestingly. increasing concentrations of Wnt3A correlated with increased numbers of RFP⁺ cells but did not affect GFP⁺ cells (FIG. 3E). This indicated that Wnt signaling specifically promoted specification of the SHF. Intriguingly, the observed Bmp4-mediated induction of RFP⁺ cells was abolished by the porcupine inhibitor IWP-2, a potent inhibitor of Wnt secretion (FIG. 3F). This suggested that Bmp signaling specified SHF via endogenous Wnt ligands.

To investigate the crosstalk between Bmp and Wnt signals, the spheroids were treated with Bmp4 and Wnts in combinations and analyzed expression levels of FHF (tbx5, hcn4) and SHF (tbx1, fgf10) markers. Similar to the earlier finding, Bmp4 alone increased expression of both heart field markers, but SHF marker expression was suppressed, when IWP-2 was added (FIG. 3G). Likewise, the addition of Wnt3A resulted in a further increase of the SHF markers and a reduction of the FHF markers (FIG. 3G).

The combination with Wnt5A or Wnt11 caused an overall reduction of all markers (FIG. 3G), indicating that noncanonical Wnts signaling did not regulate heart field specification. These data suggested that Bmp signaling increased canonical Wnt signaling for SHF specification. To test this, canonical Wnt activity was measured with its readout Topflash. Indeed, treatment with Bmp4 alone increased topflash activity, and the activity was further increased when the cells were treated in combination of Bmp4 and Wnt3A (FIG. 3J).

Based on the finding that Bmp signals promote both heart field specification and Wnt activity, it was tested whether Bmp signals were necessary for these events, done by treating the spheroids with Noggin, which blocks Bmps from binding their receptors⁴⁹. The treatment abolished Bmp's inductive effects on GFP⁺/RFP⁺ cells, accompanied with markedly reduced Wnt activity (FIG. 3H). Since Bmp-mediated induction of RFP⁺ cells required Wnt signaling, these data suggested that Bmp signaling was required and sufficient for specifying both FHF and SHF cells and activated Wnt signaling for the SHF specification. Notably, dorsomorphin, DMH1, and K2288, selective Bmp type I receptor inhibitors of SMAD-dependent signaling^{50, 51} suppressed GFP⁺ cell induction and FHF genes without significantly affecting RFP⁺ cells, SHF genes, and Wnt activity (FIG. 3H-3J).

This was further supported by the co-treatment of cardiac spheroids with Noggin or dorsomorphin, which showed inhibition or no effect, respectively, on the Bmp-mediated increase in topflash activity. Together, these data suggested that FHF cells were specified through the BMP/SMAD pathway, whereas SHF cells were specified via a SMAD-independent BMP/Wnt pathway.

### Example 4: Cxcr4 Identified SHF Progenitors In Vivo and In Vitro

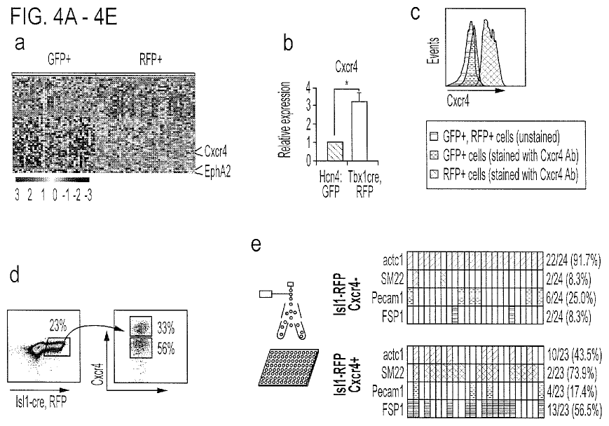

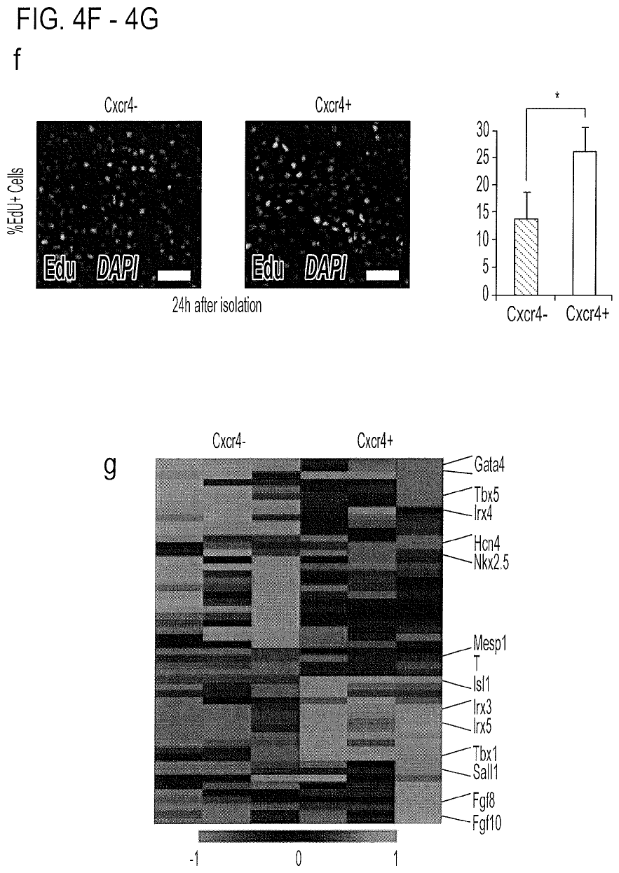

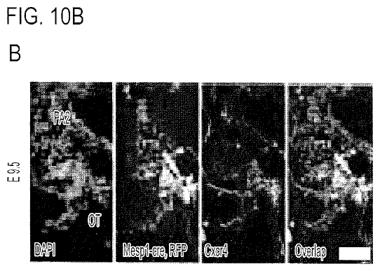

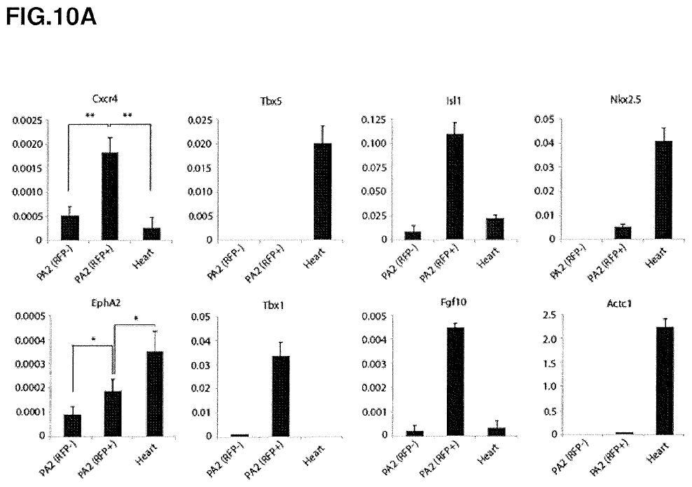

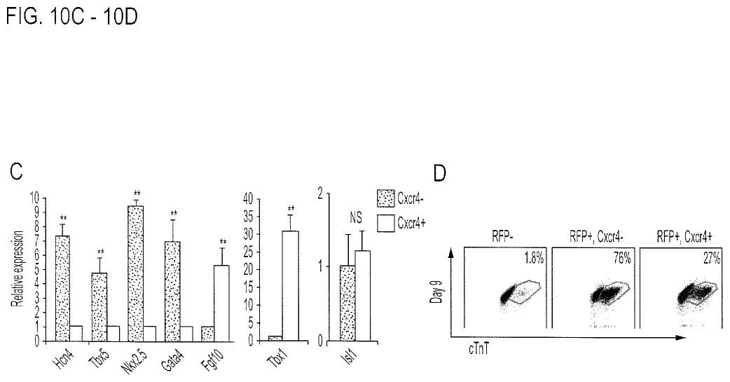

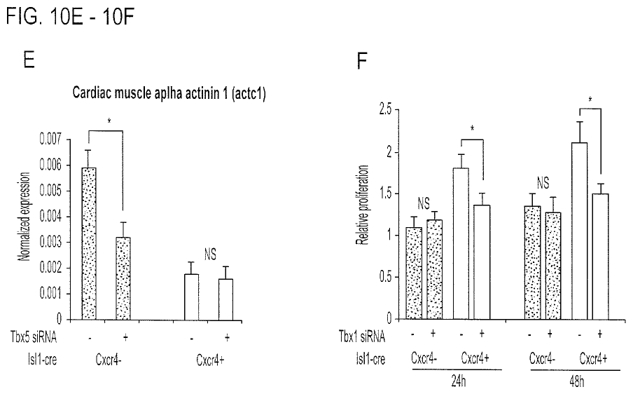

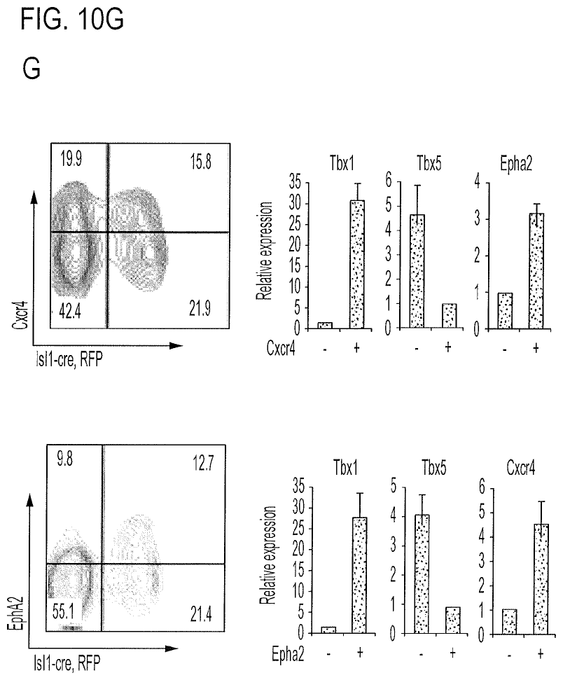

Developing a non-genetic way to identify and isolate specific cell types is crucial for PSC-based regenerative medicine⁵². Therefore cell surface markers were searched for and enriched in FHF or SHF cells. By RNA-sequencing analysis, 240 differentially expressed surface receptors between GFP⁺ and RFP⁺ cells (FIG. 4A) were identified. Given that SHF cells were migratory^{43, 51}, genes involved in cell mobilization were focused on and the two receptors C-X-C Chemokine Receptor type 4 (Cxcr4) and Ephrin type-A receptor 2 (EphA2) were identified, which were both upregulated in the RFP⁺ cells compared to GFP⁺ cells in vitro. Their differential expression was confirmed by qPCR in vivo and in vitro (FIG. 4A and FIG. 10A).

In order to determine the expression in vivo, the expression of Cxcr4 and Epha2 was analyzed along with other cardiac markers in the Mesp1-derived progeny in the mesodermal core of the 2^{nd} pharyngeal arch at E9.0, which harbors undifferentiated and expansive SHF-CPCs⁴³. To do this, arches were dissociated from Mesp1^{Cre}; Ai9 mice and isolated RFP⁺ and RFP⁻ cells by FACS followed by qPCR analysis. Both Cxcr4 and Epha2 were significantly enriched in RFP⁺ CPCs compared to RFP⁻ cells (FIG. 10A). While Epha2 levels were increased in the developing heart, the Cxcr4 expression pattern was similar to that of undifferentiated CPC markers (Tbx1, Fgf10, Isl1), indicating that Cxcr4 exclusively marked undifferentiated SHF-CPCs (FIG. 10A).

The co-expression of Isl1 and Cxcr4 was further confirmed in the mesodermal core of PA2 by immunohistochemistry (FIG. 10B). Additionally, FACS analyses of GFP⁺/RFP⁺ CPCs at day 5.5 confirmed that Cxcr4 exclusively marked Tbx1-Cre, RFP⁺ but not Hcn4-GFP⁺ CPCs in cardiac spheroids (FIG. 4C).

To determine whether Cxcr4 marked SHF-CPCs in vitro, an ESC line was generate from Isl1^{Cre}; Ai9; MHC^{GFP} mice in which RFP permanently marks Isl1 progeny and cardiomyocytes can be identified by GFP expression 54. After 5.5 days of differentiation, Cxcr4 identified a subset of RFP⁺ CPCs (FIG. 4D and FIG. 10D). RFP⁺/Cxcr4⁻ or Cxcr4⁺ cells were FACS-isolated with Cxcr4 antibody and analyzed with qPCR. Accordingly, the FILF markers Rcn4, Tbx5, Nkx2.5, and Gata4 were enriched in Isl1-Cre, RFP⁺/Cxcr4⁻ CPCs, whereas the SHF markers Fgf10 and Tbx1 were enriched in RFP⁺/Cxcr4⁺ cells (FIG. 10C). Isl1 levels were not significantly different between Cxcr4⁺ and Cxcr4⁻ CPCs, similar to the expression levels in Hcn4-GFP⁺, Tbx1-Cre, RFP⁺ CPCs (FIG. 10C). In order to determine the cardiac differentiation potential of the two populations, single RFP⁺/Cxcr4⁻ or Cxcr4⁺ cells were isolated from day 5.5 spheroids and clonally expanded for 7 days. RFP⁺/Cxcr4⁻ cells primarily differentiated into cardiomyocytes, while RFP⁺/Cxcr4⁺ cells gave rise to multiple cardiac lineages (FIG. 4E). RFP⁺/Cxcr4⁺ cells were more proliferative than RFP⁺/Cxcr4⁻ cells, determined by nucleoside 5-ethynyl-2′-deoxyuridine (EdU) incorporation (27% vs. 14%) (FIG. 4F). The cellular identities of Cxcr4⁺ or Cxcr4⁻ cells were further confirmed by microarray analysis (FIG. 4G). These data suggest that PSC-derived FHF or SHF cells can be distinguished and purified based on their expression of Cxcr4. By qPCR it was confirmed that Epha2 levels were elevated in Cxcr4⁺ CPCs. Likewise, FACS analyses demonstrated that Epha2 marked a subset of RFP⁺ CPCs similar to Cxcr4. Accordingly, Cxcr4 levels were increased in RFP⁺, Epha2⁺ cells while Tbx1 and Tbx5 showed a similar expression pattern to that of RFP⁺, Cxcr4⁺ CPCs, implying that Cxcr4 and Epha2 marks the same population of SHF CPCs.

Finally, the cardiac disease modeling potential was validated in Isl1-Cre, RFP⁺, Cxcr4^{+/-} CPC populations by knocking down Tbx5 and Tbx1. Importantly, Tbx5 knockdown only affected cardiogenesis in Cxcr4⁻ CPCs (FIG. 10E), whereas Tbx1 knockdown only had an effect on Cxcr4⁺ CPCs (FIG. 10F), similar to the knockdown experiments in Hcn4-GFP⁺ and Tbx1-Cre, RFP⁺ CPCs (FIGS. 7H and 7I).

Taken together, these results demonstrated how Cxcr4 and Epha2 expression identified undifferentiated SHF-CPCs, and how Cxcr4 and EphA2 may be used to develop non-genetic approaches to isolate undifferentiated CPCs from mouse PSC cultures.

### Example 5: CXCR4 Identified SHF Progenitors in Human iPSC Spheroids

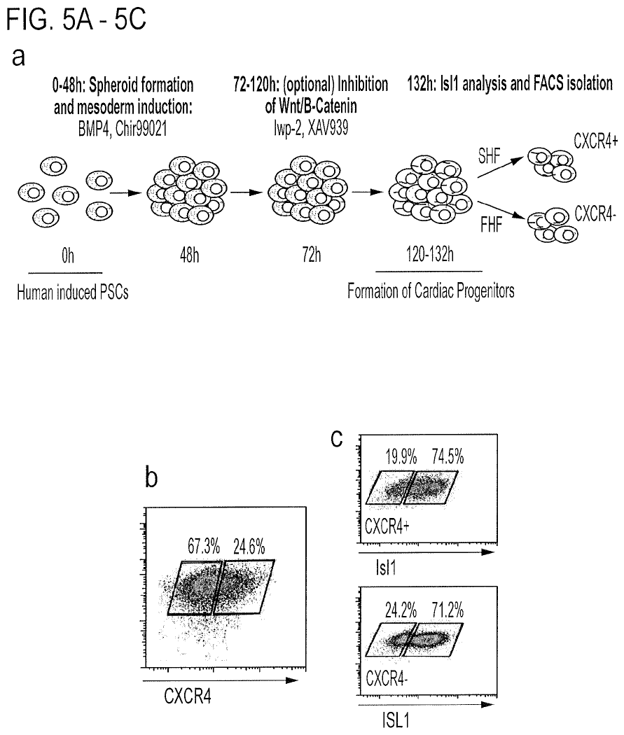

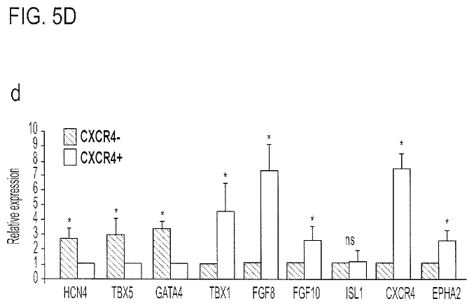

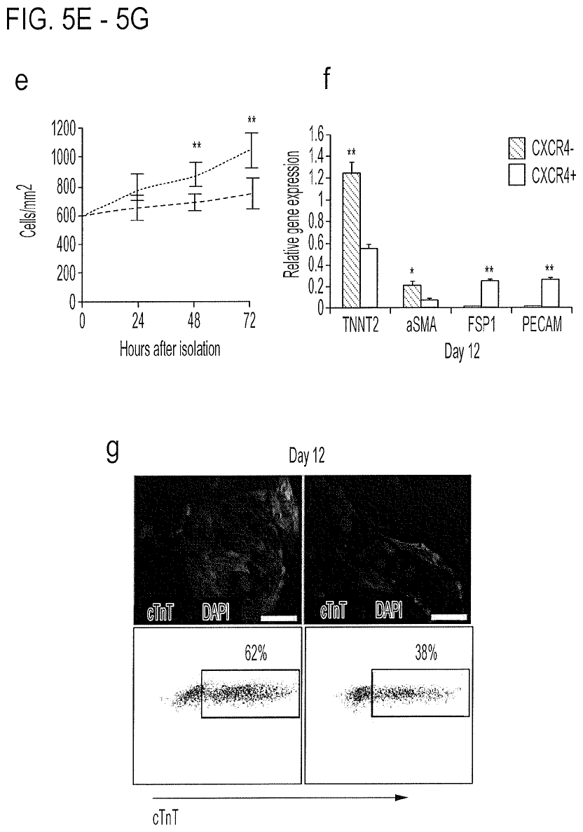

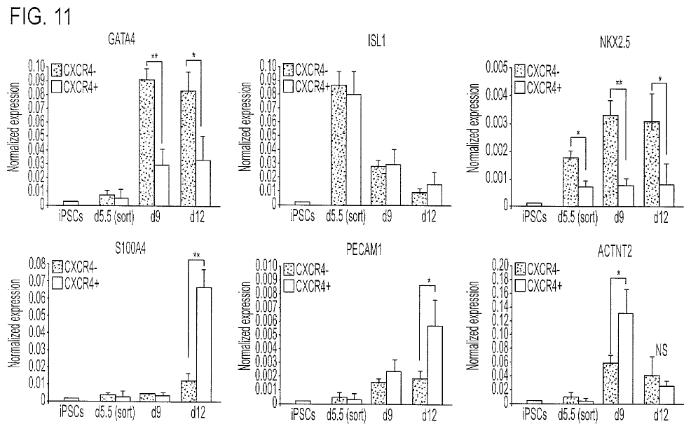

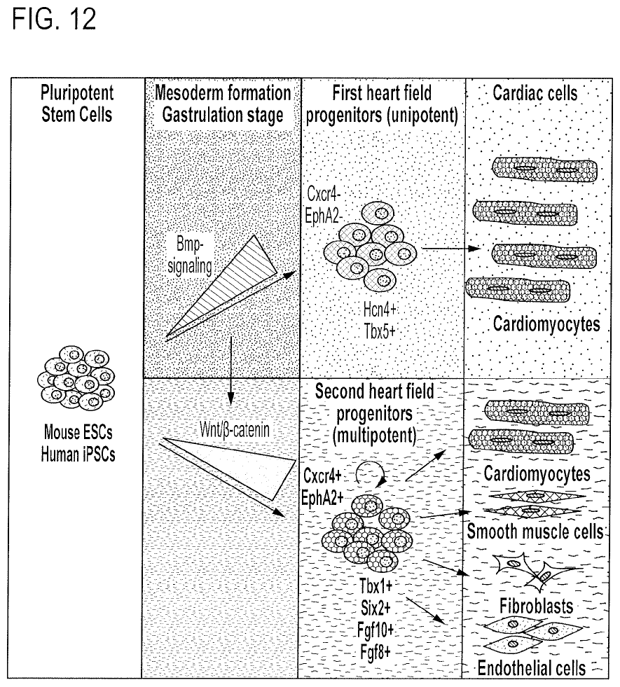

To determine whether two heart fields were induced in human PSCs, a protocol was devised for hiPSCs to generate spheroids based on Bmp4 and Wnt activation with the small molecule inhibitor Chir99021 that allowed inducing high percentages of ISL1 CPCs⁵⁵ (FIG. 5A). At day 5.5, CXCR4⁻ and CXCR4⁺ cells were isolated from the spheroids by FACS and approximately 75-80% of cells in both populations expressed the CPC marker ISL1, indicating a commitment to the cardiac lineage (FIG. 5B-5C). In addition, the expression of heart field genes in the sorted CPCs was analyzed. Similar to the mouse PSC system, the FHF genes (HCN4, TBX5, GATA4) or the SHF genes (TBX1, FGF10, FGF8) were highly upregulated in CXCR4 cells or CXCR4⁺ cells, respectively (FIG. 5D). The CXCR4⁺ cells were more proliferative than CXCR4⁻ cells while CXCR4⁻ cells were more cardiomyogenic than CXCR4⁺ cells (62% Vs 38%) (FIG. 5E-5G). After differentiation, CXCR4⁺ cell progeny expressed high levels of smooth muscle, endothelial, and fibroblast markers (FIG. 5F and FIG. 11), supporting their multipotency, while cells derived from CXCR4⁻ CPCs expressed high levels of cardiomyocyte markers and low levels of endothelial/fibroblast markers (FIG. 5F). These data suggested that FHF and SHF cells were generated and distinguished by CXCR4 expression in human iPSC spheroids.

### Discussion

In the current study, a mouse and human PSCs to model the earliest stages of heart field development was used, with the goal to identify the inductive signals of the two heart field and to create a model system that allows the study of heart field-specific developmental events. The derivation of embryonic stem cells from developing Hcn4-GFP, Tbx1-Cre, Ai9 embryos allowed direct comparison of CPCs between in vivo and in vitro, and thereby use mouse embryos as reference for heart field specification in vitro. In particular, the use of the Tbx1-Cre allele allowed tracing and followed RFP⁺ progeny in the spheroid system. While an earlier study used a two-reporter system with Mef2c/Nkx2.5 enhancer-driven RFP/GFP, the analysis was done at a later stage (E9.5), when the heart is present⁵⁶. The findings from this work provide a scheme of which distinct heart field populations are specified during gastrulation by gradients of Bmp and Wnt/β-catenin signaling and can be identified by based on Cxcr4 expression (FIG. 6).

Herein, it is proposed that the FHF is induced by Bmp/Smad signaling during gastrulation stage, whereas the SHF is induced by Bmp-mediated activation of canonical Wnt signaling. Collectively, these new insights are expected to provide a framework for studying the earliest stages of mammalian cardiac development and a platform for efficient generation of chamber-specific progenitors for human iPSC-based heart disease modeling.

The findings that the LIM homeodomain transcription factor Isl1 progeny give rise to the entire heart is supported by several earlier studies^{37, 40, 41, 57} Isl1 has been regarded a SHF marker since it was first described in a fate-mapping study with Isl1-IRES-Cre mice, where Cre was inserted into the exon encoding a LIM domain^{21, 42}, and since Isl1-null embryos primarily affect development the OFT and RV 2, indicating that Isl1 plays an essential role in development of the SHF and structures derived thereof. However, retrospective lineage tracing experiments using an efficient Isl1-Cre knock-in mouse line showed that most cells in the LV also originate from Isl1-expressing cells³⁷.

Other studies have reported that Isl1 protein is expressed at E7.5 throughout the anterior intra-embryonic coelomic walls and proximal head mesenchyme, regions that encompass both the FHF and the SHF in mouse⁴⁰, and more recently, that Isl1 is expressed in Tbx5-expressing cells isolated from the cardiac crescent⁴¹, implying that Isl1 may be temporarily expressed in both heart fields. It has been suggested that the inefficient recombination activity of the original Isl1-IRES-Cre might have contributed to the conclusion made early⁵⁸. In the present study whole mount in situ hybridizations were performed to demonstrate that Isl1 is indeed expressed in the primitive heart tube of E8.0 mouse embryos. Based on the studies herein, it was concluded that Isl1 was a pan-cardiac marker, expressed in all undifferentiated CPCs similar to the transcription factor Sall1³³.

Based on the observations and the FACS analyses, GFP⁺ and RFP⁺ cells appear invariably around the same time. Specific cases have not been observed when one reporter appears first. However, there are several developmental and technical considerations that make it difficult to conclude the order of their induction: First, while both of the FHF and the SHF appear at the cardiac crescent stage (E7.25-7.75), the work herein and published studies suggest that their precursors might be specified during gastrulation (E6.5-7.0) 7, 8. Second, there might be a slight delay as RFP expression is activated upon Tbx1-Cre expression. Finally, Hcn4-GFP is a fusion protein emitting signals lower than RFP.

The concept that both heart fields are specified in nascent mesoderm is supported by two studies^{7, 8} where FHF and SHF progenitors were shown to be present in two temporal pools of Mesp1-expressing cells during gastrulation. Although the fluorescent reporters used to visualize the two heart fields were not activated during germ layer formation, the findings from the precardiac spheroid system clearly demonstrated that their specification was positively regulated by Bmp and Wnt signals during a gastrulation stage, which was defined by a temporal expression of Brachyury and Mesp1 (FIG. 9B)^{5, 7, 8, 11}. It will be of great importance to determine the specific inductive roles of the two morphogens in heart field formation in vivo.

Curiously, increasing levels of Activin A did not have a significant effect on cardiogenesis and no overall effect on heart field induction. This may suggest a broader role of Activin A in mesoderm formation. This is supported by the previous report that signaling from the Activin A receptor Acvr1b regulated the fates of mesendoderm progenitors¹³. In fact, Acvr1b signaling was shown to favor endoderm formation by repressing expression of members of the Id family of DNA-binding protein inhibitors, whereas its reduction depresses Id genes and promotes cardiac mesoderm formation¹³.

Bmp signaling directly activated transcription of Id1⁵⁹⁻⁶¹, which is necessary and sufficient to induce cardiac differentiation in mouse and human PSCs via upregulation of FHF genes, but not SHF genes¹³. In addition, mice deficient of Id1-4 fail to express the FHF genes Smarcd3, Tbx5, and Nkx2.5 in the anterior region of the cardiac crescent¹³, suggesting that Bmp signaling may activate the FHF program through Id genes. Consistently, the RNA-sequencing analysis revealed that Id1, 2 and 4 were upregulated in Hcn4-GFP⁺ CPCs at day 5.5. Activin A and Bmp4 were shown to play a pivotal role in generating distinct subpopulations of mesoderm in a human PSC system⁶². They are distinguished by expression of RALDH2 and CD235a/CYP26A1 and give rise to atrial and ventricular cardiomyocytes, respectively⁶². The specification of ventricular progenitors was dependent on a higher ratio of Activin A to Bmp4 signaling than one required for the atrial lineage⁶².

Herein, it was found that ALDH1A2 (RALDH2) was highly expressed in SHF CPCs both in vivo and in vitro. This may suggest that SHF progenitors contain RALDH2⁺ atrial progenitors. The finding that Bmp4-mediated upregulation of canonical Wnt signaling was necessary for specification of multipotent cardiac progenitors provides new insights into how the distinct heart fields are specified. In vivo, Bmp4 and Wnt/β-catenin signaling played critical roles in early cardiogenesis^{63, 64}. However, it remains unclear which cell types secrete Bmp and Wnt ligands and how these signals influence early heart field development. The findings suggest that the Bmp4-receiving cells, giving rise to the FHF, may play an inductive role for SHF specification via positive regulation of expression of Wnt ligands. Evidence presented here show the presence of distinct pathways regulating these events, a.

The ability to recapitulate and monitor heart field development in a PSC system has enabled investigation into the molecular pathways that regulate early cardiac fate decisions. The findings emphasize the importance of the PSC system in understanding the earliest stages of cardiac development. In fact, the system offers many advantages, such as an unlimited source to generate mesodermal cells, cell differentiation in a defined condition, and time-lapse capability, and can avoid the experimental difficulties associated with gastrulation-stage embryos such as size, staging, and quantity. While expression trend patterns between FHF and SHF corresponded very well between in vitro and in vivo, absolute expression values (for example, normalized counts) did not correspond well between in vivo and in vitro. This phenomenon is not unique to the study but rather observed frequently in in vitro, stem cell-derived tissue models⁶⁵. It will be important to investigate how the values are differentially regulated in vitro. There are several heart field/chamber-specific CHDs including hypoplastic left heart syndrome and hypoplastic right heart syndrome^{29, 66} as well as some chamber-specific cardiomyopathies and tachyarrhythmias like arrhythmogenic right ventricular cardiomyopathy or right ventricular outflow track ventricular tachycardia^{31, 67} The pathogenesis of these diseases has remained unexplored to a significant extent, partly due to the inability to obtain cardiac tissue from patients. Thus, the method offers a unique opportunity to study heart field/chamber-specific cardiac diseases using patient derived transgene-free CPCs.

### Materials and Methods

Generation of Hcn4-GFP, Tbx1-Cre, Ai9 mice and ESCs

Hcn4-GFP, Tbx1C, Ai9 mice were obtained by crossing Hcn4-GFP mice³⁸ with Tbx1-Cre mice¹⁷ and Ai9 reporter mice (stock no. 007909, Jackson Laboratory). The appearance of the vaginal plug was considered as day 0.5 of gestation (E0.5). Mouse ESCsHcn4-GFP; Tbx1-Cre; Ai9 were derived from blastocysts (E3.5) harboring Hcn4-GFP; Tbx1-Cre; Ai9 and mESCIsl1-Cre, αMHC-GFP-GFP; Ai9, were derived from blastocysts (E3.5) harboring Isl1-Cre⁴², αMHC-GFP⁵⁴; Ai9. All animals were housed at the Johns Hopkins Medical Institutions. All protocols involving animals followed U.S. NIH guidelines and were approved by the animal and care use committee of the Johns Hopkins Medical Institutions.

Cell Work

Mouse ESCs and human iPSCs were maintained and differentiated as previously described^{5, 43, 68} Briefly, mESCs were maintained on gelatin-coated dishes in maintenance medium (Glasgow minimum essential medium supplemented with 10% fetal bovine serum and 3 M Chir99021 and 1 μM PD98059 or 1000 U/ml ESGRO (Millipore, Billerica, Mass.), Glutamax, sodium pyruvate, MEM non-essential amino acids (Thermo Fisher Scientific). For spheroid formation and differentiation, mouse ESCs were plated in IMDM/Ham's F12 (Cellgro) (3:1) supplemented with N2, B27, penicillin/streptomycin, 2 mM GlutaMAX, 0.05% BSA, 5 ng/ml L-ascorbic acid (Sigma-Aldrich) and α-monothioglycerol (MTG; Sigma-Aldrich) at a final density of 100,000 cells/ml to allow spheroid formation. After 48 h spheroids were collected and transferred to ultra-low attachment plastic surface and induced for 40 hours with Activin A, Bmp4, Wnt3A, Wnt5A, Wnt11 (R&D Systems) alone or in combination. Human iPSCs were maintained in Geltrex-coated T25 flasks using Essential 8 medium. For spheroid formation and differentiation, hiPSCs were plated in RPMI plus B27 minus insulin with Bmp4 and CHIR99021 and incubated for 48 h. After 48 h media was changed to RPMI plus B27 minus insulin. The hiPSC line used in this study was developed by Dr. Linzhao Cheng, the Johns Hopkins University, and had was generated from a de-identified patient using an approved IRB protocol.

siRNA, Transfection and Luciferase Assays

For Tbx1 and Tbx5 knockdown experiments, Tbx1 and Tbx5 ON-TARGETplus SMARTpool siRNA or scrambled siRNA (Dharmacon/Thermo Fisher Scientic) was used at 5 nM for cell transfection. Cells were transfected with Lipofectamine LTX (Life Technologies) in single-cell suspensions. For TOP-flash luciferase assays, mESCs were transfected with Topflash constructs and Renilla constructs and analyzed as previously described 5.

Live Cell Imaging, EdU Labeling, Immunohistochemistry, and Microscopy

For live imaging, single cardiac organoids were plated in round bottom ultra-low plates (Cat #7007, Corning, Inc). Each well was imaged every hour for GFP and RFP expression up to 96 h using a BZ-9000 Fluorescence Microscope (Keyence). For EdU analysis, Click-it EdU kit (Life Technologies) was used followed by immunostaining with primary and secondary antibodies. For whole mount staining, embryos were fixed in 4% paraformaldehyde overnight and then 30% sucrose and then incubated with primary and secondary antibodies. For immunohistochemistry, embryos were fixed in 4% paraformaldehyde overnight and then 30% sucrose, and then embedded in OCT, sectioned and stained using standard protocols. Antibodies used were: mouse α-Islet1 (1:200; Cat. 39.3F7 Developmental Studies Hybridoma Bank, Iowa City, Iowa), rat α-RFP (1:200; Cat. 5F8 Chromotek), chicken GFP (1:500; Cat A10262 Invitrogen), rabbit Cxcr4 (1:500; Cat. 119-15995 Biotrend), rabbit aSMA (1:200; Cat. Ab5694 Abcam), Pecam-1 (1:100; Cat. 553371 BD Biosciences), Thy1 (Cat. 17-0902-82 eBiosciences), mouse cardiac TnT (1:500; Cat. MS-295-P1Thermo Fisher). Alexa Fluor secondary antibodies (1:500; Life Technologies) were used for secondary detection and images were acquired with an Evos fl microscope.

Flow Cytometry and Cell Sorting

Mouse embryos (E7.75) were dissected using forceps under a stereomicroscope (Zeiss) and regions of interest were dissociated and harvested using TrypLE. Embryoid bodies (EBs) and cells were dissociated and harvested using TrypLE. Single-cells were analyzed for RFP/GFP expression or sorted using a SH800 Cell sorter (Sony Biotechnologies). Live cells were analyzed for RFP and GFP expression and stained with antibodies targeting for the presence of appropriate markers. Cells were stained with the following antibodies: anti-mouse Cxcr4 conjugated with PerCP-eFluor 710 (1:200; 46-9991-80 eBiosciences) anti-mouse EphA2 conjugated with APC (1:100; Cat. FAB639A R&D systems), anti-human Cxcr4 conjugated with PE or APC (1:25; Cat. FAB170P R&D systems). For cTNT and Isl1 expression, cells were fixed with 4% paraformaldehyde (PFA) for 10 min, permeabilized with saponin (Sigma), stained with either mouse cTNT (1:500, Cat. MS-295-P1 Thermo Scientific) or mouse Islet1 antibody (1:200, Cat. 39.3F7 Developmental Studies Hybridoma Bank, Iowa City, Iowa), followed by incubation with secondary antibody conjugated with Alexa Fluor 647 (1:500, Invitrogen). Data was analyzed using FlowJo software.

Quantitative RT-PCR

RNA isolation was performed using either RNeasy Micro Kit (Cat #74004, Qiagen) or ARCTURUS® PicoPure® RNA Isolation Kit following the manufacturer's instructions, and cDNA was generated using the high-capacity cDNA reverse transcription kit (Applied Biosystems). qPCR reactions were performed using the Taqman (Applied Biosystems) or Sybr Select qPCR mix (Thermo Fisher) with indicated primers. Gene expression levels were normalized to Gapdh. For the clonal cell-fate analysis, single Isl1-Cre RFP+, Cxcr4− and Isl1-Cre RFP⁺, Cxcr4⁺ cells were sorted at day 5.5 into 384-well plates and allowed to grow and differentiate for 7 days. Appearance of colonies was visually confirmed by microscopy. RNA was isolated from 24 wells with colonies from Cxcr4− and Cxcr4+ sorted cells, respectively. Ct values<30 were considered positive. All samples were also analyzed for gapdh to exclude false-positive results.

Whole Mount In Situ Hybridization

Whole-mount in situ hybridization was performed as described previously⁶⁹, with designated antisense probes. Isl1 probe was prepared as described and HCN4 antisense probe was synthesized and purified after cloning Hcn4 cDNA into pBluescript II KS (see HCN4 primers). The probes were labeled with digoxigenin and anti-digoxigenin antibodies conjugated with alkaline phosphatase (anti-585 digoxigenin-AP, Roche) used for probe detection. Staining reactions were performed after washing with NTMT and incubation with BM-Purple (Sigma-Aldrich).

Library Preparation and Sequencing

GFP+ and RFP+ cells were isolated using a SH800 cell sorter (Sony Biotechnologies) into 96 plates containing water (2.4 mL) with RNase-free DNase I (0.2 mL; NEB) and RNase inhibitor (0.25 mL; NEB). Each sample represents 10 cells. DNase I was inactivated by increasing the temperature (72 C for 3 min), and samples were then stored on ice. Custom-designed 2A oligo 1-mL primer (12 mM, Integrated DNA Technologies^{26, 70} was added and annealed to the polyadenylated RNA by undergoing a temperature increase (72 C for 2 min) and being quenched on ice. A mixture of 1 mL of SMARTscribe reverse transcriptase (Clontech Laboratories), 1 mL of custom-designed TS oligo (12 mM, Integrated DNA Technologies 7°, 0.3 mL of MgCl2 (200 mM, Sigma), 0.5 mL of RNase inhibitor (Neb), 1 mL of dNTP (10 mM each, Thermo), and 0.25 mL DTT (100 mM, Invitrogen) were incubated at 42 C for 90 min, which was followed by enzyme inactivation at 70 C for 10 min. A mixture of 29 mL of water, 5 mL of Advantage2 taq polymerase buffer, 2 mL of dNTP (10 mM each, Thermo), 2 mL of custom-designed PCR primer (12 mM, Integrated DNA Technologies⁷⁰, and 2 mL of Advantage2 taq polymerase was directly added to the reverse transcription product, and the amplification was performed for 19 cycles. The amplification product was purified using Ampure XP beads (Beckman-Coulter). Libraries and transposome assembly were made using a previously published protocol⁷¹. Briefly, 100 pg of total cDNA was added to a 2× tagment DNA Buffer (TD) (2×TAPS buffer: 20 mM TAPS-NaOH, 10 mM MgCl₂ (pH 8.5) at 25 C, and 16% weight volume (w/v) PEG 8000), and then spiked with 0.5 mL of 1:64 diluted Tn5 (Epicenter) and incubated for 8 min at 55 C. Tn5 was stripped off from the tagmented DNA by adding 0.2% SDS for a final concentration of 0.05%.

Libraries were enriched used KAPAHiFi, which included 5× Kappa Fidelity Buffer, 10 mM dNTPs, and HIFI polymerase, and 1 uL of index primers was used directly in the enrichment PCR amplification of libraries for the Illumina sequencers for a 50-mL reaction. The PCR program was as follows: 5 min at 72 C and 1 min at 95 C, and then 16 cycles at 30 s at 95 C, 30 s at 55 C, 30 s at 72, and 5 min at 72. For analysis, raw sequencing reads were trimmed using Trimmomatic(0.36) with a minimum quality threshold of 35 and minimum length of 36 (Bolger, Lohse et al. 2014). Processed reads were mapped to the mm10 reference genome using HISAT2 (2.0.4) (Kim, Langmead et al. 2015). Counts were then assembled using Subread featureCounts (1.5.2) in a custom bash script (Liao, Smyth et al. 2014). Differential gene expression analysis was done using the DESeq2 package in R72. Gene ontology analysis was performed using the PANTHER Version 12.0 classification 73, 74. Canonical pathway analysis was done using Ingenuity Pathway Analysis (QIAGEN Inc.). To perform surface receptor analysis, list of candidate surface receptors was identified from the UniProtKB/Swiss-Prot database using the search terms “Gene Ontology: transmembrane signaling receptor activity” and “Organism: Mus musculus.”

RNA-Sequencing Analysis

Raw sequencing reads were trimmed using Trimmomatic (0.36) with a minimum quality threshold of 35 and minimum length of 36⁷⁵. Processed reads were mapped to the mm10 reference genome using HISAT2 (2.0.4)⁷⁶. Counts were then assembled using Subread featureCounts (1.5.2) in a custom bash script⁷⁷.

Statistical Analyses

All studies were done with at least three sets of independent experiments. Two-group analysis used Student's t test. Comparisons of multiple groups were performed using either one-way or two-way ANOVA. P value<0.05 was considered significant. For RNA-seq analysis, Benjamini-Hochberg correction was used to adjust for multiple testing, with threshold of adjusted p-value<0.1 (i.e. false discovery rate<10%) considered significant.

### REFERENCES

The following references are identified above often by superscript number that designates the corresponding numbered documents as set forth below.
1. Arkell, R. M., Fossat, N. & Tam, P. P. Wnt signalling in mouse gastrulation and anterior development: new players in the pathway and signal output. Curr Opin Genet Dev 23, 454-460 (2013).
2. Cornell, R. A. & Kimelman, D. Activin-mediated mesoderm induction requires FGF. Development 120, 453-462 (1994).
3. Weinstein, D. C., Marden, J., Carnevali, F. & Hemmati-Brivanlou, A. FGF-mediated mesoderm induction involves the Src-family kinase Laloo. Nature 668 394, 904-908 (1998).
4. Winnier, G., Blessing, M., Labosky, P. A. & Hogan, B. L. Bone morphogenetic protein-4 is required for mesoderm formation and patterning in the mouse. Genes Dev 9, 2105-2116 (1995).
5. Cheng, P. et al. Fibronectin mediates mesendodermal cell fate decisions. Development 140, 2587-2596 (2013).
6. Galdos, F. X. et al. Cardiac Regeneration: Lessons From Development. Circ Res 120, 941-959 (2017).
7. Devine, W. P., Wythe, J. D., George, M., Koshiba-Takeuchi, K. & Bruneau, B. G. Early patterning and specification of cardiac progenitors in gastrulating mesoderm. Elife 3 (2014).
8. Lescroart, F. et al. Early lineage restriction in temporally distinct populations of Mesp1 progenitors during mammalian heart development. Nat Cell Biol 16, 681 829-840 (2014).
9. Bruneau, B. G. Signaling and transcriptional networks in heart development and regeneration. Cold Spring Harb Perspect Biol 5, a008292 (2013).
10. Kelly, R. G., Buckingham, M. E. & Moorman, A. F. Heart fields and cardiac morphogenesis. Cold Spring Harb Perspect Med 4 (2014).
11. Bondue, A. et al. Mesp1 acts as a master regulator of multipotent cardiovascular progenitor specification. Cell Stem Cell 3, 69-84 (2008).
12. Costello, I. et al. The T-box transcription factor Eomesodermin acts upstream of Mesp1 to specify cardiac mesoderm during mouse gastrulation. Nat Cell Biol 13, 1084-1091 (2011).
13. Cunningham, T. J. et al. Id genes are essential for early heart formation. GenesDev (2017).
14. Saga, Y. et al. MesP1 is expressed in the heart precursor cells and required for the formation of a single heart tube. Development 126, 3437-3447 (1999).
15. Spater, D. et al. A HCN4+ cardiomyogenic progenitor derived from the first heart field and human pluripotent stem cells. Nat Cell Biol 15, 1098-1106 (2013).
16. Bruneau, B. G. et al. Chamber-specific cardiac expression of Tbx5 and heart defects in Holt-Oram syndrome. Dev Biol 211, 100-108 (1999).
17. Huynh, T., Chen, L., Terrell, P. & Baldini, A. A fate map of Tbx1 expressing cells reveals heterogeneity in the second cardiac field. Genesis 45, 470-475 (2007).
18. Watanabe, Y. et al. Fibroblast growth factor 10 gene regulation in the second heart field by Tbx1, Nkx2-5, and Islet1 reveals a genetic switch for down-regulation in the myocardium. Proc Natl Acad Sci USA 109, 18273-18280 (2012).
19. Zhou, Z. et al. Temporally Distinct Six2-Positive Second Heart Field Progenitors Regulate Mammalian Heart Development and Disease. Cell Rep 18, 1019-1032 (2017).
20. Francou, A. et al. Second heart field cardiac progenitor cells in the early mouse embryo. Biochim Biophys Acta 1833, 795-798 (2013).
21. Cai, C. L. et al. Isl1 identifies a cardiac progenitor population that proliferates prior to differentiation and contributes a majority of cells to the heart. Dev Cell 5, 877-889 (2003).
22. Meilhac, S. M., Esner, M., Kelly, R. G., Nicolas, J. F. & Buckingham, M. E. The clonal origin of myocardial cells in different regions of the embryonic mouse heart. Dev Cell 6, 685-698 (2004).
23. Brade, T., Pane, L. S., Moretti, A., Chien, K. R. & Laugwitz, K. L. Embryonic heart progenitors and cardiogenesis. Cold Spring Harb Perspect Med 3, a013847 (2013).
24. Fox, I. J. et al. Stem cell therapy. Use of differentiated pluripotent stem cells as replacement therapy for treating disease. Science 345, 1247391 (2014).
25. Grskovic, M., Javaherian, A., Strulovici, B. & Daley, G. Q. Induced pluripotent stem cells—opportunities for disease modelling and drug discovery. Nat Rev Drug Discov 10, 915-929 (2011).
26. Cho, G. S. et al. Neonatal Transplantation Confers Maturation of PSC-Derived Cardiomyocytes Conducive to Modeling Cardiomyopathy. Cell Rep 18, 571-727 582 (2017).
27. Kattman, S. J. et al. Stage-specific optimization of activin/nodal and BMP signaling promotes cardiac differentiation of mouse and human pluripotent stem cell lines. Cell Stem Cell 8, 228-240 (2011).
28. Takahashi, K. et al. Induction of pluripotent stem cells from adult human fibroblasts by defined factors. Cell 131, 861-872 (2007).
29. Liu, X. et al. The complex genetics of hypoplastic left heart syndrome. Nat Genet 49, 1152-1159 (2017).
30. Li, L. et al. HAND1 loss-of-function mutation contributes to congenital double outlet right ventricle. Int J Mol Med (2017).
31. Corrado, D., Link, M. S. & Calkins, H. Arrhythmogenic Right Ventricular Cardiomyopathy. N Engl J Med 376, 61-72 (2017).
32. Maron, B. J. & Maron, M. S. Hypertrophic cardiomyopathy. Lancet 381, 242-255 (2013).
33. Morita, Y. et al. Sall1 transiently marks undifferentiated heart precursors and regulates their fate. J Mol Cell Cardiol 92, 158-162 (2016).
34. Garg, V. et al. Mutations in NOTCH1 cause aortic valve disease. Nature 437, 270-274 (2005).
35. Nguyen, M. D. et al. Cardiac cell culture model as a left ventricle mimic for cardiac tissue generation. Anal Chem 85, 8773-8779 (2013).
36. Ong, C. S. et al. Biomaterial-Free Three-Dimensional Bioprinting of Cardiac Tissue using Human Induced Pluripotent Stem Cell Derived Cardiomyocytes. Sci Rep 7, 4566 (2017).
37. Park, E. J. et al. Required, tissue-specific roles for Fgf8 in outflow tract formation and remodeling. Development 133, 2419-2433 (2006).
38. Vedantham, V., Evangelista, M., Huang, Y. & Srivastava, D. Spatiotemporal regulation of an Hcn4 enhancer defines a role for Mef2c and HDACs in cardiac electrical patterning. Dev Biol 373, 149-162 (2013).
39. Mjaatvedt, C. H. et al. The outflow tract of the heart is recruited from a novel heart-forming field. Dev Biol 238, 97-109 (2001).
40. Prall, O. W. et al. An Nkx2-5/Bmp2/Smad1 negative feedback loop controls heart progenitor specification and proliferation. Cell 128, 947-959 (2007).
41. Kokkinopoulos, I. et al. Single-Cell Expression Profiling Reveals a Dynamic State of Cardiac Precursor Cells in the Early Mouse Embryo. PLoS One 10, e0140831 (2015).
42. Srinivas, S. et al. Cre reporter strains produced by targeted insertion of EYFP and ECFP into the ROSA26 locus. BMC Dev Biol 1, 4 (2001).
43. Shenje, L. T. et al. Precardiac deletion of Numb and Numblike reveals renewal of cardiac progenitors. Elife 3, e02164 (2014).
44. Dastjerdi, A. et al. Tbx1 regulation of myogenic differentiation in the limb and cranial mesoderm. Dev Dyn 236, 353-363 (2007).
45. Bruneau, B. G. et al. A murine model of Holt-Oram syndrome defines roles of the T-box transcription factor Tbx5 in cardiogenesis and disease. Cell 106, 709-721 (2001).
46. Koshiba-Takeuchi, K. et al. Reptilian heart development and the molecular basis of cardiac chamber evolution. Nature 461, 95-98 (2009).
47. Kramer, A., Green, J., Pollard, J., Jr. & Tugendreich, S. Causal analysis approaches in Ingenuity Pathway Analysis. Bioinformatics 30, 523-530(2014).
48. Uosaki, H. et al. Transcriptional Landscape of Cardiomyocyte Maturation. Cell Rep 13, 1705-1716 (2015).
49. Zimmerman, L. B., De Jesus-Escobar, J. M. & Harland, R. M. The Spemann organizer signal noggin binds and inactivates bone morphogenetic protein 4. Cell 86, 599-606 (1996).
50. Hao, J. et al. DMH1, a small molecule inhibitor of BMP type i receptors, suppresses growth and invasion of lung cancer. PLoS One 9, e90748 (2014).
51. Yu, P. B. et al. Dorsomorphin inhibits BMP signals required for embryogenesis and iron metabolism. Nat Chem Biol 4, 33-41 (2008).
52. Cho, G. S., Fernandez, L. & Kwon, C. Regenerative medicine for the heart: perspectives on stem-cell therapy. Antioxid Redox Signal 21, 2018-2031 (2014).
53. Kelly, R. G., Brown, N. A. & Buckingham, M. E. The arterial pole of the mouse heart forms from Fgf10-expressing cells in pharyngeal mesoderm. Dev Cell 1, 435-440 (2001).
54. Ieda, M. et al. Direct reprogramming of fibroblasts into functional cardiomyocytes by defined factors. Cell 142, 375-386 (2010).
55. Cho, G. S., Tampakakis, E., Andersen, P. & Kwon, C. Use of a neonatal rat system as a bioincubator to generate adult-like mature cardiomyocytes from human and mouse pluripotent stem cells. Nat Protoc 12, 2097-2109 (2017).
56. Domian, I. J. et al. Generation of functional ventricular heart muscle from mouse ventricular progenitor cells. Science 326, 426-429 (2009).
57. Ma, Q., Zhou, B. & Pu, W. T. Reassessment of Isl1 and Nkx2-5 cardiac fate maps using a Gata4-based reporter of Cre activity. Dev Biol 323, 98-104 (2008).
58. Laugwitz, K. L., Moretti, A., Caron, L., Nakano, A. & Chien, K. R. Islet1 cardiovascular progenitors: a single source for heart lineages? Development 135, 193-205 (2008).
59. Hollnagel, A., Oehlmann, V., Heymer, J., Ruther, U. & Nordheim, A. Id genes are direct targets of bone morphogenetic protein induction in embryonic stem cells. J Biol Chem 274, 9838-19845 (1999).
60. Katagiri, T. et al. Identification of a BMP-responsive element in Id1, the gene for inhibition of myogenesis. Genes Cells 7, 949-960 (2002).
61. Korchynskyi, O. & ten Dijke, P. Identification and functional characterization of distinct critically important bone morphogenetic protein-specific response elements in the Id1 promoter. J Biol Chem 277, 4883-4891 (2002).
62. Lee, J. H., Protze, S. I., Laksman, Z., Backx, P. H. & Keller, G. M. Human Pluripotent Stem Cell-Derived Atrial and Ventricular Cardiomyocytes Develop from Distinct Mesoderm Populations. Cell Stem Cell 21, 179-194 e174 (2017).
63. Klaus, A., Saga, Y., Taketo, M. M., Tzahor, E. & Birchmeier, W. Distinct roles of Wnt/beta-catenin and Bmp signaling during early cardiogenesis. Proc Natl Acad Sci USA 104, 18531-18536 (2007).
64. Kwon, C., Cordes, K. R. & Srivastava, D. Wnt/beta-catenin signaling acts at multiple developmental stages to promote mammalian cardiogenesis. Cell Cycle 7, 3815-3818 (2008).
65. Camp, J. G. et al. Human cerebral organoids recapitulate gene expression programs of fetal neocortex development. Proc Natl Acad Sci USA 112, 15672-15677 (2015).
66. Van der Hauwaert, L. G. & Michaelsson, M. Isolated right ventricular hypoplasia. Circulation 44, 466-474 (1971).
67. Harris, K. C. et al. Right ventricular outflow tract tachycardia in children. J Pediatr 149, 822-826 (2006).
68. Uosaki, H. et al. Direct contact with endoderm-like cells efficiently induces cardiac progenitors from mouse and human pluripotent stem cells. PLoS One 7, e46413 (2012).
69. Kwon, C. et al. A regulatory pathway involving Notch1/beta-catenin/Isl1 determines cardiac progenitor cell fate. Nat Cell Biol 11, 951-957 (2009).
70. Shin, J. et al. Single-Cell RNA-Seq with Waterfall Reveals Molecular Cascades underlying Adult Neurogenesis. Cell Stem Cell 17, 360-372 (2015).
71. Picelli, S. et al. Tn5 transposase and tagmentation procedures for massively scaled sequencing projects. Genome Res 24, 2033-2040 (2014).
72. Anders, S., Pyl, P. T. & Huber, W. HTSeq—a Python framework to work with high-throughput sequencing data. Bioinformatics 31, 166-169 (2015).
73. Mi, H. et al. PANTHER version 11: expanded annotation data from Gene Ontology and Reactome pathways, and data analysis tool enhancements. Nucleic Acids Res 45, D183-D189 (2017).
74. Mi, H., Muruganujan, A., Casagrande, J. T. & Thomas, P. D. Large-scale gene function analysis with the PANTHER classification system. Nat Protoc 8, 1551-1566 (2013).
75. Bolger, A. M., Lohse, M. & Usadel, B. Trimmomatic: a flexible trimmer for Illumina sequence data. Bioinformatics 30, 2114-2120 (2014).
76. Kim, D., Langmead, B. & Salzberg, S. L. HISAT: a fast spliced aligner with low memory requirements. Nat Methods 12, 357-360 (2015).
77. Liao, Y., Smyth, G. K. & Shi, W. featureCounts: an efficient general purpose program for assigning sequence reads to genomic features. Bioinformatics 30, 923-930 (2014).

### EQUIVALENTS

Those skilled in the art will recognize, or be able to ascertain using no more than routine experimentation, many equivalents to the specific embodiments of the invention described herein.

Such equivalents are intended to be encompassed by the following claims.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All united states patents and published or unpublished united states patent applications cited herein are incorporated by reference. All published foreign patents and patent applications cited herein are hereby incorporated by reference. Genbank and NCBI submissions indicated by accession number cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method of producing first heart field (FHF) induced pluripotent stem (iPS) cell or second heart field (SHF) iPS, the method comprising:
a) providing a population of pluripotent stem cells from a mammal, wherein a cluster of cells is formed,
b) contacting the cluster of cells with one or more reprogramming factors, and thereby producing FHF iPS cells or SHF iPS cells.

2. The method of claim 1, wherein the one or more reprogramming factors comprises a transforming growth factor beta (TGF-0) protein, a Wnt protein, or a bone morphogenic protein (Bmp).

3. The method of claim 2, wherein the Wnt protein comprises Wnt3A, Wnt5A or Wnt11.

4. The method of claim 2, wherein the Bmp protein comprises Bmp4.

5. The method of claim 2, wherein the TGF-β protein comprises Activin A.

6. The method of claim 1, further comprising providing a Wnt pathway activator.

7. The method of claim 6, wherein the Wnt pathway activator comprises a glycogen synthesis kinase 3 (Gsk3) inhibitor.

8. The method of claim 1, wherein the iPSC is genetically modified to alter the expression or activity of C-X-C chemokine receptor type 4 (Cxcr4), and thereby producing SHF iPS cells.

9. The method of claim 1, wherein the iPS cells are human iPS (hiPS) cells.

10. The method of claim 1, wherein the mammal is selected from a group consisting of: rodents, rats, mice, rabbits, goats, non-human primates, humans, dogs, bears, cats, lions, tigers, elephants, llamas, donkeys, mules, bovines, ovines, pigs, and horses.

11. A method of treating a disease or condition comprising administering to a subject, the iPS cells produced by a method of claim 1.

12. The method of claim 11, wherein the iPS cells are administered via oral administration, intravenous administration, topical administration, parenteral administration, intraperitoneal administration, intramuscular administration, intrathecal administration, intralesional administration, intracranial administration, intranasal administration, intraocular administration, intracardiac administration, intravitreal administration, intraosseous administration, intracerebral administration, intraarterial administration, intraarticular administration, intradermal administration, transdermal administration, transmucosal administration, sublingual administration, enteral administration, sublabial administration, insufflation administration, suppository administration, inhaled administration, intraventricular injection, or subcutaneous administration

13. A cell comprising an agent that alters the expression or activity of C-X-C chemokine receptor type 4 (Cxcr4).

14. The cell of claim 13, wherein the cell comprises a genetically modified stem cell, mesenchymal stem cell, induced pluripotent stem cell (iPSC), iPSC-derived pericytes, or iPSC-derived cardiac muscle cell.

15. The cell of claim 13, wherein the agent is selected from the group consisting of an antibody or fragment thereof, a peptide, a polypeptide or fragments thereof, a small molecule, and a nucleic acid.

16. A method for treating or preventing a heart disease in a subject, comprising administering a genetically modified stem cell, mesenchymal stem cell, induced pluripotent stem cell (iPSC), iPSC-derived pericytes, or iPSC-derived cardiac muscle cell to the subject.

17. The method of claim 16, wherein the stem cell, the mesenchymal stem cell or the iPSC is derived from the subject.

18. The method of claim 16, wherein the stem cell or the iPSC has been genetically modified to alter the expression or activity of C-X-C chemokine receptor type 4 (Cxcr4).

19. The method of claim 18, wherein the expression or activity of Cxcr4 is altered by an agent, wherein the agent is selected from the group consisting of an antibody or fragment thereof, a peptide, a polypeptide or fragments thereof, a small molecule, and a nucleic acid.

19. (canceled)

20. A composition comprising induced pluripotent stem cells comprising a vector encoding C-X-C chemokine receptor type 4 (Cxcr4).
